SEQUENCE LISTING (113 sequences)

SEQ ID NO: 1 - DNA, 193 bp, Pseudomonas aeruginosa
tacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaata

SEQ ID NO: 2 - DNA, 192 bp, Pseudomonas aeruginosa
tacgggaggcagcagtggggaatattggacaagggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaata

SEQ ID NO: 3 - DNA, 193 bp, Pseudomonas aeruginosa; identical to SEQ ID NO: 1

SEQ ID NO: 4 - DNA, 193 bp, Pseudomonas aeruginosa; identical to SEQ ID NO: 1

SEQ ID NO: 5 - DNA, 193 bp, Pseudomonas aeruginosa; identical to SEQ ID NO: 1

SEQ ID NO: 6 - DNA, 193 bp, Pseudomonas putida
tacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaactctgtgccagcagccgcggtaata

SEQ ID NO: 7 - DNA, 193 bp, Enterobacter aerogenes
tacgggaggcagcagtggggaatattgcacaatgggcgcaagcctgatgcagccatgccgcgtgtatgaagaaggccttcgggttgtaaagtactttcagcgaggaggaaggcgttaaggttaataaccttggcgattgacgttactcgcagaagaagcaccggctaactccgtgccagcagccgcggtaata

SEQ ID NO: 8 - DNA, 193 bp, Enterobacter sakazakii
tacgggaggcagcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaata

SEQ ID NO: 9 - DNA, 193 bp, Enterobacter cloacae
tacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaata

SEQ ID NO: 10 - DNA, 193 bp, Citrobacter koseri; identical to SEQ ID NO: 9

SEQ ID NO: 11 - DNA, 193 bp, Citrobacter freundii
tacgggaggcagcagtggggaatattgcacaatgggcacaagactgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaata

SEQ ID NO: 12 - DNA, 193 bp, Citrobacter freundii
tacgggaggcagcagtggggaatattgcacaatgggcgcaagcctgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaata

SEQ ID NO: 13 - DNA, 193 bp, Klebsiella terrigena
tacgggaggcagcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaata

SEQ ID NO: 14 - DNA, 193 bp, Klebsiella pneumoniae
tacgggaggcagcagtggggaatattgcacaatgggcgcaagcctgatgcagccatgccgcgtgtgtgaagaaggccttcgggttgtaaagcactttcagcggggaggaaggcggtgaggttaataacctcaccgattgacgttacccgcagaagaagcaccggctaactccgtgccagcagccgcggtaata

SEQ ID NO: 15 - DNA, 193 bp, Citrobacter youngae
tacgggaggcagcagtggggaatattgcacaatgggcgcaagcctgatgcagccatgccgcgtgtgtgaagaaggccttcgggttgtaaagcactttcagcggggaggaaggcggtaaggttaataacctcgtcgattgacgttacccgcagaagaagcaccggctaactccgtgccagcagccgcggtaata

SEQ ID NO: 16 - DNA, 193 bp, Klebsiella pneumoniae
tacgggaggcagcagtggggaatattgcacaatgggcgcaagcctgatgcagccatgccgcgtgtgtgaagaaggccttcgggttgtaaagcactttcagcggggaggaaggcgttgaggttaataacctcggcgattgacgttacccgcagaagaagcaccggctaactccgtgccagcagccgcggtaata

SEQ ID NO: 17 - DNA, 193 bp, Proteus vulgaris
tacgggaggcagcagtggggaatattgcacaatgggcgcaagcctgatgcagccatgccgcgtgtatgaagaaggccctagggttgtaaagtactttcagtcgggaggaaggcgttgatgttaataccatcaacgattgacgttaccgacagaagaagcaccggctaactccgtgccagcagccgcggtaata

SEQ ID NO: 18 - DNA, 193 bp, Providencia stuartii; identical to SEQ ID NO: 17

SEQ ID NO: 19 - DNA, 193 bp, Morganella morganii
tacgggaggcagcagtggggaatattgcacaatgggcgcaagcctgatgcagccatgccgcgtgtatgaagaaggccttcgggttgtaaagtactttcagtcgggaggaaggtgtcaaggttaataaccttggcaattgacgttaccgacagaagaagcaccggctaactccgtgccagcagccgcggtaata

SEQ ID NO: 20 - DNA, 193 bp, Escherichia coli
tacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaata

SEQ ID NO: 21 - DNA, 193 bp, Escherichia coli; identical to SEQ ID NO: 20

SEQ ID NO: 22 - DNA, 193 bp, Shigella flexneri; identical to SEQ ID NO: 20

SEQ ID NO: 23 - DNA, 193 bp, Klebsiella oxytoca
tacgggaggcagcagtggggaatattgcacaatgggcgcaagcctgatgcagccatgccgcgtgtatgaagaaggccttcgggttgtaaagtactttcagcggggaggaagggagtgaggttaataaccttattcattgacgttacccgcagaagaagcaccggctaactccgtgccagcagccgcggtaata

SEQ ID NO: 24 - DNA, 193 bp, Proteus mirabilis
tacgggaggcagcagtggggaatattgcacaatgggcgcaagcctgatgcagccatgccgcgtgtatgaagaaggccttagggttgtaaagtactttcagcggggaggaaggtgataaggttaatacccttatcaattgacgttacccgcagaagaagcaccggctaactccgtgccagcagccgcggtaata

SEQ ID NO: 25 - DNA, 193 bp, Proteus mirabilis; identical to SEQ ID NO: 24

SEQ ID NO: 26 - DNA, 193 bp, Serratia marcescens
tacgggaggcagcagtggggaatattgcacaatgggcgcaagcctgatgcagccatgccgcgtgtgtgaagaaggccttcgggttgtaaagcactttcagcgaggaggaaggtggtgaacttaatacgttcatcaattgacgttactcgcagaagaagcaccggctaactccgtgccagcagccgcggtaata

SEQ ID NO: 27 - DNA, 194 bp, Acinetobacter baumannii
tacgggaggcagcagtggggaatattggacaatggggggaaccctgatccagccatgccgcgtgtgtgaagaaggccttatggttgtaaagcactttaagcgaggaggaggctactttagttaatacctagagatagtggacgttactcgcagaataagcaccggctaactctgtgccagcagccgcggtaata

SEQ ID NO: 28 - DNA, 193 bp, Enterococcus faecalis
tacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaata

SEQ ID NO: 29 - DNA, 193 bp, Enterococcus faecalis; identical to SEQ ID NO: 28

SEQ ID NO: 30 - DNA, 193 bp, Enterococcus faecium
tacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaata

SEQ ID NO: 31 - DNA, 194 bp, Streptococcus pyogenes
tacgggaggcagcagtagggaatcttcggcaatgggggcaaccctgaccgagcaacgccgcgtgagtgaagaaggttttcggatcgtaaagctctgttgttagagaagaatgatggtgggagtggaaaatccaccaagtgacggtaactaaccagaaagggacggctaactacgtgccagcagccgcggtaata

SEQ ID NO: 32 - DNA, 194 bp, Streptococcus agalactiae
tacgggaggcagcagtagggaatcttcggcaatggacggaagtctgaccgagcaacgccgcgtgagtgaagaaggttttcggatcgtaaagctctgttgttagagaagaacgttggtaggagtggaaaatctaccaagtgacggtaactaaccagaaagggacggctaactacgtgccagcagccgcggtaata

SEQ ID NO: 33 - DNA, 194 bp, Streptococcus pneumoniae
tacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaata

SEQ ID NO: 34 - DNA, 193 bp, Staphylococcus aureus
tacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaata

SEQ ID NO: 35 - DNA, 193 bp, Staphylococcus aureus; identical to SEQ ID NO: 34

SEQ ID NO: 36 - DNA, 193 bp, Staphylococcus epidermidis
tacgggaggcagcagtagggaatctaccacaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaata

SEQ ID NO: 37 - DNA, 193 bp, Staphylococcus epidermidis
tacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaata

SEQ ID NO: 38 - DNA, 193 bp, Staphylococcus epidermidis; identical to SEQ ID NO: 37

SEQ ID NO: 39 - DNA, 193 bp, Pseudomonas fluorescens
tacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaata

SEQ ID NO: 40 - DNA, 16 bp, Pseudomonas aeruginosa
tacgggaggcagcagt

SEQ ID NO: 41 - DNA, 16 bp, Pseudomonas aeruginosa
agcagccgcggtaata

SEQ ID NO: 42 - DNA, 16 bp, Enterococcus faecalis
gagcaacgccgcgtga

SEQ ID NO: 43 - DNA, 16 bp, Pseudomonas fluorescens
cagccatgccgcgtgt

SEQ ID NO: 44 - DNA, 23 bp, Staphylococcus aureus
gatggtatgtggaagttagattg

SEQ ID NO: 45 - DNA, 22 bp, Staphylococcus aureus
gaccgaaacaatgtggaattgg

SEQ ID NO: 46 - DNA, 22 bp, Staphylococcus aureus
gatggctatcgtgtcacaatcg

SEQ ID NO: 47 - DNA, 20 bp, Staphylococcus aureus
ttcatatgacgtctatccat

SEQ ID NO: 48 - DNA, 19 bp, Staphylococcus aureus
catagcgtcattattccag

SEQ ID NO: 49 - DNA, 29 bp, Staphylococcus aureus
catagcgtcattattccaggaatgcagaa

SEQ ID NO: 50 - DNA, 24 bp, Staphylococcus aureus
gattatggctcaggtactgctatc

SEQ ID NO: 51 - DNA, 20 bp, Staphylococcus aureus
agagtttgatchtggctcag

SEQ ID NO: 52 - DNA, 20 bp, Staphylococcus aureus
cctactgctgcctccctgta

SEQ ID NO: 53 - DNA, 15 bp, Staphylococcus aureus
ttctctgatgttagc

SEQ ID NO: 54 - DNA, 20 bp, Staphylococcus epidermidis
agagtttgatchtggctcag

SEQ ID NO: 55 - DNA, 20 bp, Staphylococcus epidermidis
cctactgctgcctccctgta

SEQ ID NO: 56 - DNA, 15 bp, Staphylococcus epidermidis
tcctctgacgttagc

SEQ ID NO: 57 - DNA, 169 bp, Enterobacter cloacae
gggatgacgtcaagtcatcatggcccttacgaccagggctacacacgagaaacaatggcgcatacaaagagaagcgacctcgcgagagcaagcggacctcataaagtgcgtcgtagtccggattggagtctgcaactcgactccatgaagtcggaatcgctagtaatcg

SEQ ID NO: 58 - DNA, 183 bp, Pseudomonas aeruginosa
gaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtatccgta

SEQ ID NO: 59 - DNA, 183 bp, Acinetobacter baumannii
gaagtcggaatcgctagtaatcgcggatcagaatgccgcggtgaatacgttcccgggccttgtacacaccgcccgtcacaccatgggagtttgttgcaccagaagtagctagcctaactgcaaagagggcggttaccacggtgtggccgatgactggggtgaagtcgtaacaaggtatccgta

SEQ ID NO: 60 - DNA, 353 bp, Citrobacter koseri
gagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagttggtggggtaacggctcaccaaggcgacgatccctagctggtctgagaggatgaccgccacactggaactgagacacggtccagactcctacgggaggcagcagtgggg

SEQ ID NO: 61 - DNA, 352 bp, Klebsiella pneumoniae
gagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataatgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtgggg

SEQ ID NO: 62 - DNA, 168 bp, Proteus mirabilis
ggaagacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcg

SEQ ID NO: 63 - DNA, 354 bp, Morganella moranii
gagtttgatcatggctcagattgatcgctggcggcaggcctaacacatgcaagtcgggcggtaacagggagaagcttgcttctctgctgacgagcggcggacgggtgagtaatgtatggggatctgcctgatggcgggggttaactactggaaacggtagctaataccgcataatgtctacggaccaaagcgggggacctccgggcctcgcgccatcagatgaacccatatgggattagctagtaggtaaggtaacggcttacctaggcgacgatccctagctggtctgagaggatgatcagccacactgggactgagacacggcccagactcctacgggaggcagcagtgggg

SEQ ID NO: 64 - DNA, 168 bp, Enterococcus faecalis
ggatgaagacaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcg

SEQ ID NO: 65 - DNA, 169 bp, Enterococcus faecium
gggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcg

SEQ ID NO: 66 - DNA, 183 bp, Streptococcus pneumoniae
gaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtatccgta

SEQ ID NO: 67 - DNA, 184 bp, Streptococcus pyogenes
gaagtcggaatcgctagtaatcgcggatcagcacgccgcggtgaatacgttcccgggccttgtacacaccgcccgtcacaccacgagagtttgtaacacccgaagtcggtgaggtaacctattaggagccagccgcctaaggtgggatagatgattggggtgaagtcgtaacaaggtatccgta

SEQ ID NO: 68 - DNA, 361 bp, Staphylococcus aureus
gagtttgatcatggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtaggg

SEQ ID NO: 69 - DNA, 362 bp, Staphylococcus epidermidis
gagtttgatcatggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggg

SEQ ID NO: 70 - DNA, 348 bp, Pseudomonas putida
gagtttgatcttggctcagattgaacgctggcggcaggcctaacacatgcaagtcgagcggatgacgggagcttgctccttgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtgggg

SEQ ID NO: 71 - DNA, 354 bp, Citrobacter freundii
gagtttgatcttggctcagattgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtgggg

SEQ ID NO: 72 - DNA, 355 bp, Enterobacter sakazakii
gagtttgatcttggctcagattgaacgctggcggcaggcctaacacatgcaagtcgaacggtagcacagaggaggcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtgggg

SEQ ID NO: 73 - DNA, 354 bp, Klebsiella terrigena
gagtttgatcttggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtgggg

SEQ ID NO: 74 - DNA, 352 bp, Klebsiella oxytoca
gagtttgatcttggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataatgtcgcaagaccaaagagggggaccttcgggcctcttgccatcggatgtgcccagatgggattagcttgtaggtgaggtaacggctcacctaggcgacgatccctagctggtctgagaggatgaccagccacactggaactgagacacggtccagactcctacgggaggcagcagtgggg

SEQ ID NO: 75 - DNA, 354 bp, Serratia marcescens
gagtttgatcttggctcagattgaacgctggcggcaggcttaacacatgcaagtcgagcggtagcacaagggagcttgctccctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtgggg

SEQ ID NO: 76 - DNA, 361 bp, Staphylococcus lugdunensis
gagtttgatcttggctcaggatgaacgctggcggcgtgcctaatacatgcaagtcgagcgaacagataaggagcttgctcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtaggg

SEQ ID NO: 77 - DNA, 352 bp, Providecia stuartii
gagtttgatcttggctcagattgaacgctggcggcaggcctaacacatgcaagtcgagcggtaacaggggaagcttgcttctcgctgacgagcggcggacgggtgagtaatgtatggggatctgcccgatagagggggataactactggaaacggtggctaataccgcataatctcttaggagcaaagcaggggaccttcgggccttgcgctgtcggatgaacccatatgggattagctagtaggtaaggtaatggcttacctaggcgacgatccctagctggtctgagaggatgatcagccacactgggactgagacacggcccagactcctacgggaggcagcagtgggg

SEQ ID NO: 78 - DNA, 360 bp, Streptococcus agalactiae
gagtttgatcttggctcaggacgaacgctggcggcgtgcctaatacatgcaagtagaacgctgaggtttggtgtttacactagactgatgagttgcgaacgggtgagtaacgcgtaggtaacctgcctcatagcgggggataactattggaaacgatagctaataccgcataagagtaattaacacatgttagttatttaaaaggagcaattgcttcactgtgagatggacctgcgttgtattagctagttggtgaggtaaaggctcaccaaggcgacgatacatagccgacctgagagggtgatcggccacactgggactgagacacggcccagactcctacgggaggcagcagtaggg

SEQ ID NO: 79 - DNA, 132 bp, Staphylococcus aureus
gataagcattggaaattagattggagaccagacgtaatagtacctggtttgaaaaatggacagaaaattaatatagaaacattaaaatcagagcgaggcaaaataaaagatagaaatggtatagaattagct

SEQ ID NO: 80 - DNA, 23 bp, Staphylococcus aureus
gataagcattggaaattagattg

SEQ ID NO: 81 - DNA, 24 bp, Staphylococcus aureus
agctaattctatattgtttcggtc

SEQ ID NO: 82 - DNA, 19 bp, Staphylococcus aureus
ccagacgtaatagtacctg

SEQ ID NO: 83 - DNA, 20 bp, Staphylococcus aureus
agagtttgatchtggctcag

SEQ ID NO: 84 - DNA, 19 bp, Staphylococcus aureus
ccyactgctgcctcccgta

SEQ ID NO: 85 - DNA, 21 bp, Artificial Sequence
  synthetic nucleic acid
gggahgamgwcaartcatcat

SEQ ID NO: 86 - DNA, 23 bp, Artificial Sequence
  synthetic nucleic acid
cgattactagcgattccrrcttc

SEQ ID NO: 87 - DNA, 23 bp, Artificial Sequence
  synthetic nucleic acid
gaagyyggaatcgctagtaatcg

SEQ ID NO: 88 - DNA, 21 bp, Artificial Sequence
  synthetic nucleic acid
tacrghtaccttgttacgact

SEQ ID NO: 89 - DNA, 15 bp, Enterobacter cloacae
gaaaacaatggcgca

SEQ ID NO: 90 - DNA, 15 bp, Pseudomonas aeruginosa
acggttaccacggag

SEQ ID NO: 91 - DNA, 18 bp, Acinetobacter baumannii
ctagcctaactgcaaaga

SEQ ID NO: 92 - DNA, 15 bp, Citrobacter koseri
gatgaccgccacact

SEQ ID NO: 93 - DNA, 14 bp, Citrobacter koseri
gcctcttgccatca

SEQ ID NO: 94 - DNA, 14 bp, Klebsiella pneumonia
gcctcatgccatca

SEQ ID NO: 95 - DNA, 15 bp, Proteus mirabilis
ggcagatacaaagag

SEQ ID NO: 96 - DNA, 15 bp, Morganella moranii
ggcgtatacaaaggg

SEQ ID NO: 97 - DNA, 15 bp, Morganella moranii
gatctgcctgatggc

SEQ ID NO: 98 - DNA, 15 bp, Enterococcus faecalis
aacgagtcgctagac

SEQ ID NO: 99 - DNA, 15 bp, Enterococcus faecium
aacgagttgcgaagt

SEQ ID NO: 100 - DNA, 15 bp, Streptococcus pneumonia
accgtaaggagccag

SEQ ID NO: 101 - DNA, 15 bp, Streptococcus pyogenes
cctattaggagccag

SEQ ID NO: 102 - DNA, 15 bp, Staphylococcus aureus
ttctctgatgttagc

SEQ ID NO: 103 - DNA, 15 bp, Staphylococcus epidermidis
tcctctgacgttagc

SEQ ID NO: 104 - DNA, 16 bp, Pseudomonas putida
caacgtttccaaagga

SEQ ID NO: 105 - DNA, 16 bp, Citrobacter freundii
tagcacagaggagctt

SEQ ID NO: 106 - DNA, 17 bp, Enterobacter sakazakii
tagcacagaggaggctt

SEQ ID NO: 107 - DNA, 17 bp, Klebsiella terrigena
cagcttgctgcttcgct

SEQ ID NO: 108 - DNA, 16 bp, Klebsiella oxytoca
ttgtaggtgaggtaac

SEQ ID NO: 109 - DNA, 17 bp, Serratia marcescens
tagcacaagggagcttg

SEQ ID NO: 110 - DNA, 16 bp, Staphylococcus lugdunensis
ggacccgcgccgtatt

SEQ ID NO: 111 - DNA, 18 bp, Providecia stuartii
atctcttaggagcaaagc

SEQ ID NO: 112 - DNA, 17 bp, Streptococcus agalactiae
ctgaggtttggtgttta

SEQ ID NO: 113 - DNA, 168 bp, Morganella moranii
ggatgacgtcaagtcatcatggcccttacgagtagggctacacacgtgctacaatggcgtatacaaagggaagcgaccccgcgagggcaagcggaactcataaagtacgtcgtagtccggattggagtctgcaactcgactccatgaagtcggaatcgctagtaatcg

(19) United States
(12) Patent Application Publication
(10) Pub. No.: US 2014/0378337 A1
(43) Pub. Date: Dec. 25, 2014

(54) COMPOSITIONS AND METHODS FOR DETECTING AND IDENTIFYING BACTERIA

(71) Applicant: WAKE FOREST UNIVERSITY HEALTH SCIENCES, WINSTON-SALEM, NC (US)

(72) Inventor: Jan Rohozinski, Houston, TX (US)

(21) Appl. No.: 14/367,695

(22) PCT Filed: Dec. 21, 2012
(86) PCT No.: PCT/US2012/071175

§ 371 Date: Jun. 20, 2014

Related U.S. Application Data
(60) Provisional application No. 61/578,662, filed on Dec. 21, 2011.

(51) Int. Cl. C12Q 1/68

(52) U.S. Cl. CPC C12Q 1/689; C12Q 2600/156; C12Q 2600/112
USPC 506/9; 435/6.11

(57) ABSTRACT

The present invention relates to compositions and methods for using nucleic acid sequences present in the genome of a bacterium to rapidly identify the Gram-stain status of an unknown bacterium present in a biological sample. The invention also relates to compositions and methods for using nucleic acid sequences present in the genome of a bacterium to rapidly determine the species of an unknown bacterium present in a sample.

### CROSS-REFERENCE TO RELATED APPLICATIONS

This application claims the benefit of priority from U.S. Provisional Application Ser. No. 61/578,662, filed on Dec. 21, 2011, the entire disclosure of which is incorporated by reference herein as if set forth herein in its entirety.

### BACKGROUND OF THE INVENTION

The identification of the infectious organism infecting a patient is critical information for enabling a clinician to select the optimal treatment. Historically, the identification of the infectious organism was made by a microbiologist employing methods involving the growth of the organism in the laboratory over several days.

Gram staining is a method used for differentiating bacterial species into two groups called Gram-positive and Gram-negative. Differential staining is dependent on the physical properties and chemistry of the cell walls. In general, Gram-positive bacteria have a thick peptidoglycan cell wall outside of the cell membrane, whereas Gram-negative bacteria have a thin peptidoglycan cell wall sandwiched between the cell membrane and an outer lipopolysaccharide membrane. Gram staining involves four basic steps: primary staining with crystal violet of a heat fixed smear of bacteria on a slide, addition of mordanat (Gram's iodine), rapid decolorization with alcohol or acetone and finally counter staining with safranin or basic fuchsin. The Gram-positive cells retain crystal violet through the decolorization step and appear purple under light microscopy. Gram-negative cells lose the crystal violet, pick up the counter stain and appear pink or red under light microscopy. It is important to know the Gram-stain status of an infectious bacterium to decide on the optimal drug regimen for treatment.

Procedures for detecting and identifying infectious organisms that are resistant to antibiotics are among the most critical tasks performed by clinical and commercial laboratories. Laboratory diagnosis of infectious agents carrying antibiotic resistance is made by experienced microbiologists using methods involving the growth of bacterial isolates on antibiotic containing media and analysis of growth rates. The presence of antibiotic resistance in bacteria that inhabit humans, companion animals and farm animals is becoming a major concern in the realm of human health. One of the most pressing problems is the presence of methicillin resistance (MR) in a broad range of bacteria particularly those contained within, but not limited to, the genus Staphylococcus. This resistance is a major factor affecting clinical outcome in hospitals and surgeries. In addition the gene associated with MR can be transferred across genera and species of bacteria so that MR is becoming an ever increasing problem in hospitals, veterinary medicine and agriculture. MR is most commonly determined by biological methods involving the growth of bacteria on antibiotic containing media.

Microbiologists can now use an extensive array of molecular diagnostic techniques, including the use of real time PCR. The specificity of primer pairs and probes, together with the sensitivity afforded by nucleic acid amplification techniques, has made molecular diagnostics the method for rapidly detecting and identifying infections organisms in biological samples.

There is a need in the art for primer pairs and probes that can rapidly identify and distinguish Gram-positive bacteria from Gram-negative bacteria, rapidly identify and distinguish particular species of bacteria, and rapidly identify and distinguish antibiotic resistant strains of bacteria. There is also a need for the rapid identification of antibiotic resistance in clinical and environmental bacterial samples. The present invention addresses these needs in the art.

### SUMMARY OF THE INVENTION

In various embodiments, the invention relates to compositions and methods of using nucleic acid sequences in the genome of a bacterium to rapidly identify the Gram-stain status of a bacterium present in a biological sample, the species of a bacterium present in a biological sample, or the antibiotic resistance status of a bacterium present in a biological sample.

In one embodiment, the invention is a method of determining the Gram stain status of a bacterium present in a biological sample, including the steps of obtaining a nucleic acid from a bacterium in a biological sample; generating a nucleic acid amplification product of the nucleic acid using a pair of PCR primers (e.g., TACGGGAGGCAGCAGT (SEQ ID NO: 40) and AGCAGCCGCGGTAATA (SEQ ID NO: 41)); and detecting at least one of: (i) the presence or absence of an amplification product that hybridizes to an oligonucleotide probe comprising the sequence: GAGCAACGCCGCGTGA (SEQ ID NO: 42), wherein the presence of the amplification product indicates that the bacteria is a Gram positive; or (ii) the presence or absence of an amplification product that hybridizes to an oligonucleotide probe comprising the sequence: CAGCCATGCCGCGTGT (SEQ ID NO: 43), wherein the presence of the amplification product indicates that the bacteria is Gram negative. In some embodiments, the detecting step comprises detecting both of: (i) the presence or absence of an amplification product that hybridizes to an oligonucleotide probe comprising the sequence: GAGCAACGCCGCGTGA (SEQ ID NO: 42), wherein the presence of the amplification product indicates that the bacteria is a Gram positive and the absence of the amplification product indicates that the bacteria is Gram negative; and (ii) the presence or absence of an amplification product that hybridizes to an oligonucleotide probe comprising the sequence: CAGCCATGCCGCGTGT (SEQ ID NO: 43), wherein the presence of the amplification product indicates that the bacteria is Gram negative and the absence of the amplification product indicates that the bacteria is Gram positive.

In some embodiments, the generating step and the detecting step are carried out concurrently. In other embodiments, the generating step and the detecting step are carried out sequentially. In one embodiment, the generating step and the detecting step are carried out concurrently in a real-time polymerase chain reaction. In some embodiments, the probe selectively binds to the nucleic acid amplification product of a nucleic acid sample of a Gram positive bacteria. In some embodiments, the probe selectively binds to the nucleic acid amplification product of at least one Gram positive bacteria, and does not selectively bind to the nucleic acid amplification product of a nucleic acid sample from a Gram negative bacteria under identical reactions conditions. In various embodiments, the Gram positive bacteria includes at least one of Bacillus species, Lactobacillus species, Micrococcus species, Mycobacterium species, Sporosarcina species, Staphylococcus species, Streptococcus species, Listeria species, Clostridium species, Corynebacterium species, and Enterococcus species. In various embodiments, the Gram positive bacteria includes at least one of Enterococcus faecalis, Enterococcus faecium, Staphylococcus aureus, Staphylococcus aureus (MRSA), Streptococcus pneumoniae, Streptococcus pyogenes, Enterococcus faecalis, Staphylococcus epidermidis, Staphylococcus coagulase, and Streptococcus agalactiae. In some embodiments, the probe selectively binds to the nucleic acid amplification product of a nucleic acid sample of a Gram negative bacteria. In some embodiments, the probe selectively binds to the nucleic acid amplification product of at least one Gram negative bacteria, and does not selectively bind to the nucleic acid amplification product of a nucleic acid sample from a Gram positive bacteria under identical reactions conditions. In various embodiments, the Gram negative bacteria include at least one of Acinetobacter species, Alcaligenes species, Cytophaga species, Enterobacter species, Escherichia species, Liebsiella species, Morganella species, Proteus species, Pseudomonas species, Rhodospirillum species, Salmonella species, Serratia species, Shigella species, Citrobacter species, and Klebsiella species. In various embodiments, the Gram negative bacteria includes at least one of Pseudomonas aeruginosa, Escherichia coli, Acinetobacter baumannii, Enterobacter cloacae, Klebsiella pneumoniae, Proteus mirabilis, Staphylococcus aureus, methicillin resistant Staphylococcus aureus (MRSA), Streptococcus pneumoniae, Streptococcus pyogenes, Enterococcus faecalis, Proteus mirabilis, Klebsiella pneumoniae, Citrobacter koseri, Shigella flexneri, Morganella moranii, Citrobacter freundii, Enterobacter aerogenes, Klebsiella oxytoca, Proteus vulgaris, Providecia stuartii, Pseudomonas flourescens, Pseudomonas putida, Serratia marcescens, Enterobacter sakazakii, Citrobacter youngae, and Klebsiella terrigena.

In another embodiment, the invention is a method of determining the methicillin resistance status of a bacterium present in a biological sample, including the steps of obtaining a nucleic acid from a bacterium in a biological sample; generating a nucleic acid amplification product of the nucleic acid using at least one pair of PCR primers, and detecting the presence or absence of an amplification product that hybridizes to at least one oligonucleotide probe, wherein the presence of the amplification product identifies the bacterium as methicillin resistant. In some embodiments, the generating step and the detecting step are carried out concurrently. In other embodiments, the generating step and the detecting step are carried out sequentially. In one embodiment, the generating step and the detecting step are carried out concurrently in a real-time polymerase chain reaction. In one embodiment, the at least one pair of PCR primers comprises GATGGTATGTGGAAGTTAGATTG (SEQ ID NO: 44) and GACCGAAACAATGTGGAATTGG (SEQ ID NO: 45), and the at least one oligonucleotide probe comprises CATAGCGTCATTATTCCAG (SEQ ID NO: 48). In another embodiment, the at least one pair of PCR primers comprises GATGGTATGTGGAAGTTAGATTG (SEQ ID NO: 44) and GACCGAAACAATGTGGAATTGG (SEQ ID NO: 45), and the at least one oligonucleotide probe comprises CATAGCGTCATTATTCCAGGAATGCAGAA (SEQ ID NO: 49). In another embodiment, the at least one pair of PCR primers comprises GATGGCTATCGTGTCACAATCG (SEQ ID NO: 46) and TTCATATGACGTCTATCCAT (SEQ ID NO: 47), and the at least one oligonucleotide probe comprises GATTATGGCTCAGGTACTGCTATC (SEQ ID NO: 50). In yet another embodiment, the at least one pair of PCR primers comprises GATAAGCATTGGAAATTAGATTG (SEQ ID NO: 80) and AGCTAATTCTATATTGTTTCGGTC (SEQ ID NO: 81), and the at least one oligonucleotide probe comprises CCAGACGTAATAGTACCTG (SEQ ID NO: 82).

In one embodiment, the invention is a method of identifying a bacterium present in a biological sample, including the steps of obtaining a nucleic acid from a bacterium in a biological sample; generating a nucleic acid amplification product of the nucleic acid using at least one pair of PCR primers; and detecting the presence or absence of an amplification product that hybridizes to at least one oligonucleotide probe, wherein binding of the at least one oligonucleotide probe to the amplification product identifies the bacterium present in the sample. In one embodiment, the generating step and the detecting step are carried out concurrently. In another embodiment, the generating step and the detecting step are carried out sequentially. In some embodiments, the generating step and the detecting step are carried out concurrently in a real-time polymerase chain reaction. In one embodiment, the at least one pair of PCR primers comprises AGAGTTTGATCHTGGCTCAG (SEQ ID NO: 83) and CCYACTGCTGCCTCCCGTA (SEQ ID NO: 84). In another embodiment, the at least one pair of PCR primers comprises GGGAHGAMGWCAARTCATCAT (SEQ ID NO: 85) and CGATTACTAGCGATTCCRRCTTC (SEQ ID NO: 86). In yet another embodiment, the at least one pair of PCR primers comprises GAAGYYGGAATCGCTAGTAATCG (SEQ ID NO: 87) and TACRGHTACCTTGTTACGACT (SEQ ID NO: 88). In various embodiments, the at least one oligonucleotide probe is at least one selected from the group consisting of GAAAACAATGGCGCA (SEQ ID NO: 89), ACGGTTACCACGGAG (SEQ ID NO: 90), CTAGCCTAACTGCAAAGA (SEQ ID NO: 91), GATGACCGCCACACT (SEQ ID NO: 92), GCCTCTTGCCATCA (SEQ ID NO: 93), GCCTCATGCCATCA (SEQ ID NO: 94), GGCAGATACAAAGAG (SEQ ID NO: 95), GGCGTATACAAAGGG (SEQ ID NO: 96), GATCTGCCTGATGGC (SEQ ID NO: 97), AACGAGTCGCTAGAC (SEQ ID NO: 98), AACGAGTTGCGAAGT (SEQ ID NO: 99), ACCGTAAGGAGCCAG (SEQ ID NO: 100), CCTATTAGGAGCCAG (SEQ ID NO: 101), TTCTCTGATGTTAGC (SEQ ID NO: 102), TCCTCTGACGTTAGC (SEQ ID NO: 103), CAACGTTTCCAAAGGA (SEQ ID NO: 104), TAGCACAGAGGAGCTT (SEQ ID NO: 105), TAGCACAGAGGAGGCTT (SEQ ID NO: 106), CAGCTTGCTGCTTCGCT (SEQ ID NO: 107), TTGTAGGTGAGGTAAC (SEQ ID NO: 108), TAGCACAAGGGAGCTTG (SEQ ID NO: 109), GGACCCGCGCCGTATT (SEQ ID NO: 110), ATCTCTTAGGAGCAAAGC (SEQ ID NO: 111), and CTGAGGTTTGGTGTTTA (SEQ ID NO: 112).

In another embodiment, the invention is a method of determining whether a bacterium present in a biological sample is Staphylococcus aureus, including the steps of obtaining a nucleic acid from a bacterium in a biological sample; generating a nucleic acid amplification product of the nucleic acid using a pair of PCR primers, wherein the pair of PCR primers comprises AGAGTTTGATCHTGGCTCAG (SEQ ID NO: 51) and CCTACTGCTGCCTCCCTGTA (SEQ ID NO: 52); and detecting the presence or absence of an amplification product that hybridizes to an oligonucleotide probe comprising the sequence: TTCTCTGATGTTAGC (SEQ ID NO: 53), the presence of the amplification product indicating the bacteria is Staphylococcus aureus. In one embodiment, the generating step and the detecting step are carried out concurrently. In another embodiment, the generating step and the detecting step are carried out sequentially. In some embodiments, the generating step and the detecting step are carried out concurrently in a real-time polymerase chain reaction.

In one embodiment, the invention is a method of determining whether a bacterium present in a biological sample is Staphylococcus epidermidis, including the steps of obtaining a nucleic acid from a bacterium in a biological sample; generating a nucleic acid amplification product of the nucleic acid using a pair of PCR primers, wherein the pair of PCR primers comprises AGAGTTTGATCHTGGCTCAG (SEQ ID NO: 54) and CCTACTGCTGCCTCCCTGTA (SEQ ID NO: 55); and detecting the presence or absence of an amplification product that hybridizes to an oligonucleotide probe comprising the sequence: TCCTCTGACGTTAGC (SEQ ID NO: 56), the presence of the amplification product indicating the bacteria is Staphylococcus epidermidis. In one embodiment, the generating step and the detecting step are carried out concurrently. In another embodiment, the generating step and the detecting step are carried out sequentially. In some embodiments, the generating step and the detecting step are carried out concurrently in a real-time polymerase chain reaction.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 shows the real time PCR curves generated from the nucleic acid of gram negative Pseudomonas aeruginosa for the detection of gram negative bacteria (left), using SEQ ID NO: 43 as the probe, and for the detection of gram positive bacteria (right), using SEQ ID NO: 42 as the probe.

FIG. 2 shows the real time PCR curves generated from the nucleic acid of gram negative Escherichia coli for the detection of gram negative bacteria (left), using SEQ ID NO: 43 as the probe, and for the detection of gram positive bacteria (right), using SEQ ID NO: 42 as the probe.

FIG. 3 shows the real time PCR curves generated from the nucleic acid of gram negative Shigella flexneri for the detection of gram negative bacteria (left), using SEQ ID NO: 43 as the probe, and for the detection of gram positive bacteria (right), using SEQ ID NO: 42 as the probe.

FIG. 4 shows the real time PCR curves generated from the nucleic acid of gram negative Morganella moranii for the detection of gram negative bacteria (left), using SEQ ID NO: 43 as the probe, and for the detection of gram positive bacteria (right), using SEQ ID NO: 42 as the probe.

FIG. 5 shows the real time PCR curves generated from the nucleic acid of gram negative Citrobacter freundii for the detection of gram negative bacteria (left), using SEQ ID NO: 43 as the probe, and for the detection of gram positive bacteria (right), using SEQ ID NO: 42 as the probe.

FIG. 6 shows the real time PCR curves generated from the nucleic acid of gram negative Enterobacter aerogenes for the detection of gram negative bacteria (left), using SEQ ID NO: 43 as the probe, and for the detection of gram positive bacteria (right), using SEQ ID NO: 42 as the probe.

FIG. 7 shows the real time PCR curves generated from the nucleic acid of gram negative Klebsilla oxytoca for the detection of gram negative bacteria (left), using SEQ ID NO: 43 as the probe, and for the detection of gram positive bacteria (right), using SEQ ID NO: 42 as the probe.

FIG. 8 shows the real time PCR curves generated from the nucleic acid of gram negative Proteus vulgaris for the detection of gram negative bacteria (left), using SEQ ID NO: 43 as the probe, and for the detection of gram positive bacteria (right), using SEQ ID NO: 42 as the probe.

FIG. 9 shows the real time PCR curves generated from the nucleic acid of gram negative Providecia stuartii for the detection of gram negative bacteria (left), using SEQ ID NO: 43 as the probe, and for the detection of gram positive bacteria (right), using SEQ ID NO: 42 as the probe.

FIG. 10 shows the real time PCR curves generated from the nucleic acid of gram negative Pseudomonas fluorescens for the detection of gram negative bacteria (left), using SEQ ID NO: 43 as the probe, and for the detection of gram positive bacteria (right), using SEQ ID NO: 42 as the probe.

FIG. 11 shows the real time PCR curves generated from the nucleic acid of gram negative Pseudomonas putida for the detection of gram negative bacteria (left), using SEQ ID NO: 43 as the probe, and for the detection of gram positive bacteria (right), using SEQ ID NO: 42 as the probe.

FIG. 12 shows the real time PCR curves generated from the nucleic acid of gram negative Serratia marcescens for the detection of gram negative bacteria (left), using SEQ ID NO: 43 as the probe, and for the detection of gram positive bacteria (right), using SEQ ID NO: 42 as the probe.

FIG. 13 shows the real time PCR curves generated from the nucleic acid of gram negative Enterobacter sakazakii for the detection of gram negative bacteria (left), using SEQ ID NO: 43 as the probe, and for the detection of gram positive bacteria (right), using SEQ ID NO: 42 as the probe.

FIG. 14 shows the real time PCR curves generated from the nucleic acid of gram negative Citrobacter youngae for the detection of gram negative bacteria (left), using SEQ ID NO: 43 as the probe, and for the detection of gram positive bacteria (right), using SEQ ID NO: 42 as the probe.

FIG. 15 shows the real time PCR curves generated from the nucleic acid of gram negative Klebsiella terrigena for the detection of gram negative bacteria (left), using SEQ ID NO: 43 as the probe, and for the detection of gram positive bacteria (right), using SEQ ID NO: 42 as the probe.

FIG. 16 shows the real time PCR curves generated from the nucleic acid of gram negative Acinetobacter baumannii for the detection of gram negative bacteria (left), using SEQ ID NO: 43 as the probe, and for the detection of gram positive bacteria (right), using SEQ ID NO: 42 as the probe.

FIG. 17 shows the real time PCR curves generated from the nucleic acid of gram negative Enterobacter cloacae for the detection of gram negative bacteria (left), using SEQ ID NO: 43 as the probe, and for the detection of gram positive bacteria (right), using SEQ ID NO: 42 as the probe.

FIG. 18 shows the real time PCR curves generated from the nucleic acid of gram negative Klebsiella pneumoniae for the detection of gram negative bacteria (left), using SEQ ID NO: 43 as the probe, and for the detection of gram positive bacteria (right), using SEQ ID NO: 42 as the probe.

FIG. 19 shows the real time PCR curves generated from the nucleic acid of gram negative Proteus mirabilis for the detection of gram negative bacteria (left), using SEQ ID NO: 43 as the probe, and for the detection of gram positive bacteria (right), using SEQ ID NO: 42 as the probe.

FIG. 20 shows the real time PCR curves generated from the nucleic acid of gram negative Citrobacter koseri for the detection of gram negative bacteria (left), using SEQ ID NO: 43 as the probe, and for the detection of gram positive bacteria (right), using SEQ ID NO: 42 as the probe.

FIG. 21 shows the real time PCR curves generated from the nucleic acid of gram positive Staphylococcus aureus for the detection of gram negative bacteria (left), using SEQ ID NO: 43 as the probe, and for the detection of gram positive bacteria (right), using SEQ ID NO: 42 as the probe.

FIG. 22 shows the real time PCR curves generated from the nucleic acid of gram positive Streptococcus pneumoniae for the detection of gram negative bacteria (left), using SEQ ID NO: 43 as the probe, and for the detection of gram positive bacteria (right), using SEQ ID NO: 42 as the probe.

FIG. 23 shows the real time PCR curves generated from the nucleic acid of gram positive methicillin-resistant Staphylococcus aureus for the detection of gram negative bacteria (left), using SEQ ID NO: 43 as the probe, and for the detection of gram positive bacteria (right), using SEQ ID NO: 42 as the probe.

FIG. 24 shows the real time PCR curves generated from the nucleic acid of gram positive methicillin-resistant Staphylococcus epidermidis for the detection of gram negative bacteria (left), using SEQ ID NO: 43 as the probe, and for the detection of gram positive bacteria (right), using SEQ ID NO: 42 as the probe.

FIG. 25 shows the real time PCR curves generated from the nucleic acid of gram positive methicillin-resistant Staphylococcus intermedius for the detection of gram negative bacteria (left), using SEQ ID NO: 43 as the probe, and for the detection of gram positive bacteria (right), using SEQ ID NO: 42 as the probe.

FIG. 26 shows the real time PCR curves generated from the nucleic acid of gram positive methicillin-susceptible Staphylococcus aureus for the detection of gram negative bacteria (left), using SEQ ID NO: 43 as the probe, and for the detection of gram positive bacteria (right), using SEQ ID NO: 42 as the probe.

FIG. 27 shows the real time PCR curves generated from the nucleic acid of gram positive Staphylococcus haemolyticus for the detection of gram negative bacteria (left), using SEQ ID NO: 43 as the probe, and for the detection of gram positive bacteria (right), using SEQ ID NO: 42 as the probe.

FIG. 28 shows the real time PCR curves generated from the nucleic acid of gram positive Staphylococcus pseudintermedius for the detection of gram negative bacteria (left), using SEQ ID NO: 43 as the probe, and for the detection of gram positive bacteria (right), using SEQ ID NO: 42 as the probe.

FIG. 29 shows the real time PCR curves generated from the nucleic acid of gram positive Staphylococcus schleiferi for the detection of gram negative bacteria (left), using SEQ ID NO: 43 as the probe, and for the detection of gram positive bacteria (right), using SEQ ID NO: 42 as the probe.

FIG. 30 shows the real time PCR curves generated from the nucleic acid of gram positive Staphylococcus warneri for the detection of gram negative bacteria (left), using SEQ ID NO: 43 as the probe, and for the detection of gram positive bacteria (right), using SEQ ID NO: 42 as the probe.

FIG. 31 shows the real time PCR curves generated from the nucleic acid of gram positive Enterococcus faecalis for the detection of gram negative bacteria (left), using SEQ ID NO: 43 as the probe, and for the detection of gram positive bacteria (right), using SEQ ID NO: 42 as the probe.

FIG. 32 shows the real time PCR curves generated from the nucleic acid of gram positive Enterococcus faecium for the detection of gram negative bacteria (left), using SEQ ID NO: 43 as the probe, and for the detection of gram positive bacteria (right), using SEQ ID NO: 42 as the probe.

FIG. 33 shows the real time PCR curves generated from the nucleic acid of gram positive Staphylococcus pyrogenes for the detection of gram negative bacteria (left), using SEQ ID NO: 43 as the probe, and for the detection of gram positive bacteria (right), using SEQ ID NO: 42 as the probe.

FIG. 34 shows the real time PCR curves generated from the nucleic acid of gram positive Staphylococcus lugdunensis for the detection of gram negative bacteria (left), using SEQ ID NO: 43 as the probe, and for the detection of gram positive bacteria (right), using SEQ ID NO: 42 as the probe.

FIG. 35 shows the real time PCR curves generated from the nucleic acid of gram positive Streptococcus agalactiae for the detection of gram negative bacteria (left), using SEQ ID NO: 43 as the probe, and for the detection of gram positive bacteria (right), using SEQ ID NO: 42 as the probe.

FIG. 36 shows the real time PCR curve generated from the nucleic acid of Shigella flexneri for the detection of methicillin resistance using SEQ ID NO: 48 as the probe.

FIG. 37 shows the real time PCR curve generated from the nucleic acid of Morganella moranii for the detection of methicillin resistance using SEQ ID NO: 48 as the probe.

FIG. 38 shows the real time PCR curve generated from the nucleic acid of Citrobacter freundii for the detection of methicillin resistance using SEQ ID NO: 48 as the probe.

FIG. 39 shows the real time PCR curve generated from the nucleic acid of Enterobacter aerogenes for the detection of methicillin resistance using SEQ ID NO: 48 as the probe.

FIG. 40 shows the real time PCR curve generated from the nucleic acid of Klebsiella oxytoca for the detection of methicillin resistance using SEQ ID NO: 48 as the probe.

FIG. 41 shows the real time PCR curve generated from the nucleic acid of Proteus vulgaris for the detection of methicillin resistance using SEQ ID NO: 48 as the probe.

FIG. 42 shows the real time PCR curve generated from the nucleic acid of Providecia stuartii for the detection of methicillin resistance using SEQ ID NO: 48 as the probe.

FIG. 43 shows the real time PCR curve generated from the nucleic acid of Pseudomonas fluorescens for the detection of methicillin resistance using SEQ ID NO: 48 as the probe.

FIG. 44 shows the real time PCR curve generated from the nucleic acid of Psudomonas putida for the detection of methicillin resistance using SEQ ID NO: 48 as the probe.

FIG. 45 shows the real time PCR curve generated from the nucleic acid of Serratia marcescens for the detection of methicillin resistance using SEQ ID NO: 48 as the probe.

FIG. 46 shows the real time PCR curve generated from the nucleic acid of Enterobacter sakazakii for the detection of methicillin resistance using SEQ ID NO: 48 as the probe.

FIG. 47 shows the real time PCR curve generated from the nucleic acid of Citrobacter youngae for the detection of methicillin resistance using SEQ ID NO: 48 as the probe.

FIG. 48 shows the real time PCR curve generated from the nucleic acid of Klebsiella terrigena for the detection of methicillin resistance using SEQ ID NO: 48 as the probe.

FIG. 49 shows the real time PCR curve generated from the nucleic acid of Psudomonas aeruginosa for the detection of methicillin resistance using SEQ ID NO: 48 as the probe.

FIG. 50 shows the real time PCR curve generated from the nucleic acid of Staphylococcus aureus (MRSA) for the detection of methicillin resistance using SEQ ID NO: 48 as the probe.

FIG. 51 shows the real time PCR curve generated from the nucleic acid of Streptococcus pneumoniae for the detection of methicillin resistance using SEQ ID NO: 48 as the probe.

FIG. 52 shows the real time PCR curve generated from the nucleic acid of Streptococcus pyrogenes for the detection of methicillin resistance using SEQ ID NO: 48 as the probe.

FIG. 53 shows the real time PCR curve generated from the nucleic acid of Escherichia coli for the detection of methicillin resistance using SEQ ID NO: 48 as the probe.

FIG. 54 shows the real time PCR curve generated from the nucleic acid of methicillin-resistant Staphylococcus haemolyticus for the detection of methicillin resistance using SEQ ID NO: 48 as the probe.

FIG. 55 shows the real time PCR curve generated from the nucleic acid of methicillin-resistant Staphylococcus schleiferi for the detection of methicillin resistance using SEQ ID NO: 48 as the probe.

FIG. 56 shows the real time PCR curve generated from the nucleic acid of Staphylococcus pseudintermedius for the detection of methicillin resistance using SEQ ID NO: 48 as the probe.

FIG. 57 shows the real time PCR curve generated from the nucleic acid of Staphylococcus warneri for the detection of methicillin resistance using SEQ ID NO: 48 as the probe.

FIG. 58 shows the real time PCR curve generated from the nucleic acid of methicillin-susceptible Staphylococcus aureus for the detection of methicillin resistance using SEQ ID NO: 48 as the probe.

FIG. 59 shows the real time PCR curve generated from the nucleic acid of methicillin-resistant Staphylococcus aureus for the detection of methicillin resistance using SEQ ID NO: 48 as the probe.

FIG. 60 shows the real time PCR curve generated from the nucleic acid of methicillin-resistant Staphylococcus intermedius for the detection of methicillin resistance using SEQ ID NO: 48 as the probe.

FIG. 61 shows the real time PCR curve generated from the nucleic acid of methicillin-resistant Staphylococcus epidermidis for the detection of methicillin resistance using SEQ ID NO: 48 as the probe.

FIG. 62 shows the real time PCR curve generated from the nucleic acid of Acintobacter baumanni for the detection of Staphylococcus aureus, using SEQ ID NO: 53 as the probe.

FIG. 63 shows the real time PCR curve generated from the nucleic acid of Enterobacter cloacae for the detection of Staphylococcus aureus, using SEQ ID NO: 53 as the probe.

FIG. 64 shows the real time PCR curve generated from the nucleic acid of Enterococcus faecalis for the detection of Staphylococcus aureus, using SEQ ID NO: 53 as the probe.

FIG. 65 shows the real time PCR curve generated from the nucleic acid of Enterococcus faecium for the detection of Staphylococcus aureus, using SEQ ID NO: 53 as the probe.

FIG. 66 shows the real time PCR curve generated from the nucleic acid of Klebsiella pneumoniae for the detection of Staphylococcus aureus, using SEQ ID NO: 53 as the probe.

FIG. 67 shows the real time PCR curve generated from the nucleic acid of Pseudomonas aeruginosa for the detection of Staphylococcus aureus, using SEQ ID NO: 53 as the probe.

FIG. 68 shows the real time PCR curve generated from the nucleic acid of Proteus mirabilis for the detection of Staphylococcus aureus, using SEQ ID NO: 53 as the probe.

FIG. 69 shows the real time PCR curve generated from the nucleic acid of Staphylococcus aureus for the detection of Staphylococcus aureus, using SEQ ID NO: 53 as the probe.

FIG. 70 shows the real time PCR curve generated from the nucleic acid of Streptococcus pneumoniae for the detection of Staphylococcus aureus, using SEQ ID NO: 53 as the probe.

FIG. 71 shows the real time PCR curve generated from the nucleic acid of Streptococcus pyogenes for the detection of Staphylococcus aureus, using SEQ ID NO: 53 as the probe.

FIG. 72 shows the real time PCR curve generated from the nucleic acid of Staphylococcus lugdunensis for the detection of Staphylococcus aureus, using SEQ ID NO: 53 as the probe.

FIG. 73 shows the real time PCR curve generated from the nucleic acid of Streptococcus agalactiae for the detection of Staphylococcus aureus, using SEQ ID NO: 53 as the probe.

FIG. 74 shows the real time PCR curve generated from the nucleic acid of Staphylococcus haemolyticus for the detection of Staphylococcus aureus, using SEQ ID NO: 53 as the probe.

FIG. 75 shows the real time PCR curve generated from the nucleic acid of Escherichia coli for the detection of Staphylococcus aureus, using SEQ ID NO: 53 as the probe.

FIG. 76 shows the real time PCR curve generated from the nucleic acid of Staphylococcus schleiferi for the detection of Staphylococcus aureus, using SEQ ID NO: 53 as the probe.

FIG. 77 shows the real time PCR curve generated from the nucleic acid of Staphylococcus pseudintermedius for the detection of Staphylococcus aureus, using SEQ ID NO: 53 as the probe.

FIG. 78 shows the real time PCR curve generated from the nucleic acid of methicillin-resistant Staphylococcus intermedius for the detection of Staphylococcus aureus, using SEQ ID NO: 53 as the probe.

FIG. 79 shows the real time PCR curve generated from the nucleic acid of methicillin-resistant Staphylococcus epidermidis for the detection of Staphylococcus aureus, using SEQ ID NO: 53 as the probe.

FIG. 80 shows the real time PCR curve generated from the nucleic acid of methicillin-susceptible Staphylococcus aureus for the detection of Staphylococcus aureus, using SEQ ID NO: 53 as the probe.

FIG. 81 shows the real time PCR curve generated from the nucleic acid of methicillin-resistant Staphylococcus aureus for the detection of Staphylococcus aureus, using SEQ ID NO: 53 as the probe.

FIG. 82 shows the real time PCR curve generated from the nucleic acid of Shigella flexneri for the detection of Staphylococcus aureus, using SEQ ID NO: 53 as the probe.

FIG. 83 shows the real time PCR curve generated from the nucleic acid of Morganella moranii for the detection of Staphylococcus aureus, using SEQ ID NO: 53 as the probe.

FIG. 84 shows the real time PCR curve generated from the nucleic acid of Citrobacter freundii for the detection of Staphylococcus aureus, using SEQ ID NO: 53 as the probe.

FIG. 85 shows the real time PCR curve generated from the nucleic acid of Enterobacter aerogenes for the detection of Staphylococcus aureus, using SEQ ID NO: 53 as the probe.

FIG. 86 shows the real time PCR curve generated from the nucleic acid of Klebsiella oxytoca for the detection of Staphylococcus aureus, using SEQ ID NO: 53 as the probe.

FIG. 87 shows the real time PCR curve generated from the nucleic acid of Proteus vulgaris for the detection of Staphylococcus aureus, using SEQ ID NO: 53 as the probe.

FIG. 88 shows the real time PCR curve generated from the nucleic acid of Providecia stuartii for the detection of Staphylococcus aureus, using SEQ ID NO: 53 as the probe.

FIG. 89 shows the real time PCR curve generated from the nucleic acid of Pseudomonas fluorescens for the detection of Staphylococcus aureus, using SEQ ID NO: 53 as the probe.

FIG. 90 shows the real time PCR curve generated from the nucleic acid of Pseudomonas putida for the detection of Staphylococcus aureus, using SEQ ID NO: 53 as the probe.

FIG. 91 shows the real time PCR curve generated from the nucleic acid of Serratia marcescens for the detection of Staphylococcus aureus, using SEQ ID NO: 53 as the probe.

FIG. 92 shows the real time PCR curve generated from the nucleic acid of Staphylococcus epidermidis for the detection of Staphylococcus aureus, using SEQ ID NO: 53 as the probe.

FIG. 93 shows the real time PCR curve generated from the nucleic acid of Enterobacter sakazakii for the detection of Staphylococcus aureus, using SEQ ID NO: 53 as the probe.

FIG. 94 shows the real time PCR curve generated from the nucleic acid of Citrobacter youngae for the detection of Staphylococcus aureus, using SEQ ID NO: 53 as the probe.

FIG. 95 shows the real time PCR curve generated from the nucleic acid of Klebsiella terrigena for the detection of Staphylococcus aureus, using SEQ ID NO: 53 as the probe.

FIG. 96 shows the real time PCR curve generated from the nucleic acid of Acintobacter baumannii for the detection of Staphylococcus epidermidis, using SEQ ID NO: 56 as the probe.

FIG. 97 shows the real time PCR curve generated from the nucleic acid of Enterobacter cloacae for the detection of Staphylococcus epidermidis, using SEQ ID NO: 56 as the probe.

FIG. 98 shows the real time PCR curve generated from the nucleic acid of Enterococcus faecalis for the detection of Staphylococcus epidermidis, using SEQ ID NO: 56 as the probe.

FIG. 99 shows the real time PCR curve generated from the nucleic acid of Enterococcus faecium for the detection of Staphylococcus epidermidis, using SEQ ID NO: 56 as the probe.

FIG. 100 shows the real time PCR curve generated from the nucleic acid of Klebsiella pneumoniae for the detection of Staphylococcus epidermidis, using SEQ ID NO: 56 as the probe.

FIG. 101 shows the real time PCR curve generated from the nucleic acid of Pseudomonas aeruginosa for the detection of Staphylococcus epidermidis, using SEQ ID NO: 56 as the probe.

FIG. 102 shows the real time PCR curve generated from the nucleic acid of Proteus mirabilis for the detection of Staphylococcus epidermidis, using SEQ ID NO: 56 as the probe.

FIG. 103 shows the real time PCR curve generated from the nucleic acid of Staphylococcus aureus for the detection of Staphylococcus epidermidis, using SEQ ID NO: 56 as the probe.

FIG. 104 shows the real time PCR curve generated from the nucleic acid of Streptococcus pneumoniae for the detection of Staphylococcus epidermidis, using SEQ ID NO: 56 as the probe.

FIG. 105 shows the real time PCR curve generated from the nucleic acid of Streptococcus pyogenes for the detection of Staphylococcus epidermidis, using SEQ ID NO: 56 as the probe.

FIG. 106 shows the real time PCR curve generated from the nucleic acid of Shigella flexneri for the detection of Staphylococcus epidermidis, using SEQ ID NO: 56 as the probe.

FIG. 107 shows the real time PCR curve generated from the nucleic acid of Morganella moranii for the detection of Staphylococcus epidermidis, using SEQ ID NO: 56 as the probe.

FIG. 108 shows the real time PCR curve generated from the nucleic acid of Citrobacter freundii for the detection of Staphylococcus epidermidis, using SEQ ID NO: 56 as the probe.

FIG. 109 shows the real time PCR curve generated from the nucleic acid of Enterobacter aerogenes for the detection of Staphylococcus epidermidis, using SEQ ID NO: 56 as the probe.

FIG. 110 shows the real time PCR curve generated from the nucleic acid of Klebsiella oxytoca for the detection of Staphylococcus epidermidis, using SEQ ID NO: 56 as the probe.

FIG. 111 shows the real time PCR curve generated from the nucleic acid of Proteus vulgaris for the detection of Staphylococcus epidermidis, using SEQ ID NO: 56 as the probe.

FIG. 112 shows the real time PCR curve generated from the nucleic acid of Providecia stuartii for the detection of Staphylococcus epidermidis, using SEQ ID NO: 56 as the probe.

FIG. 113 shows the real time PCR curve generated from the nucleic acid of Pseudomonas fluorescens for the detection of Staphylococcus epidermidis, using SEQ ID NO: 56 as the probe.

FIG. 114 shows the real time PCR curve generated from the nucleic acid of Psuedomonas putida for the detection of Staphylococcus epidermidis, using SEQ ID NO: 56 as the probe.

FIG. 115 shows the real time PCR curve generated from the nucleic acid of Serratia marcescens for the detection of Staphylococcus epidermidis, using SEQ ID NO: 56 as the probe.

FIG. 116 shows the real time PCR curve generated from the nucleic acid of Staphylococcus epidermidis for the detection of Staphylococcus epidermidis, using SEQ ID NO: 56 as the probe.

FIG. 117 shows the real time PCR curve generated from the nucleic acid of Enterobacter sakazakii for the detection of Staphylococcus epidermidis, using SEQ ID NO: 56 as the probe.

FIG. 118 shows the real time PCR curve generated from the nucleic acid of Citrobacter youngae for the detection of Staphylococcus epidermidis, using SEQ ID NO: 56 as the probe.

FIG. 119 shows the real time PCR curve generated from the nucleic acid of Klebsiella terrigena for the detection of Staphylococcus epidermidis, using SEQ ID NO: 56 as the probe.

FIG. 120 shows the real time PCR curve generated from the nucleic acid of Escherichia coli for the detection of Staphylococcus epidermidis, using SEQ ID NO: 56 as the probe.

FIG. 121 shows the real time PCR curve generated from the nucleic acid of Staphylococcus haemolyticus for the detection of Staphylococcus epidermidis, using SEQ ID NO: 56 as the probe.

FIG. 122 shows the real time PCR curve generated from the nucleic acid of Staphylococcus pseudintermedius for the detection of Staphylococcus epidermidis, using SEQ ID NO: 56 as the probe.

FIG. 123 shows the real time PCR curve generated from the nucleic acid of Staphylococcus schleiferi for the detection of Staphylococcus epidermidis, using SEQ ID NO: 56 as the probe.

FIG. 124 shows the real time PCR curve generated from the nucleic acid of Streptococcus agalactiae for the detection of Staphylococcus epidermidis, using SEQ ID NO: 56 as the probe.

FIG. 125 shows the real time PCR curve generated from the nucleic acid of Staphylococcus lugdunensis for the detection of Staphylococcus epidermidis, using SEQ ID NO: 56 as the probe.

FIG. 126 shows the real time PCR curve generated from the nucleic acid of methicillin-resistant Staphylococcus aureus for the detection of Staphylococcus epidermidis, using SEQ ID NO: 56 as the probe.

FIG. 127 shows the real time PCR curve generated from the nucleic acid of methicillin-resistant Staphylococcus Epidermidis for the detection of Staphylococcus epidermidis, using SEQ ID NO: 56 as the probe.

FIG. 128 shows the real time PCR curve generated from the nucleic acid of methicillin-resistant Staphylococcus intermedius for the detection of Staphylococcus epidermidis, using SEQ ID NO: 56 as the probe.

FIG. 129 shows the real time PCR curve generated from the nucleic acid of methicillin-susceptible Staphylococcus aureus for the detection of Staphylococcus epidermidis, using SEQ ID NO: 56 as the probe.

### DETAILED DESCRIPTION

The present invention relates to compositions and methods for using nucleic acid sequences in the genome of a bacterium to rapidly identify the Gram-stain status of an unknown bacterium present in a biological sample. The invention also relates to compositions and methods for using nucleic acid sequences in the genome of bacterium to rapidly determine the species of an unknown bacterium present in a biological sample. Further, the invention relates to compositions and methods for using nucleic acid sequences in the genome of bacterium to rapidly determine whether an unknown bacterium present in a biological sample is resistant to a particular antibiotic. Combinations of any of the methods described herein can be performed concurrently or sequential to determine multiple characteristics of an unknown bacterium present in a biological sample.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “adjacent” is used to refer to nucleotide sequences which are directly attached to one another, having no intervening nucleotides. By way of example, the pentanucleotide 5′-AAAAA-3′ is adjacent the trinucleotide 5′-TTT-3′ when the two are connected thus: 5′-AAAAATTT-3′ or 5′-TTTAAAAA-3′, but not when the two are connected thus: 5′-AAAAACTTT-3′.

“Amplification” refers to any means by which a polynucleotide sequence is copied and thus expanded into a larger number of polynucleotide molecules, e.g., by reverse transcription, polymerase chain reaction, and ligase chain reaction, among others.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “microarray” refers broadly to both “DNA microarrays” and “DNA chip(s),” and encompasses all art-recognized solid supports, such as, but not limited to, glass, plastic, or synthetic membrane, and all art-recognized methods for affixing nucleic acid molecules thereto or for synthesis of nucleic acids thereon. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “isolate” refers to a nucleic acid obtained from an individual, or from a sample obtained from an individual. The nucleic acid may be analyzed at any time after it is obtained (e.g., before or after laboratory culture, before or after amplification.)

As used herein, “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3ATTGCC5′ and 3TATGGC share 50% homology.

As used herein, “homology” is used synonymously with “identity.” In addition, when the term “homology” is used herein to refer to the nucleic acids and proteins, it should be construed to be applied to homology at both the nucleic acid and the amino acid levels. The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. MoI. Biol. 215:403-410), and can be accessed, for example, at the National Center for Biotechnology Information (NCBI) world wide web site having the universal resource locator www.ncbi.nlm.nih.gov/BLAST/. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See www<dot>ncbi<dot>nlm<dot>nih<dot>gov. The percent identity between two sequences can be determined using techniques similar to those described above, with or without allowing gaps. In calculating percent identity, typically exact matches are counted.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, e.g., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with a detectable label, e.g., chromogenic, radioactive, or fluorescent moieties and used as detectable moieties. Examples of fluorescent moieties include, but are not limited to, rare earth chelates (europium chelates), Texas Red, rhodamine, fluorescein, dansyl, phycocrytherin, phycocyanin, spectrum orange, spectrum green, and/or derivatives of any one or more of the above. Other detectable moieties include digoxigenin and biotin.

A third primer is “nested” with respect to a first primer and a second primer if amplification of a region of a first oligonucleotide using the first primer and the second primer yields a second oligonucleotide, wherein the third primer is complementary to a portion of the second oligonucleotide, wherein the portion of the second oligonucleotide does not include a nucleotide residue at an end of the second oligonucleotide.

As used herein a “probe” is defined as a nucleic acid capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, a linkage other than a phosphodiester bond may join the bases in probes, so long as it does not interfere with hybridization. Thus, probes may be peptide nucleic acids in which the constituent bases are joined by peptide bonds rather than phosphodiester linkages. The term “match,” “perfect match,” “perfect match probe” or “perfect match control” refers to a nucleic acid that has a sequence that is perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.”

A first oligonucleotide anneals with a second oligonucleotide with “high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g. Sambrook et al., 2001, Molecular Cloning: A Laboratory Manual, Cold Spring Harbor Laboratory Press, Cold Spring Harbor, N.Y.).

A “restriction site” is a portion of a double-stranded nucleic acid which is recognized by a restriction endonuclease. A portion of a double-stranded nucleic acid is “recognized” by a restriction endonuclease if the endonuclease is capable of cleaving both strands of the nucleic acid at a specific location in the portion when the nucleic acid and the endonuclease are contacted. Restriction endonucleases, their cognate recognition sites and cleavage sites are well known in the art. See, for instance, Roberts et al., 2005, Nucleic Acids Research 33:D230-D232.

The term “mismatch,” “mismatch control” or “mismatch probe” refers to a nucleic acid whose sequence is not perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

A “homo-mismatch” substitutes an adenine (A) for a thymine (T) and vice versa and a guanine (G) for a cytosine (C) and vice versa. For example, if the target sequence was: AGGTCCA, a probe designed with a single homo-mismatch at the central, or fourth position, would result in the following sequence: TCCTGGT. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” In the sequences described herein, A=adenine, G=guanine, T=thymine, C=cytosine, H=A, C or T, R=A or G, M=A or C, Y=C or T, W=A or T. The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

A “genome” is all the genetic material of an organism. The term genome may refer to genetic materials from organisms that have or that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variants,” “polymorphisms,” or “mutations.”

Polymorphism refers to the occurrence of two or more genetically determined alternative sequences or alleles in a population. A polymorphic marker or site is the locus at which divergence occurs. A polymorphism may comprise one or more base changes, an insertion, a repeat, or a deletion. A polymorphic locus may be as small as one base pair. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (about at least 1%) in a given population. A SNP may arise due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

The term “genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. A polymorphic location may have two or more possible alleles and the array may be designed to distinguish between all possible combinations.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

“Hybridization probes” are oligonucleotides capable of binding in a base-specific manner to a complementary strand of nucleic acid. Such probes include peptide nucleic acids, as described in Nielsen et al., 1991, Science 254, 1497-1500, and other nucleic acid analogs and nucleic acid mimetics. See U.S. Pat. No. 6,156,501.

The term “hybridization” refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example, two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

The term “label” as used herein refers to a luminescent label, a light scattering label or a radioactive label. Fluorescent labels include, but are not limited to, the commercially available fluorescein phosphoramidites such as Fluoreprime (Pharmacia), Fluoredite (Millipore) and FAM (ABI). See U.S. Pat. No. 6,287,778.

The term “solid support”, “support”, and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, oligonucleotides and nucleic acids. Targets are sometimes referred to in the art as anti-probes.

A “probe target pair” is formed when two macromolecules have combined through molecular recognition to form a complex.

### DESCRIPTION

The compositions and methods of the invention employ nucleic acid probes and primers useful for the amplification and identification of sequences in the genome of a bacterium isolated from a sample. In some embodiments, the probes and primers are used to identify the Gram-stain status of a bacterium isolated from a sample. In other embodiments, the probes and primers are used to identify the species of a bacterium isolated from a sample. In further embodiments, the probes and primer are used to determine whether a bacterium is resistant to a particular antibiotic.

In some embodiments of the invention, the methods employ real-time PCR. In other embodiments of the invention, the methods employ a DNA microarray.

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were set forth explicitly herein.

### Gram-Stain Status

All bacteria carry multiple copies of a gene encoding the 16S ribosomal RNA within their genome. The compositions and methods of the invention relate to the discovery that there is a region of sequence in the 16S ribosomal RNA that is divergent between Gram-positive and Gram-negative bacteria. In various methods of the invention, a probe that is homologous to the Gram-positive sequence within this divergent region is used to determine whether an unknown bacterium is a Gram-positive bacterium or a Gram-negative bacterium. In other various methods of the invention, a probe that is homologous to the Gram-negative sequence within this divergent region is used to determine whether an unknown bacterium is a Gram-positive bacterium or a Gram-negative bacterium. The compositions and methods of the invention also relate to the discovery that this divergent region is flanked both upstream and downstream by a region of sequence that is highly conserved between Gram-positive and Gram-negative bacteria. In various methods of the invention, PCR primers that are homologous to these conserved flanking regions are used to initiate a PCR reaction to amplify the divergent sequence.

The invention provides compositions and methods comprising at least one pair of primers and at least one corresponding probe. In one embodiment, the pair of primers is TACGGGAGGCAGCAGT (SEQ ID NO: 40) and AGCAGCCGCGGTAATA (SEQ ID NO: 41). The skilled artisan will understand that these primers are exemplary, and that the length of the primers useful in the invention can be increased or decreased, depending on the reaction conditions and the desired hybridization stringency.

In various embodiments, the corresponding probe is a least one probe selected from the group consisting of GAGCAACGCCGCGTGA (SEQ ID NO: 42) and CAGCCATGCCGCGTGT (SEQ ID NO: 43). The skilled artisan will understand that this probe is exemplary, and that the length of the probe useful in the invention can be increased or decreased, depending on the reaction conditions and the desired hybridization stringency.

The invention provides methods of identifying the Gram-stain status of a bacterium present in a biological sample. Generally, the method comprises the steps of obtaining a nucleic acid of a bacterium from a biological sample, generating a nucleic acid amplification product of the nucleic acid and detecting the presence or absence of the amplification product by its ability to hybridize to a probe of the invention.

In one embodiment, the invention comprises a diagnostic test to detect a gram negative bacterium. In one embodiment, primers comprising SEQ ID NO: 40 and SEQ ID NO: 41 and probes comprising SEQ ID NO: 43 are used to detect gram negative bacteria. In one embodiment, real time PCR is used to amplify nucleotide sequences isolated from bacteria. In this embodiment, observing the detection of amplified products using the probe, SEQ ID NO: 43, identifies the bacteria as being gram negative. In another embodiment, the lack of detection of amplified products using the probe, SEQ ID NO: 43, identifies the bacteria as not being gram negative.

In one embodiment, the invention comprises a diagnostic test to detect a gram positive bacterium. In one embodiment, primers comprising SEQ ID NO: 40 and SEQ ID NO: 41 and probes comprising SEQ ID NO: 42 are used to detect gram positive bacteria. In one embodiment, real time PCR is used to amplify nucleotide sequences isolated from bacteria. In this embodiment, observing the detection of amplified products using the probe, SEQ ID NO: 42, identifies the bacteria as being gram positive. In another embodiment, the lack of detection of amplified products using the probe, SEQ ID NO: 42, identifies the bacteria as not being gram positive.

### Sequences

TABLE 1
Nucleic Acid Sequences

| Strain/ | | Gram- | |
|---|---|---|---|
| Desig- | | stain | |
| nator | Bacterium | status | Sequence* |
| 06 | Pseudomonas | Negative | TACGGGAGGCAGCAGTGGGGA |
| | aeruginosa | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTAAGTTAATACCTT |
| | | | GCTGTTTTGACGTTACCAACAG |
| | | | AATAAGCACCGGCTAACTTCGT |
| | | | GCCAGCAGCCGCGGTAATA |
| | | | (SEQ ID NO: 1) |
| SpA | Pseudomonas | Negative | TACGGGAGGCAGCAGTGGGGA |
| | aeruginosa | | ATATTGGACAAGGGCGAAAGC |
| | | | CTGATCCAGCCATGCCGCGTGT |
| | | | GTGAAGAAGGTCTTCGGATTGT |
| | | | AAAGCACTTTAAGTTGGGAGGA |
| | | | AGGGCAGTAAGTTAATACCTTG |
| | | | CTGTTTTGACGTTACCAACAGA |
| | | | ATAAGCACCGGCTAACTTCGTG |
| | | | CCAGCAGCCGCGGTAATA |
| | | | (SEQ ID NO: 2) |
| 08 | Pseudomonas | Negative | TACGGGAGGCAGCAGTGGGGA |
| | aeruginosa | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTAAGTTAATACCTT |
| | | | GCTGTTTTGACGTTACCAACAG |
| | | | AATAAGCACCGGCTAACTTCGT |
| | | | GCCAGCAGCCGCGGTAATA |
| | | | (SEQ ID NO: 3) |
| SpB | Pseudomonas | Negative | TACGGGAGGCAGCAGTGGGGA |
| | aeruginosa | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTAAGTTAATACCTT |
| | | | GCTGTTTTGACGTTACCAACAG |
| | | | AATAAGCACCGGCTAACTTCGT |
| | | | GCCAGCAGCCGCGGTAATA |
| | | | (SEQ ID NO: 4) |
| DoD6 | Pseudomonas | Negative | TACGGGAGGCAGCAGTGGGGA |
| | aeruginosa | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTAAGTTAATACCTT |
| | | | GCTGTTTTGACGTTACCAACAG |
| | | | AATAAGCACCGGCTAACTTCGT |
| | | | GCCAGCAGCCGCGGTAATA |
| | | | (SEQ ID NO: 5) |
| SpS | Pseudomonas | Negative | TACGGGAGGCAGCAGTGGGGA |
| | putida | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTAAGTTAATACCTT |
| | | | GCTGTTTTGACGTTACCGACAG |
| | | | AATAAGCACCGGCTAACTCTGT |
| | | | GCCAGCAGCCGCGGTAATA |
| | | | (SEQ ID NO: 6) |
| SpN | Enterobacter | Negative | TACGGGAGGCAGCAGTGGGGA |
| | aerogenes | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGAGGAGG |
| | | | AAGGCGTTAAGGTTAATAACCT |
| | | | TGGCGATTGACGTTACTCGCAG |
| | | | AAGAAGCACCGGCTAACTCCGT |
| | | | GCCAGCAGCCGCGGTAATA |
| | | | (SEQ ID NO: 7) |
| SpV | Enterobacter | Negative | TACGGGAGGCAGCAGTGGGGA |
| | sakazakii | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGAGGAGG |
| | | | AAGGCATTAAGGTTAATAACCT |
| | | | TAGTGATTGACGTTACTCGCAG |
| | | | AAGAAGCACCGGCTAACTCCGT |
| | | | GCCAGCAGCCGCGGTAATA |
| | | | (SEQ ID NO: 8) |
| DoD2 | Enterobacter | Negative | TACGGGAGGCAGCAGTGGGGA |
| | cloacae | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGTGTTGTGGTTAATAACCG |
| | | | CAGCAATTGACGTTACCCGCAG |
| | | | AAGAAGCACCGGCTAACTCCGT |
| | | | GCCAGCAGCCGCGGTAATA |
| | | | (SEQ ID NO: 9) |
| SpF | Citrobacter | Negative | TACGGGAGGCAGCAGTGGGGA |
| | koseri | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGTGTTGTGGTTAATAACCG |
| | | | CAGCAATTGACGTTACCCGCAG |
| | | | AAGAAGCACCGGCTAACTCCGT |
| | | | GCCAGCAGCCGCGGTAATA |
| | | | (SEQ ID NO: 10) |
| SpL | Citrobacter | Negative | TACGGGAGGCAGCAGTGGGGA |
| | freundii | | ATATTGCACAATGGGCACAAGA |
| | | | CTGATGCAGCCATGCCGCGTGT |
| | | | ATGAAGAAGGCCTTCGGGTTGT |
| | | | AAAGTACTTTCAGCGAGGAGG |
| | | | AAGGCGTTGTGGTTAATAACCG |
| | | | CAGCGATTGACGTTACTCGCAG |
| | | | AAGAAGCACCGGCTAACTCCGT |
| | | | GCCAGCAGCCGCGGTAATA |
| | | | (SEQ ID NO: 11) |
| SpM | Citrobacter | Negative | TACGGGAGGCAGCAGTGGGGA |
| | freundii | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGAGGAGG |
| | | | AAGGCGTTGTGGTTAATAACCG |
| | | | CAGCGATTGACGTTACTCGCAG |
| | | | AAGAAGCACCGGCTAACTCCGT |
| | | | GCCAGCAGCCGCGGTAATA |
| | | | (SEQ ID NO: 12) |
| SpX | Klebsiella | Negative | TACGGGAGGCAGCAGTGGGGA |
| | terrigena | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGTGTTGAGGTTAATAACCT |
| | | | CAGCAATTGACGTTACCCGCAG |
| | | | AAGAAGCACCGGCTAACTCCGT |
| | | | GCCAGCAGCCGCGGTAATA |
| | | | (SEQ ID NO: 13) |
| SpE | Klebsiella | Negative | TACGGGAGGCAGCAGTGGGGA |
| | pneumoniae | | ATATTGCACAATGGGCGCAAGC |
| | | | CTGATGCAGCCATGCCGCGTGT |
| | | | GTGAAGAAGGCCTTCGGGTTGT |
| | | | AAAGCACTTTCAGCGGGGAGG |
| | | | AAGGCGGTGAGGTTAATAACCT |
| | | | CACCGATTGACGTTACCCGCAG |
| | | | AAGAAGCACCGGCTAACTCCGT |
| | | | GCCAG CAGCCGCGGTAATA |
| | | | (SEQ ID NO: 14) |
| SpW | Citrobacter | Negative | TACGGGAGGCAGCAGTGGGGA |
| | youngae | | ATATTGCACAATGGGCGCAAGC |
| | | | CTGATGCAGCCATGCCGCGTGT |
| | | | GTGAAGAAGGCCTTCGGGTTGT |
| | | | AAAGCACTTTCAGCGGGGAGG |
| | | | AAGGCGGTAAGGTTAATAACCT |
| | | | CGTCGATTGACGTTACCCGCAG |
| | | | AAGAAGCACCGGCTAACTCCGT |
| | | | GCCAGCAGCCGCGGTAATA |
| | | | (SEQ ID NO: 15) |
| DoD5 | Klebsiella | Negative | TACGGGAGGCAGCAGTGGGGA |
| | pneumoniae | | ATATTGCACAATGGGCGCAAGC |
| | | | CTGATGCAGCCATGCCGCGTGT |
| | | | GTGAAGAAGGCCTTCGGGTTGT |
| | | | AAAGCACTTTCAGCGGGGAGG |
| | | | AAGGCGTTGAGGTTAATAACCT |
| | | | CGGCGATTGACGTTACCCGCAG |
| | | | AAGAAGCACCGGCTAACTCCGT |
| | | | GCCAGCAGCCGCGGTAATA |
| | | | (SEQ ID NO: 16) |
| SpP | Proteus | Negative | TACGGGAGGCAGCAGTGGGGA |
| | vulgaris | | ATATTGCACAATGGGCGCAAGC |
| | | | CTGATGCAGCCATGCCGCGTGT |
| | | | ATGAAGAAGGCCCTAGGGTTGT |
| | | | AAAGTACTTTCAGTCGGGAGGA |
| | | | AGGCGTTGATGTTAATACCATC |
| | | | AACGATTGACGTTACCGACAGA |
| | | | AGAAGCACCGGCTAACTCCGTG |
| | | | CCAGCAGCCGCGGTAATA |
| | | | (SEQ ID NO: 17) |
| SpQ | Providencia | Negative | TACGGGAGGCAGCAGTGGGGA |
| | stuartii | | ATATTGCACAATGGGCGCAAGC |
| | | | CTGATGCAGCCATGCCGCGTGT |
| | | | ATGAAGAAGGCCCTAGGGTTGT |
| | | | AAAGTACTTTCAGTCGGGAGGA |
| | | | AGGCGTTGATGTTAATACCATC |
| | | | AACGATTGACGTTACCGACAGA |
| | | | AGAAGCACCGGCTAACTCCGTG |
| | | | CCAGCAGCCGCGGTAATA |
| | | | (SEQ ID NO: 18) |
| SpJ | Morganella | Negative | TACGGGAGGCAGCAGTGGGGA |
| | morganii | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGTCGGGAGGA |
| | | | AGGTGTCAAGGTTAATAACCTT |
| | | | GGCAATTGACGTTACCGACAGA |
| | | | AGAAGCACCGGCTAACTCCGTG |
| | | | CCAGCAGCCGCGGTAATA |
| | | | (SEQ ID NO: 19) |
| 07 | Escherichia | Negative | TACGGGAGGCAGCAGTGGGGA |
| | coli | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGGAGTAAAGTTAATACCTT |
| | | | TGCTCATTGACGTTACCCGCAG |
| | | | AAGAAGCACCGGCTAACTCCGT |
| | | | GCCAGCAGCCGCGGTAATA |
| | | | (SEQ ID NO: 20) |
| SpG | Escherichia | Negative | TACGGGAGGCAGCAGTGGGGA |
| | coli | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGGAGTAAAGTTAATACCTT |
| | | | TGCTCATTGACGTTACCCGCAG |
| | | | AAGAAGCACCGGCTAACTCCGT |
| | | | GCCAGCAGCCGCGGTAATA |
| | | | (SEQ ID NO: 21) |
| SpI | Shigella | Negative | TACGGGAGGCAGCAGTGGGGA |
| | flexneri | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGGAGTAAAGTTAATACCTT |
| | | | TGCTCATTGACGTTACCCGCAG |
| | | | AAGAAGCACCGGCTAACTCCGT |
| | | | GCCAGCAGCCGCGGTAATA |
| | | | (SEQ ID NO: 22) |
| SpO | Klebsiella | Negative | TACGGGAGGCAGCAGTGGGGA |
| | oxytoca | | ATATTGCACAATGGGCGCAAGC |
| | | | CTGATGCAGCCATGCCGCGTGT |
| | | | ATGAAGAAGGCCTTCGGGTTGT |
| | | | AAAGTACTTTCAGCGGGGAGG |
| | | | AAGGGAGTGAGGTTAATAACCT |
| | | | TATTCATTGACGTTACCCGCAG |
| | | | AAGAAGCACCGGCTAACTCCGT |
| | | | GCCAGCAGCCGCGGTAATA |
| | | | (SEQ ID NO: 23) |
| SpD | Proteus | Negative | TACGGGAGGCAGCAGTGGGGA |
| | mirabilis | | ATATTGCACAATGGGCGCAAGC |
| | | | CTGATGCAGCCATGCCGCGTGT |
| | | | ATGAAGAAGGCCTTAGGGTTGT |
| | | | AAAGTACTTTCAGCGGGGAGG |
| | | | AAGGTGATAAGGTTAATACCCT |
| | | | TATCAATTGACGTTACCCGCAG |
| | | | AAGAAGCACCGGCTAACTCCGT |
| | | | GCCAGCAGCCGCGGTAATA |
| | | | (SEQ ID NO: 24) |
| DoD7 | Proteus | Negative | TACGGGAGGCAGCAGTGGGGA |
| | mirabilis | | ATATTGCACAATGGGCGCAAGC |
| | | | CTGATGCAGCCATGCCGCGTGT |
| | | | ATGAAGAAGGCCTTAGGGTTGT |
| | | | AAAGTACTTTCAGCGGGGAGG |
| | | | AAGGTGATAAGGTTAATACCCT |
| | | | TATCAATTGACGTTACCCGCAG |
| | | | AAGAAGCACCGGCTAACTCCGT |
| | | | GCCAGCAGCCGCGGTAATA |
| | | | (SEQ ID NO: 25) |
| SpT | Serratia | Negative | TACGGGAGGCAGCAGTGGGGA |
| | marcescens | | ATATTGCACAATGGGCGCAAGC |
| | | | CTGATGCAGCCATGCCGCGTGT |
| | | | GTGAAGAAGGCCTTCGGGTTGT |
| | | | AAAGCACTTTCAGCGAGGAGG |
| | | | AAGGTGGTGAACTTAATACGTT |
| | | | CATCAATTGACGTTACTCGCAG |
| | | | AAGAAGCACCGGCTAACTCCGT |
| | | | GCCAGCAGCCGCGGTAATA |
| | | | (SEQ ID NO: 26) |
| DoD1 | Acinetobacter | Negative | TACGGGAGGCAGCAGTGGGGA |
| | baumannii | | ATATTGGACAATGGGGGGAAC |
| | | | CCTGATCCAGCCATGCCGCGTG |
| | | | TGTGAAGAAGGCCTTATGGTTG |
| | | | TAAAGCACTTTAAGCGAGGAG |
| | | | GAGGCTACTTTAGTTAATACCT |
| | | | AGAGATAGTGGACGTTACTCGC |
| | | | AGAATAAGCACCGGCTAACTCT |
| | | | GTGCCAGCAGCCGCGGTAATA |
| | | | (SEQ ID NO: 27) |
| SpC | Enterococcus | Positive | TACGGGAGGCAGCAGTAGGGA |
| | faecalis | | ATCTTCGGCAATGGACGAAAGT |
| | | | CTGACCGAGCAACGCCGCGTGA |
| | | | GTGAAGAAGGTTTTCGGATCGT |
| | | | AAAACTCTGTTGTTAGAGAAGA |
| | | | ACAAGGACGTTAGTAACTGAAC |
| | | | GTCCCCTGACGGTATCTAACCA |
| | | | GAAAGCCACGGCTAACTACGTG |
| | | | CCAGCAGCCGCGGTAATA |
| | | | (SEQ ID NO: 28) |
| DoD3 | Enterococcus | Positive | TACGGGAGGCAGCAGTAGGGA |
| | faecalis | | ATCTTCGGCAATGGACGAAAGT |
| | | | CTGACCGAGCAACGCCGCGTGA |
| | | | GTGAAGAAGGTTTTCGGATCGT |
| | | | AAAACTCTGTTGTTAGAGAAGA |
| | | | ACAAGGACGTTAGTAACTGAAC |
| | | | GTCCCCTGACGGTATCTAACCA |
| | | | GAAAGCCACGGCTAACTACGTG |
| | | | CCAGCAGCCGCGGTAATA |
| | | | (SEQ ID NO: 29) |
| DoD4 | Enterococcus | Positive | TACGGGAGGCAGCAGTAGGGA |
| | faecium | | ATCTTCGGCAATGGACGAAAGT |
| | | | CTGACCGAGCAACGCCGCGTGA |
| | | | GTGAAGAAGGTTTTCGGATCGT |
| | | | AAAACTCTGTTGTTAGAGAAGA |
| | | | ACAAGGATGAGAGTAACTGTTC |
| | | | ATCCCTTGACGGTATCTAACCA |
| | | | GAAAGCCACGGCTAACTACGTG |
| | | | CCAGCAGCCGCGGTAATA |
| | | | (SEQ ID NO: 30) |
| DoD11 | Streptococcus | Positive | TACGGGAGGCAGCAGTAGGGA |
| | pyogenes | | ATCTTCGGCAATGGGGGCAACC |
| | | | CTGACCGAGCAACGCCGCGTGA |
| | | | GTGAAGAAGGTTTTCGGATCGT |
| | | | AAAGCTCTGTTGTTAGAGAAGA |
| | | | ATGATGGTGGGAGTGGAAAAT |
| | | | CCACCAAGTGACGGTAACTAAC |
| | | | CAGAAAGGGACGGCTAACTAC |
| | | | GTGCCAGCAGCCGCGGTAATA |
| | | | (SEQ ID NO: 31) |
| Strep B | Streptococcus | Positive | TACGGGAGGCAGCAGTAGGGA |
| | agalactiae | | ATCTTCGGCAATGGACGGAAGT |
| | | | CTGACCGAGCAACGCCGCGTGA |
| | | | GTGAAGAAGGTTTTCGGATCGT |
| | | | AAAGCTCTGTTGTTAGAGAAGA |
| | | | ACGTTGGTAGGAGTGGAAAATC |
| | | | TACCAAGTGACGGTAACTAACC |
| | | | AGAAAGGGACGGCTAACTACG |
| | | | TGCCAGCAGCCGCGGTAATA |
| | | | (SEQ ID NO: 32) |
| DoD10 | Streptococcus | Positive | TACGGGAGGCAGCAGTAGGGA |
| | pneumoniae | | ATCTTCGGCAATGGACGGAAGT |
| | | | CTGACCGAGCAACGCCGCGTGA |
| | | | GTGAAGAAGGTTTTCGGATCGT |
| | | | AAAGCTCTGTTGTAAGAGAAGA |
| | | | ACGAGTGTGAGAGTGGAAAGT |
| | | | TCACACTGTGACGGTATCTTAC |
| | | | CAGAAAGGGACGGCTAACTAC |
| | | | GTGCCAGCAGCCGCGGTAATA |
| | | | (SEQ ID NO: 33) |
| DoD8 | Staphylococcus | Positive | TACGGGAGGCAGCAGTAGGGA |
| | aureus | | ATCTTCCGCAATGGGCGAAAGC |
| | | | CTGACGGAGCAACGCCGCGTG |
| | | | AGTGATGAAGGTCTTCGGATCG |
| | | | TAAAACTCTGTTATTAGGGAAG |
| | | | AACATATGTGTAAGTAACTGTG |
| | | | CACATCTTGACGGTACCTAATC |
| | | | AGAAAGCCACGGCTAACTACGT |
| | | | GCCAGCAGCCGCGGTAATA |
| | | | (SEQ ID NO: 34) |
| DoD9 | Staphylococcus | Positive | TACGGGAGGCAGCAGTAGGGA |
| | aureus | | ATCTTCCGCAATGGGCGAAAGC |
| | (MRSA) | | CTGACGGAGCAACGCCGCGTG |
| | | | AGTGATGAAGGTCTTCGGATCG |
| | | | TAAAACTCTGTTATTAGGGAAG |
| | | | AACATATGTGTAAGTAACTGTG |
| | | | CACATCTTGACGGTACCTAATC |
| | | | AGAAAGCCACGGCTAACTACGT |
| | | | GCCAGCAGCCGCGGTAATA |
| | | | (SEQ ID NO: 35) |
| SpK | Staphylococcus | Positive | TACGGGAGGCAGCAGTAGGGA |
| | coagulase + | | ATCTACCACAATGGGCGAAAGC |
| | | | CTGACGGAGCAACGCCGCGTG |
| | | | AGTGATGAAGGTCTTCGGATCG |
| | | | TAAAACTCTGTTATTAGGGAAG |
| | | | AACATATGTGTAAGTAACTGTG |
| | | | CACATCTTGACGGTACCTAATC |
| | | | AGAAAGCCACGGCTAACTACGT |
| | | | GCCAGCAGCCGCGGTAATA |
| | | | (SEQ ID NO: 36) |
| SpH | Staphylococcus | Positive | TACGGGAGGCAGCAGTAGGGA |
| | epidermidis | | ATCTTCCGCAATGGGCGAAAGC |
| | | | CTGACGGAGCAACGCCGCGTG |
| | | | AGTGATGAAGGTCTTCGGATCG |
| | | | TAAAACTCTGTTATTAGGGAAG |
| | | | AACAAATGTGTAAGTAACTATG |
| | | | CACGTCTTGACGGTACCTAATC |
| | | | AGAAAGCCACGGCTAACTACGT |
| | | | GCCAGCAGCCGCGGTAATA |
| | | | (SEQ ID NO: 37) |
| SpU | Staphylococcus | Positive | TACGGGAGGCAGCAGTAGGGA |
| | epidermidis | | ATCTTCCGCAATGGGCGAAAGC |
| | | | CTGACGGAGCAACGCCGCGTG |
| | | | AGTGATGAAGGTCTTCGGATCG |
| | | | TAAAACTCTGTTATTAGGGAAG |
| | | | AACAAATGTGTAAGTAACTATG |
| | | | CACGTCTTGACGGTACCTAATC |
| | | | AGAAAGCCACGGCTAACTACGT |
| | | | GCCAGCAGCCGCGGTAATA |
| | | | (SEQ ID NO: 38) |
| SpR | Pseudomonas | Negative | TACGGGAGGCAGCAGTGGGGA |
| | fluorescens | | ATATTGGACAATGGGCGAAAG |
| | | | CCTGATCCAGCCATGCCGCGTG |
| | | | TGTGAAGAAGGTCTTCGGATTG |
| | | | TAAAGCACTTTAAGTTGGGAGG |
| | | | AAGGGCAGTTACCTAATACGTG |
| | | | ATTGTTTTGACGTTACCGACAG |
| | | | AATAAGCACCGGCTAACTCTGT |
| | | | GCCAGCAGCCGCGGTAATA |
| | | | (SEQ ID NO: 39) |
| SP1 | Enterobacter | | GGGATGACGTCAAGTCATCATG |
| | cloacae | | GCCCTTACGACCAGGGCTACAC |
| | | | ACGAGAAACAATGGCG |
| | | | CATACAAAGAGAAGCGACCTC |
| | | | GCGAGAGCAAGCGGACCTCAT |
| | | | AAAGTGCGTCGTAGTCCG |
| | | | GATTGGAGTCTGCAACTCGACT |
| | | | CCATGAAGTCGGAATCGCTAGT |
| | | | AATCG (SEQ ID NO: 57) |
| SP2 | Pseudomonas | | GAAGTCGGAATCGCTAGTAATC |
| | aeruginosa | | GTGAATCAGAATGTCACGGTGA |
| | | | ATACGTTCCCGGGCCT |
| | | | TGTACACACCGCCCGTCACACC |
| | | | ATGGGAGTGGGTTGCTCCAGAA |
| | | | GTAGCTAGTCTAACCG |
| | | | CAAGGGGGACGGTTACCACGG |
| | | | AGTGATTCATGACTGGGGTGAA |
| | | | GTCGTAACAAGGTATCC |
| | | | GTA (SEQ ID NO: 58) |
| SP3 | Acinetobacter | | GAAGTCGGAATCGCTAGTAATC |
| | baumannii | | GCGGATCAGAATGCCGCGGTG |
| | | | AATACGTTCCCGGGCCT |
| | | | TGTACACACCGCCCGTCACACC |
| | | | ATGGGAGTTTGTTGCACCAGAA |
| | | | GTAGCTAGCCTAACTG |
| | | | CAAAGAGGGCGGTTACCACGG |
| | | | TGTGGCCGATGACTGGGGTGAA |
| | | | GTCGTAACAAGGTATCC |
| | | | GTA (SEQ ID NO: 59) |
| SP5 | Citrobacter | | GAGTTTGATCATGGCTCAGATT |
| | koseri | | GAACGCTGGCGGCAGGCCTAA |
| | | | CACATGCAAGTCGAACG |
| | | | GTAACAGGAAGCAGCTTGCTGC |
| | | | TTTGCTGACGAGTGGCGGACGG |
| | | | GTGAGTAATGTCTGGG |
| | | | AAACTGCCTGATGGAGGGGGA |
| | | | TAACTACTGGAAACGGTAGCTA |
| | | | ATACCGCATAACGTCGC |
| | | | AAGACCAAAGAGGGGGACCTT |
| | | | CGGGCCTCTTGCCATCAGATGT |
| | | | GCCCAGATGGGATTAGCTAGTT |
| | | | GGTGGGGTAACGGCTCACCAA |
| | | | GGCGACGATCCCTAGCTGGTCT |
| | | | GAGAGGATGACCGCCACACTG |
| | | | GAACTGAGACACGGTCCAGACT |
| | | | CCTACGGGAGGCAGCAGTGGG |
| | | | G (SEQ ID NO: 60) |
| SP6 | Klebsiella | | GAGTTTGATCATGGCTCAGATT |
| | pneumoniae | | GAACGCTGGCGGCAGGCCTAA |
| | | | CACATGCAAGTCGAGCGGTAGC |
| | | | ACAGAGAGCTTGCTCTCGGGTG |
| | | | ACGAGCGGCGGACGGGTGAGT |
| | | | AATGTCTGGGAAACTGCCTGAT |
| | | | GGAGGGGGATAACTACTGGAA |
| | | | ACGGTAGCTAATACCGCATAAT |
| | | | GTCGCAAGACCAAAGTGGGGG |
| | | | ACCTTCGGGCCTCATGCCATCA |
| | | | GATGTGCCCAGATGGGATTAGC |
| | | | TAGTAGGTGGGGTAACGGCTCA |
| | | | CCTAGGCGACGATCCCTAGCTG |
| | | | GTCTGAGAGGATGACCAGCCAC |
| | | | ACTGGAACTGAGACACGGTCCA |
| | | | GACTCCTACGGGAGGCAGCAGT |
| | | | GGGG (SEQ ID NO: 61) |
| SP7 | Proteus | | GGAAGACGTCAAGTCATCATGG |
| | mirabilis | | CCCTTACGAGTAGGGCTACACA |
| | | | CGTGCTACAATGGCAGATACAA |
| | | | AGAGAAGCGACCTCGCGAGAG |
| | | | CAAGCGGAACTCATAAAGTCTG |
| | | | TCGTAGTCCGGATTGGAGTCTG |
| | | | CAACTCGACTCCATGAAGTCGG |
| | | | AATCGCTAGTAATCG |
| | | | (SEQ ID NO: 62) |
| SP8 | Morganella | | GAGTTTGATCATGGCTCAGATT |
| | moranii | | GATCGCTGGCGGCAGGCCTAAC |
| | | | ACATGCAAGTCGGGCGGTAAC |
| | | | AGGGAGAAGCTTGCTTCTCTGC |
| | | | TGACGAGCGGCGGACGGGTGA |
| | | | GTAATGTATGGGGATCTGCCTG |
| | | | ATGGCGGGGGTTAACTACTGGA |
| | | | AACGGTAGCTAATACCGCATAA |
| | | | TGTCTACGGACCAAAGCGGGG |
| | | | GACCTCCGGGCCTCGCGCCATC |
| | | | AGATGAACCCATATGGGATTAG |
| | | | CTAGTAGGTAAGGTAACGGCTT |
| | | | ACCTAGGCGACGATCCCTAGCT |
| | | | GGTCTGAGAGGATGATCAGCCA |
| | | | CACTGGGACTGAGACACGGCCC |
| | | | AGACTCCTACGGGAGGCAGCA |
| | | | GTGGGG (SEQ ID NO: 63) |
| SP8 | Morganella | | GGATGACGTCAAGTCATCATGG |
| | moranii | | CCCTTACGAGTAGGGCTACACA |
| | | | CGTGCTACAATGGCGT |
| | | | ATACAAAGGGAAGCGACCCCG |
| | | | CGAGGGCAAGCGGAACTCATA |
| | | | AAGTACGTCGTAGTCCGG |
| | | | ATTGGAGTCTGCAACTCGACTC |
| | | | CATGAAGTCGGAATCGCTAGTA |
| | | | ATCG (SEQ ID NO: 113) |
| SP9 | Enterococcus | | GGATGAAGACAAATCATCATGC |
| | faecalis | | CCCTTATGACCTGGGCTACACA |
| | | | CGTGCTACAATGGGAAGTACAA |
| | | | CGAGTCGCTAGACCGCGAGGTC |
| | | | ATGCAAATCTCTTAAAGCTTCT |
| | | | CTCAGTTCGGATTGCAGGCTGC |
| | | | AACTCGCCTGCATGAAGCCGGA |
| | | | ATCGCTAGTAATCG |
| | | | (SEQ ID NO: 64) |
| SP10 | Enterococcus | | GGGATGACGTCAAATCATCATG |
| | faecium | | CCCCTTATGACCTGGGCTACAC |
| | | | ACGTGCTACAATGGGAAGTACA |
| | | | ACGAGTTGCGAAGTCGCGAGG |
| | | | CTAAGCTAATCTCTTAAAGCTT |
| | | | CTCTCAGTTCGGATTGCAGGCT |
| | | | GCAACTCGCCTGCATGAAGCCG |
| | | | GAATCGCTAGTAATCG |
| | | | (SEQ ID NO: 65) |
| SP11 | Streptococcus | | GAAGTCGGAATCGCTAGTAATC |
| | pneumoniae | | GCGGATCAGCACGCCGCGGTG |
| | | | AATACGTTCCCGGGCCTTGTAC |
| | | | ACACCGCCCGTCACACCACGAG |
| | | | AGTTTGTAACACCCGAAGTCGG |
| | | | TGAGGTAACCGTAAGGAGCCA |
| | | | GCCGCCTAAGGTGGGATAGATG |
| | | | ATTGGGGTGAAGTCGTAACAAG |
| | | | GTATCCGTA |
| | | | (SEQ ID NO: 66) |
| SP12 | Streptococcus | | GAAGTCGGAATCGCTAGTAATC |
| | pyogenes | | GCGGATCAGCACGCCGCGGTG |
| | | | AATACGTTCCCGGGCCTTGTAC |
| | | | ACACCGCCCGTCACACCACGAG |
| | | | AGTTTGTAACACCCGAAGTCGG |
| | | | TGAGGTAACCTATTAGGAGCCA |
| | | | GCCGCCTAAGGTGGGATAGATG |
| | | | ATTGGGGTGAAGTCGTAACAAG |
| | | | GTATCCGTA |
| | | | (SEQ ID NO: 67) |
| SP13 | Staphylococcus | | GAGTTTGATCATGGCTCAGGAT |
| | aureus | | GAACGCTGGCGGCGTGCCTAAT |
| | | | ACATGCAAGTCGAGCGAACGG |
| | | | ACGAGAAGCTTGCTTCTCTGAT |
| | | | GTTAGCGGCGGACGGGTGAGT |
| | | | AACACGTGGATAACCTACCTAT |
| | | | AAGACTGGGATAACTTCGGGA |
| | | | AACCGGAGCTAATACCGGATA |
| | | | ATATTTTGAACCGCATGGTTCA |
| | | | AAAGTGAAAGACGGTCTTGCTG |
| | | | TCACTTATAGATGGATCCGCGC |
| | | | TGCATTAGCTAGTTGGTAAGGT |
| | | | AACGGCTTACCAAGGCAACGAT |
| | | | GCATAGCCGACCTGAGAGGGT |
| | | | GATCGGCCACACTGGAACTGAG |
| | | | ACACGGTCCAGACTCCTACGGG |
| | | | AGGCAGCAGTAGGG |
| | | | (SEQ ID NO: 68) |
| SP14 | Staphylococcus | | GAGTTTGATCATGGCTCAGGAT |
| | epidermidis | | GAACGCTGGCGGCGTGCCTAAT |
| | | | ACATGCAAGTCGAGCGAACAG |
| | | | ACGAGGAGCTTGCTCCTCTGAC |
| | | | GTTAGCGGCGGACGGGTGAGT |
| | | | AACACGTGGATAACCTACCTAT |
| | | | AAGACTGGGATAACTTCGGGA |
| | | | AACCGGAGCTAATACCGGATA |
| | | | ATATATTGAACCGCATGGTTCA |
| | | | ATAGTGAAAGACGGTTTTGCTG |
| | | | TCACTTATAGATGGATCCGCGC |
| | | | CGCATTAGCTAGTTGGTAAGGT |
| | | | AACGGCTTACCAAGGCAACGAT |
| | | | GCGTAGCCGACCTGAGAGGGT |
| | | | GATCGGCCACACTGGAACTGAG |
| | | | ACACGGTCCAGACTCCTACGGG |
| | | | AGGCAGCAGTAGGGG |
| | | | (SEQ ID NO: 69) |
| SP15 | Pseudomonas | | GAGTTTGATCTTGGCTCAGATT |
| | putida | | GAACGCTGGCGGCAGGCCTAA |
| | | | CACATGCAAGTCGAGCGGATG |
| | | | ACGGGAGCTTGCTCCTTGATTC |
| | | | AGCGGCGGACGGGTGAGTAAT |
| | | | GCCTAGGAATCTGCCTGGTAGT |
| | | | GGGGGACAACGTTTCGAAAGG |
| | | | AACGCTAATACCGCATACGTCC |
| | | | TACGGGAGAAAGCAGGGGACC |
| | | | TTCGGGCCTTGCGCTATCAGAT |
| | | | GAGCCTAGGTCGGATTAGCTAG |
| | | | TTGGTGGGGTAATGGCTCACCA |
| | | | AGGCGACGATCCGTAACTGGTC |
| | | | TGAGAGGATGATCAGTCACACT |
| | | | GGAACTGAGACACGGTCCAGA |
| | | | CTCCTACGGGAGGCAGCAGTGG |
| | | | GG (SEQ ID NO: 70) |
| SP16 | Citrobacter | | GAGTTTGATCTTGGCTCAGATT |
| | freundii | | GAACGCTGGCGGCAGGCCTAA |
| | | | CACATGCAAGTCGAACGGTAGC |
| | | | ACAGAGGAGCTTGCTCCTTGGG |
| | | | TGACGAGTGGCGGACGGGTGA |
| | | | GTAATGTCTGGGAAACTGCCCG |
| | | | ATGGAGGGGGATAACTACTGG |
| | | | AAACGGTAGCTAATACCGCATA |
| | | | ACGTCGCAAGACCAAAGAGGG |
| | | | GGACCTTCGGGCCTCTTGCCAT |
| | | | CGGATGTGCCCAGATGGGATTA |
| | | | GCTAGTAGGTGGGGTAACGGCT |
| | | | CACCTAGGCGACGATCCCTAGC |
| | | | TGGTCTGAGAGGATGACCAGCC |
| | | | ACACTGGAACTGAGACACGGTC |
| | | | CAGACTCCTACGGGAGGCAGC |
| | | | AGTGGGG (SEQ ID NO: 71) |
| SP17 | Enterobacter | | GAGTTTGATCTTGGCTCAGATT |
| | sakazakii | | GAACGCTGGCGGCAGGCCTAA |
| | | | CACATGCAAGTCGAACGGTAGC |
| | | | ACAGAGGAGGCTTGCTCCTTGG |
| | | | GTGACGAGTGGCGGACGGGTG |
| | | | AGTAATGTCTGGGAAACTGCCC |
| | | | GATGGAGGGGGATAACTACTG |
| | | | GAAACGGTAGCTAATACCGCAT |
| | | | AACGTCGCAAGACCAAAGAGG |
| | | | GGGACCTTCGGGCCTCTTGCCA |
| | | | TCGGATGTGCCCAGATGGGATT |
| | | | AGCTAGTAGGTGGGGTAACGG |
| | | | CTCACCTAGGCGACGATCCCTA |
| | | | GCTGGTCTGAGAGGATGACCAG |
| | | | CCACACTGGAACTGAGACACG |
| | | | GTCCAGACTCCTACGGGAGGCA |
| | | | GCAGTGGGG |
| | | | (SEQ ID NO: 72) |
| SP18 | Klebsiella | | GAGTTTGATCTTGGCTCAGATT |
| | terrigena | | GAACGCTGGCGGCAGGCCTAA |
| | | | CACATGCAAGTCGAACGGTAAC |
| | | | AGGAAGCAGCTTGCTGCTTCGC |
| | | | TGACGAGTGGCGGACGGGTGA |
| | | | GTAATGTCTGGGAAACTGCCTG |
| | | | ATGGAGGGGGATAACTACTGG |
| | | | AAACGGTAGCTAATACCGCATA |
| | | | ACGTCGCAAGACCAAAGAGGG |
| | | | GGACCTTCGGGCCTCTTGCCAT |
| | | | CGGATGTGCCCAGATGGGATTA |
| | | | GCTAGTAGGTGGGGTAACGGCT |
| | | | CACCTAGGCGACGATCCCTAGC |
| | | | TGGTCTGAGAGGATGACCAGCC |
| | | | ACACTGGAACTGAGACACGGTC |
| | | | CAGACTCCTACGGGAGGCAGC |
| | | | AGTGGGG (SEQ ID NO: 73) |
| SP19 | Klebsiella | | GAGTTTGATCTTGGCTCAGATT |
| | oxytoca | | GAACGCTGGCGGCAGGCCTAA |
| | | | CACATGCAAGTCGAACGGTAGC |
| | | | ACAGAGAGCTTGCTCTCGGGTG |
| | | | ACGAGTGGCGGACGGGTGAGT |
| | | | AATGTCTGGGAAACTGCCCGAT |
| | | | GGAGGGGGATAACTACTGGAA |
| | | | ACGGTAGCTAATACCGCATAAT |
| | | | GTCGCAAGACCAAAGAGGGGG |
| | | | ACCTTCGGGCCTCTTGCCATCG |
| | | | GATGTGCCCAGATGGGATTAGC |
| | | | TTGTAGGTGAGGTAACGGCTCA |
| | | | CCTAGGCGACGATCCCTAGCTG |
| | | | GTCTGAGAGGATGACCAGCCAC |
| | | | ACTGGAACTGAGACACGGTCCA |
| | | | GACTCCTACGGGAGGCAGCAGT |
| | | | GGGG (SEQ ID NO: 74) |
| SP21 | Serratia | | GAGTTTGATCTTGGCTCAGATT |
| | marcescens | | GAACGCTGGCGGCAGGCTTAAC |
| | | | ACATGCAAGTCGAGCGGTAGC |
| | | | ACAAGGGAGCTTGCTCCCTGGG |
| | | | TGACGAGCGGCGGACGGGTGA |
| | | | GTAATGTCTGGGAAACTGCCTG |
| | | | ATGGAGGGGGATAACTACTGG |
| | | | AAACGGTAGCTAATACCGCATA |
| | | | ACGTCGCAAGACCAAAGAGGG |
| | | | GGACCTTCGGGCCTCTTGCCAT |
| | | | CAGATGTGCCCAGATGGGATTA |
| | | | GCTAGTAGGTGGGGTAATGGCT |
| | | | CACCTAGGCGACGATCCCTAGC |
| | | | TGGTCTGAGAGGATGACCAGCC |
| | | | ACACTGGAACTGAGACACGGTC |
| | | | CAGACTCCTACGGGAGGCAGC |
| | | | AGTGGGG (SEQ ID NO: 75) |
| SP22 | Staphylococcus | | GAGTTTGATCTTGGCTCAGGAT |
| | lugdunensis | | GAACGCTGGCGGCGTGCCTAAT |
| | | | ACATGCAAGTCGAGCGAACAG |
| | | | ATAAGGAGCTTGCTCCTTTGAC |
| | | | GTTAGCGGCGGACGGGTGAGT |
| | | | AACACGTGGGTAACCTACCTAT |
| | | | AAGACTGGGACAACTTCGGGA |
| | | | AACCGGAGCTAATACCGGATA |
| | | | ATATGTTGAACCGCATGGTTCA |
| | | | ATAGTGAAAGATGGTTTTGCTA |
| | | | TCACTTATAGATGGACCCGCGC |
| | | | CGTATTAGCTAGTTGGTGAGGT |
| | | | AACGGCTCACCAAGGCAACGA |
| | | | TACGTAGCCGACCTGAGAGGGT |
| | | | GATCGGCCACACTGGAACTGAG |
| | | | ACACGGTCCAGACTCCTACGGG |
| | | | AGGCAGCAGTAGGG |
| | | | (SEQ ID NO: 76) |
| SP23 | Providecia | | GAGTTTGATCTTGGCTCAGATT |
| | stuartii | | GAACGCTGGCGGCAGGCCTAA |
| | | | CACATGCAAGTCGAGCGGTAAC |
| | | | AGGGGAAGCTTGCTTCTCGCTG |
| | | | ACGAGCGGCGGACGGGTGAGT |
| | | | AATGTATGGGGATCTGCCCGAT |
| | | | AGAGGGGGATAACTACTGGAA |
| | | | ACGGTGGCTAATACCGCATAAT |
| | | | CTCTTAGGAGCAAAGCAGGGG |
| | | | ACCTTCGGGCCTTGCGCTGTCG |
| | | | GATGAACCCATATGGGATTAGC |
| | | | TAGTAGGTAAGGTAATGGCTTA |
| | | | CCTAGGCGACGATCCCTAGCTG |
| | | | GTCTGAGAGGATGATCAGCCAC |
| | | | ACTGGGACTGAGACACGGCCC |
| | | | AGACTCCTACGGGAGGCAGCA |
| | | | GTGGGG (SEQ ID NO: 77) |
| SP24 | Streptococcus | | GAGTTTGATCTTGGCTCAGGAC |
| | agalactiae | | GAACGCTGGCGGCGTGCCTAAT |
| | | | ACATGCAAGTAGAACGCTGAG |
| | | | GTTTGGTGTTTACACTAGACTG |
| | | | ATGAGTTGCGAACGGGTGAGTA |
| | | | ACGCGTAGGTAACCTGCCTCAT |
| | | | AGCGGGGGATAACTATTGGAA |
| | | | ACGATAGCTAATACCGCATAAG |
| | | | AGTAATTAACACATGTTAGTTA |
| | | | TTTAAAAGGAGCAATTGCTTCA |
| | | | CTGTGAGATGGACCTGCGTTGT |
| | | | ATTAGCTAGTTGGTGAGGTAAA |
| | | | GGCTCACCAAGGCGACGATAC |
| | | | ATAGCCGACCTGAGAGGGTGAT |
| | | | CGGCCACACTGGGACTGAGAC |
| | | | ACGGCCCAGACTCCTACGGGAG |
| | | | GCAGCAGTAGGG |
| | | | (SEQ ID NO: 78) |
| LGA251 | Staphylococcus | Positive | GATAAGCATTGGAAATTAGATT |
| | aureus | | GGAGACCAGACGTAATAGTAC |
| | (MRSA) | | CTGGTTTGAAAAATGGACAGAA |
| | | | AATTAATATAGAAACATTAAAA |
| | | | TCAGAGCGAGGCAAAATAAAA |
| | | | GATAGAAATGGTATAGAATTAG |
| | | | CT (SEQ ID NO: 78) |

*underlined nucleotides represent exemplary flanking primer sequences and exemplary internal probe sequences

### Methicillin Resistance

One of the most pressing problems in medical health is the presence of methicillin resistance (MR) in a broad range of bacteria particularly those contained within, but not limited to, the genus Staphylococcus. MR is conferred by the presence of the gene mecA gene. This gene encodes the penicillin binding protein 2a which prevents the binding of penicillin, methicillin and other penicillin-like antibiotics to enzymes involved in bacterial cell wall biosynthesis. Bacteria containing the mecA gene are thus resistant to a broad spectrum of antibiotics. This resistance to antibiotics is a major factor affecting clinical outcome in hospitals and surgeries. In addition, the mecA gene can be transferred across genera and species of bacteria so that MR is becoming an ever-increasing problem in hospitals, veterinary medicine and agriculture. MR is most commonly determined by biological methods involving the growth of bacteria on antibiotic containing media.

In various embodiments, the invention provides compositions and methods comprising at least one pair of primers and at least one corresponding probe for detecting the presence of nucleic acid sequences associated with antibiotic resistance. In one embodiment, the pair of primers is GATGGTATGTGGAAGTTAGATTG (SEQ ID NO: 44) and GACCGAAACAATGTGGAATTGG (SEQ ID NO: 45). In another embodiment, the pair of primers is GATGGCTATCGTGTCACAATCG (SEQ ID NO: 46) and TTCATATGACGTCTATCCAT (SEQ ID NO: 47). In another embodiment, the pair of primes is GATAAGCATTGGAAATTAGATTG (SEQ ID NO: 80) and AGCTAATTCTATATTGTTTCGGTC (SEQ ID NO: 81). The skilled artisan will understand that these primers are exemplary, and that the length of the primers useful in the invention can be increased or decreased, depending on the reaction conditions and the desired hybridization stringency.

In one embodiment, wherein SEQ ID NO: 44 and SEQ ID NO: 45 is the pair of primers, the corresponding probe is a least one probe selected from the group consisting of CATAGCGTCATTATTCCAG (SEQ ID NO: 48) and CATAGCGTCATTATTCCAGGAATGCAGAA (SEQ ID NO: 49). In another embodiment, wherein SEQ ID NO: 46 and SEQ ID NO: 47 is the pair of primers, the corresponding probe is GATTATGGCTCAGGTACTGCTATC (SEQ ID NO: 50). In another embodiment, wherein SEQ ID NO: 80 and SEQ ID NO: 81 is the pair of primers, the corresponding probe is CCAGACGTAATAGTACCTG (SEQ ID NO: 82). The skilled artisan will understand that these probes are exemplary, and that the length of the probes useful in the invention can be increased or decreased, depending on the reaction conditions and the desired hybridization stringency.

The invention provides methods of determining whether a bacterium present in a biological sample is resistant to an antibiotic, such as methicillin. Generally, the method comprises the steps of obtaining a nucleic acid of a bacterium from a biological sample, generating a nucleic acid amplification product of the nucleic acid and detecting the presence or absence of the amplification product by its ability to hybridize to at least one probe of the invention.

In one embodiment, the invention comprises a diagnostic test to detect a methicillin resistant bacterium. In one embodiment, primers comprising SEQ ID NO: 44 and SEQ ID NO: 45 and probes comprising the sequence selected from the group consisting of SEQ ID NO: 48 and SEQ ID NO: 49 are used to detect methicillin resistant bacteria. In one embodiment, real time PCR is used to amplify nucleotide sequences isolated from bacteria. In this embodiment, observing the detection of amplified products using the probe selected from the group consisting of SEQ ID NO: 48 and SEQ ID NO: 49 identifies the bacteria as being methicillin resistant. In another embodiment, the lack of detection of amplified products using the probe selected from the group consisting of SEQ ID NO: 48 and SEQ ID NO: 49, identifies the bacteria as not being methicillin resistant.

In another embodiment, primers comprising SEQ ID NO: 46 and SEQ ID NO: 47 and probe comprising SEQ ID NO: 50 are used to detect methicillin resistant bacteria. In one embodiment, real time PCR is used to amplify nucleotide sequences isolated from bacteria. In this embodiment, observing the detection of amplified products using the probe, SEQ ID NO: 50, identifies the bacteria as being methicillin resistant. In another embodiment, the lack of detection of amplified products using the probe, SEQ ID NO: 50, identifies the bacteria as not being methicillin resistant.

In another embodiment, primers comprising SEQ ID NO: 80 and SEQ ID NO: 81 and probe comprising SEQ ID NO: 82 are used to detect methicillin resistant bacteria. In one embodiment, real time PCR is used to amplify nucleotide sequences isolated from bacteria. In this embodiment, observing the detection of amplified products using the probe, SEQ ID NO: 82, identifies the bacteria as being methicillin resistant. In another embodiment, the lack of detection of amplified products using the probe, SEQ ID NO: 82, identifies the bacteria as not being methicillin resistant.

### Species Identification

Rapid determination of the species of bacteria present in a biological sample is important for a variety of reasons, including determining the optimal treatment regimen to apply to an infected subject. Thus, the invention provides compositions and methods comprising at least one pair of primers and at least one corresponding probe for determining the presence of nucleic acid sequences associated with a particular bacteria species. In various embodiments, the pair of primers useful in the methods of the invention is at least one pair of primers selected from: AGAGTTTGATCHTGGCTCAG (SEQ ID NO: 83) and CCYACTGCTGCCTCCCGTA (SEQ ID NO: 84); GGGAHGAMGWCAARTCATCAT (SEQ ID NO: 85) and CGATTACTAGCGATTCCRRCTTC (SEQ ID NO: 86); and GAAGYYGGAATCGCTAGTAATCG (SEQ ID NO: 87) and TACRGHTACCTTGTTACGACT (SEQ ID NO: 88). The skilled artisan will understand that these primers are exemplary, and that the length of the primers useful in the invention can be increased or decreased, depending on the reaction conditions and the desired hybridization stringency.

In one embodiment, the corresponding probe is GAAAACAATGGCGCA (SEQ ID NO: 89) for the identification of Enterobacter cloacae. In another embodiment, the corresponding probe is ACGGTTACCACGGAG (SEQ ID NO: 90) for the identification of Pseudomonas aeruginosa. In another embodiment, the corresponding probe is CTAGCCTAACTGCAAAGA (SEQ ID NO: 91) for the identification of Acinetobacter baumannii. In another embodiment, the corresponding probe is GATGACCGCCACACT (SEQ ID NO: 92) for the identification of Citrobacter koseri. In another embodiment, the corresponding probe is GCCTCTTGCCATCA (SEQ ID NO: 93) for the identification of Citrobacter koseri. In another embodiment, the corresponding probe is GCCTCATGCCATCA (SEQ ID NO: 94) for the identification of Klebsiella pneumonia. In another embodiment, the corresponding probe is GGCAGATACAAAGAG (SEQ ID NO: 95) for the identification of Proteus mirabilis. In another embodiment, the corresponding probe is GGCGTATACAAAGGG (SEQ ID NO: 96) for the identification of Morganella moranii. In another embodiment, the corresponding probe is GATCTGCCTGATGGC (SEQ ID NO: 97) for the identification of Morganella moranii. In another embodiment, the corresponding probe is AACGAGTCGCTAGAC (SEQ ID NO: 98) for the identification of Enterococcus faecalis. In another embodiment, the corresponding probe is AACGAGTTGCGAAGT (SEQ ID NO: 99) for the identification of Enterococcus faecium. In another embodiment, the corresponding probe is ACCGTAAGGAGCCAG (SEQ ID NO: 100) for the identification of Streptococcus pneumonia. In another embodiment, the corresponding probe is CCTATTAGGAGCCAG (SEQ ID NO: 101) for the identification of Streptococcus pyogenes. In another embodiment, the corresponding probe is TTCTCTGATGTTAGC (SEQ ID NO: 102) for the identification of Staphylococcus aureus. In another embodiment, the corresponding probe is TCCTCTGACGTTAGC (SEQ ID NO: 103) for the identification of Staphylococcus epidermidis. In another embodiment, the corresponding probe is CAACGTTTCCAAAGGA (SEQ ID NO: 104) for the identification of Pseudomonas putida. In another embodiment, the corresponding probe is TAGCACAGAGGAGCTT (SEQ ID NO: 105) for the identification of Citrobacter freundii. In another embodiment, the corresponding probe is TAGCACAGAGGAGGCTT (SEQ ID NO: 106) for the identification of Enterobacter sakazakii. In another embodiment, the corresponding probe is CAGCTTGCTGCTTCGCT (SEQ ID NO: 107) for the identification of Klebsiella terrigena. In another embodiment, the corresponding probe is TTGTAGGTGAGGTAAC (SEQ ID NO: 108) for the identification of Klebsiella oxytoca. In another embodiment, the corresponding probe is TAGCACAAGGGAGCTTG (SEQ ID NO: 109) for the identification of Serratia marcescens. In another embodiment, the corresponding probe is GGACCCGCGCCGTATT (SEQ ID NO: 110) for the identification of Staphylococcus lugdunensis. In another embodiment, the corresponding probe is ATCTCTTAGGAGCAAAGC (SEQ ID NO: 111) for the identification of Providecia stuartii. In another embodiment, the corresponding probe is CTGAGGTTTGGTGTTTA (SEQ ID NO: 112) for the identification of Streptococcus agalactiae.

The skilled artisan will understand that any combination of primer pairs, and any combination of probes, can be used to analyze a sample to identify the one or more species present in a biological sample. The skilled artisan will understand that the primers and probes described herein are exemplary, and that the length of the primers and probes useful in the invention can be increased or decreased, depending on the reaction conditions and the desired hybridization stringency.

Staphylococcus aureus

Staphylococcus aureus is the most common species of staphylococci to cause staph infections. Staphylococcus aureus can cause a range of illnesses from minor skin infections to life threatening diseases such as pneumonia, meningitis, osteomyelitis, endocarditis, toxic shock syndrome, bacteremia, and sepsis. Detecting the presence of Staphylococcus aureus is important for determining the optimal treatment regimen. In addition, antibiotic resistance in some strains of Staphylococcus aureus has made treatment with standard antibiotics ineffective in these infections. For example, methicillin-resistant Staphylococcus aureus (MRSA) is resistant to many antibiotics.

The invention provides compositions and methods comprising at least one pair of primers and at least one corresponding probe for detecting the presence of nucleic acid sequences associated with Staphylococcus aureus. In various embodiments, the pair of primers is AGAGTTTGATCHTGGCTCAG (SEQ ID NO: 51) and CCTACTGCTGCCTCCCTGTA (SEQ ID NO: 52). The skilled artisan will understand that these primers are exemplary, and that the length of the primers useful in the invention can be increased or decreased, depending on the reaction conditions and the desired hybridization stringency.

In one embodiment, the corresponding probe is TTCTCTGATGTTAGC (SEQ ID NO: 53). The skilled artisan will understand that these probes are exemplary, and that the length of the probes useful in the invention can be increased or decreased, depending on the reaction conditions and the desired hybridization stringency.

The invention provides methods of determining if a bacterium present in a biological sample is Staphylococcus aureus. Generally, the method comprises the steps of obtaining a nucleic acid of a bacterium from a biological sample, generating a nucleic acid amplification product of the nucleic acid and detecting the presence or absence of the amplification product by its ability to hybridize to a probe of the invention.

In one embodiment, the invention comprises a diagnostic test to detect Staphylococcus aureus. In this embodiment, primers comprising SEQ ID NO: 51 and SEQ ID NO: 52 and probes comprising SEQ ID NO: 53 are used to detect Staphylococcus aureus. In one embodiment, real time PCR is used to amplify nucleotide sequences isolated from bacteria. In this embodiment, observing the detection of amplified products using the probe, SEQ ID NO: 53, identifies the bacteria as Staphylococcus aureus. In another embodiment, the lack of detection of amplified products using the probe, SEQ ID NO: 53, identifies the bacteria as not being Staphylococcus aureus.

Staphylococcus epidermidis

Staphylococcus epidermidis can cause infection in patients, especially those with compromised immune systems. Staphylococcus epidermidis produces a biofilm and is one of the most prevalent bacteria found on catheters and other implanted medical devices. As with other species, there are strains of Staphylococcus epidermidis that have developed antibiotic resistance, including methicillin resistance.

The invention provides compositions and methods comprising at least one pair of primers and at least one corresponding probe for detecting the presence of nucleic acid sequences associated with Staphylococcus epidermidis. In various embodiments, the pair of primers is AGAGTTTGATCHTGGCTCAG (SEQ ID NO: 54) and CCTACTGCTGCCTCCCTGTA (SEQ ID NO: 55). The skilled artisan will understand that these probes are exemplary, and that the length of the probes useful in the invention can be increased or decreased, depending on the reaction conditions and the desired hybridization stringency.

In various embodiments, the corresponding probe is TCCTCTGACGTTAGC (SEQ ID NO: 56). The skilled artisan will understand that this probe is exemplary, and that the length of the probe useful in the invention can be increased or decreased, depending on the reaction conditions and the desired hybridization stringency.

The invention provides methods of determining if a bacterium present in a biological sample is Staphylococcus epidermidis. Generally, the method comprises the steps of obtaining a nucleic acid of a bacterium from a biological sample, generating a nucleic acid amplification product of the nucleic acid and detecting the presence or absence of the amplification product by its ability to hybridize to a probe of the invention.

In one embodiment, the invention comprises a diagnostic test to detect Staphylococcus epidermidis. In this embodiment, primers comprising SEQ ID NO: 54 and SEQ ID NO: 55 and probes comprising SEQ ID NO: 56 are used to detect Staphylococcus epidermidis. In one embodiment, real time PCR is used to amplify nucleotide sequences isolated from bacteria. In this embodiment, observing the detection of amplified products using the probe, SEQ ID NO: 56, identifies the bacteria as Staphylococcus epidermidis. In another embodiment, the lack of detection of amplified products using the probe, SEQ ID NO: 56, identifies the bacteria as not being Staphylococcus epidermidis.

### Combination Assays

It should be understood that any combination of any two or more assays, involving any one or more primer pairs and any one or more probes, in whole or in part, described elsewhere herein, can be used concurrently or sequentially to determine the identity, as well as other multiple characteristics of a particular bacterium present in a sample. By way of non-limiting examples, the combination assays of the invention can be used to determine the gram status of a bacterium in a sample, the methicillin resistance status of a bacterium in a sample, the species of a bacterium in a sample, and any combination thereof.

In one embodiment, the invention comprises compositions and methods of detecting at least one characteristic of a bacterium present in a biological sample. Generally, the method comprises the steps of obtaining a nucleic acid of a bacterium from a biological sample, dividing the isolated nucleic acid material into multiple test samples, generating nucleic acid amplification products of the nucleic acid in each of the test samples and detecting the presence or absence of at least one amplification product by its ability to hybridize to at least one probe of the invention. In some embodiments, the invention comprises compositions and methods of detecting at least two characteristics of a bacterium present in a biological sample. Generally, the method comprises the steps of obtaining a nucleic acid of a bacterium from a biological sample, dividing the isolated nucleic acid material into multiple test samples, generating nucleic acid amplification products of the nucleic acid in each of the test samples and detecting the presence or absence of at least two amplification products by its ability to hybridize to at least two probes of the invention.

In one embodiment, test samples of isolated nucleic acid are probed to determine both the gram status of a bacterium and the methicillin-resistance status of the bacteria. In one embodiment, a portion of a sample of isolated nucleic acid is amplified using SEQ ID NO: 40 and SEQ ID NO: 41 as a primer set, and the amplified products are probed using at least one of SEQ ID NO: 42 and SEQ ID NO: 43 to determine gram status, and another portion of the sample of the isolated nucleic acid is amplified using SEQ ID NO: 44 and SEQ ID NO: 45 as a primer set, and the amplified products are probed using at least one of SEQ ID NO: 48 and SEQ ID NO: 49 to determine methicillin resistance. In another embodiment, a portion of a sample of isolated nucleic acid is amplified using SEQ ID NO: 40 and SEQ ID NO: 41 as a primer set, and the amplified products are probed using at least one of SEQ ID NO: 42 and SEQ ID NO: 43 to determine gram status, and another portion of the sample of the isolated nucleic acid is amplified using SEQ ID NO: 46 and SEQ ID NO: 47 as a primer set, and the amplified products are probed using SEQ ID NO: 50 to determine methicillin resistance.

In another embodiment, test samples of isolated nucleic acid are probed to determine both the methicillin-resistance status of the bacterium and whether the bacterium is Staphylococcus aureus. In one embodiment, a portion of a sample of the isolated nucleic acid is amplified using SEQ ID NO: 44 and SEQ ID NO: 45 as a primer set, and the amplified products are probed using at least one of SEQ ID NO: 48 and SEQ ID NO: 49 to determine methicillin resistance status, and another portion of the sample of the isolated nucleic acid is amplified using SEQ ID NO: 51 and SEQ ID NO: 52 as a primer set, and the amplified products are probed using SEQ ID NO: 53 to determine whether the bacterium is Staphylococcus aureus. In this embodiment, the invention provides compositions and methods of determining whether a bacterium is Methicillin-resistant Staphylococcus aureus (MRSA). In another embodiment, a portion of a sample of the isolated nucleic acid is amplified using SEQ ID NO: 46 and SEQ ID NO: 47 as a primer set, and the amplified products are probed using SEQ ID NO: 50 to determine methicillin resistance status, and another portion of the sample of the isolated nucleic acid is amplified using SEQ ID NO: 51 and SEQ ID NO: 52 as a primer set, and the amplified products are probed using SEQ ID NO: 53 to determine whether the bacterium is Staphylococcus aureus. In this embodiment, the invention provides compositions and methods of determining whether a bacterium is Methicillin-resistant Staphylococcus aureus (MRSA).

### Kits

A kit is envisaged comprising the compositions used in every method disclosed herein. The following description of kits useful for the amplification and identification of nucleic acid sequences in the genome of a bacterium isolated from a sample is not intended to be limiting and should not be construed that way.

The kits of the invention comprise a negative control and a positive control. The kits also comprise at least one primer pair and at least one corresponding probe. The kits optionally can include at least one sample container for containing a sample having a nucleic acid of an unknown bacterium. Furthermore, the kits include an instructional material for use in the amplification and identification of nucleic acid sequences in the genome of a bacterium isolated from a sample. The instructional material can be a publication, a recording, a diagram, or any other medium of expression which can be used to communicate the usefulness of the method of the invention. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Using Real-Time PCR to Determine the Gram Status of a Bacterium

The materials and methods used in this example are now described.

The reaction mixture for real time PCR included 5 μl of H₂O, the appropriate primers and probe, and 5 μl Taqman 2× Universal Master Mix (ABI). In the reaction mixture, the final concentration of each primer was 800 nM and the final concentration of the probe was 250 nM. The primer set was SEQ ID NO: 40 and SEQ ID NO: 41. The probe in the reaction mixture was either SEQ ID NO: 42 or SEQ ID NO: 43.

To make the reaction mixture for 10 reactions (100 μl final volume of reaction mixture), 0.8 μl of 100 μM stock solutions of each primer were added with 0.25 μl of 100 μM stock solution of minor groove binder probe and H₂O to give a final volume of 50 μl. To this solution, 50 μl of Taqman 2× Universal Master Mix was added. The final solution was vortexed, and 10 μl of the mixture was placed into one well of a 96 well plate. Before running the reaction, 1 μl of bacterial DNA (0.5 ng/μl) was added. DNA was extracted from bacteria by mixing 250 μl of bacterial culture with 250 μl of Bacterial Lysis Buffer (Roche: MagNA Pure Bacterial Lysis Buffer) and placing mixture into MagNA Lyser Green Beads tubes (Roche). Cells were disrupted using a MagNA Lyser Instrument (Roche) set at 6000 rpm for 30 seconds. After recovery, the bacterial lysate was processed using a MagNA Pure Compact instrument (Roche) and reagents supplied in the MagNA Pure Compact Nucleic Acid Isolation Kit I (Roche).

The 96 well plate containing the reaction mixtures was placed into an ABI 790 HT real time PCR machine. A standard real time PCR program was used, subjecting the samples to the following thermal cycle steps:

1. 50° C. for 2 minutes

2. 95° C. for 10 minutes

3. 95° C. for 15 seconds

4. 60° C. for 1 minute

Steps 3 and 4 were repeated for 35 cycles. Collected data was analyzed using software supplied with the machine.

The probe was labeled with a 5′ fluorophor (6-FAM) having a 3′ quencher (black hole) built into the end of the probe.

The results of this example are now described.

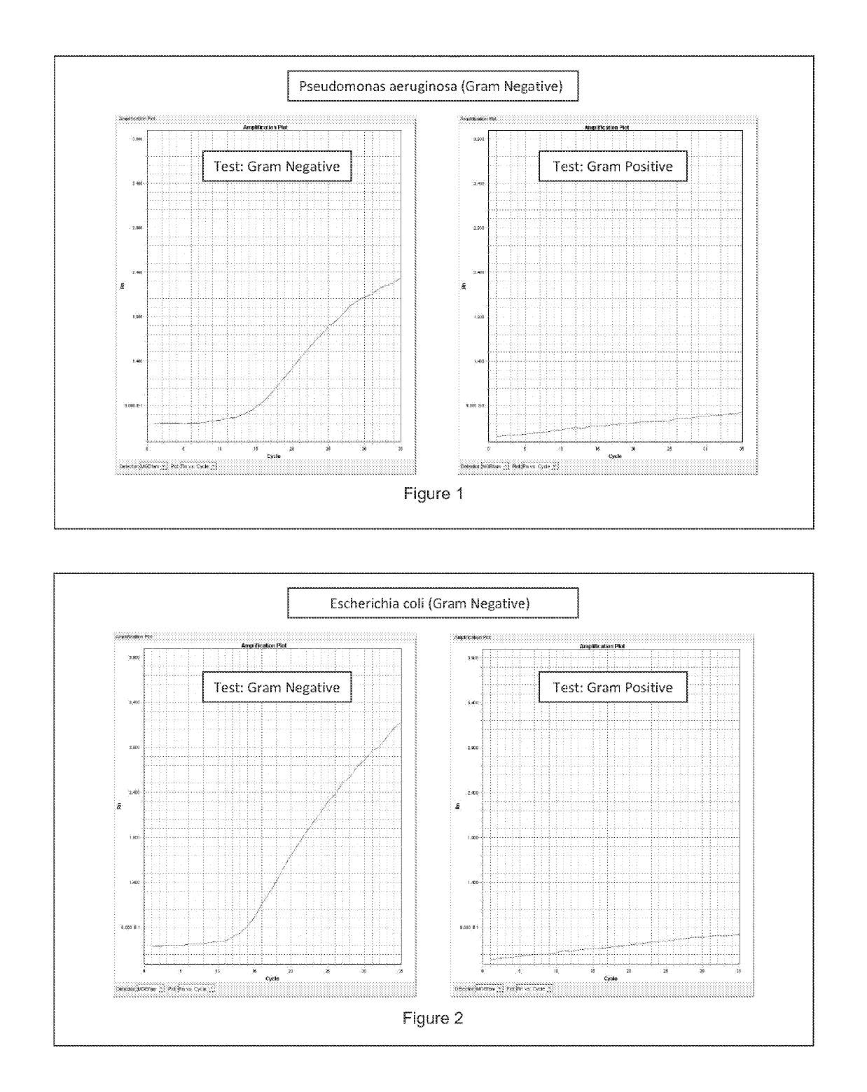

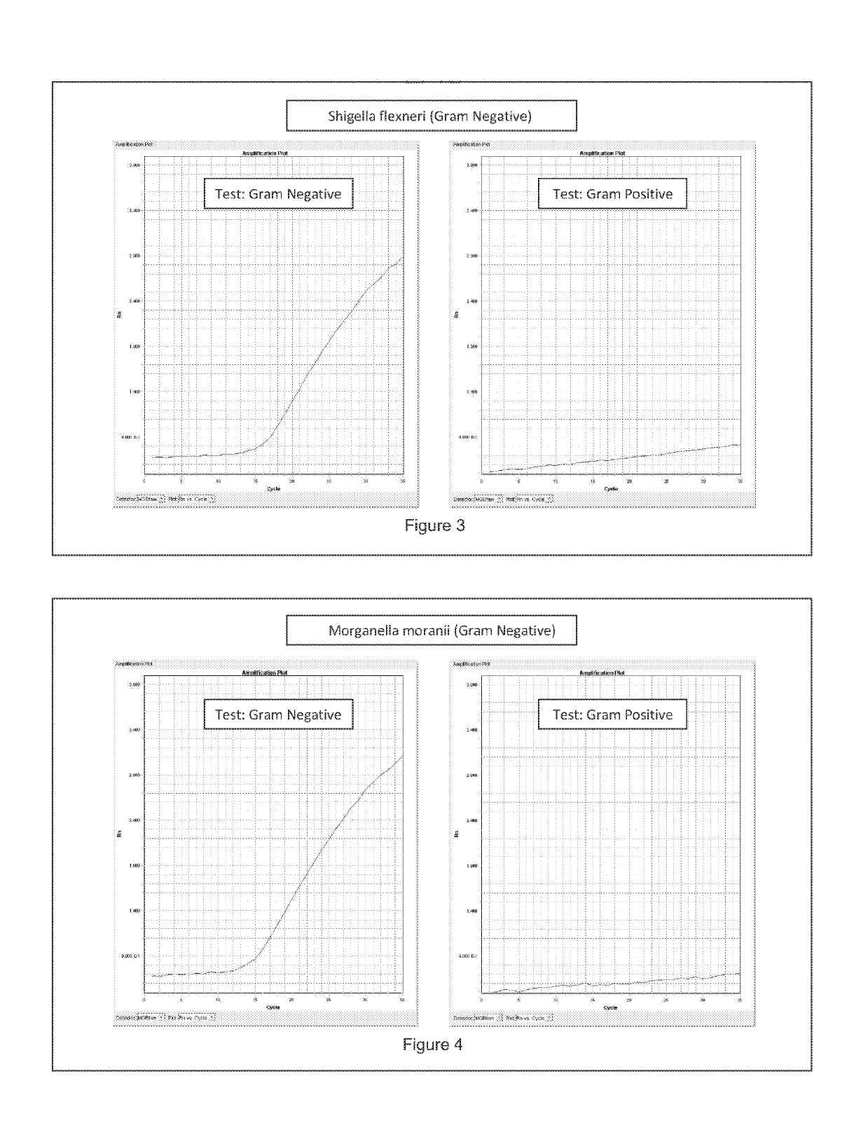

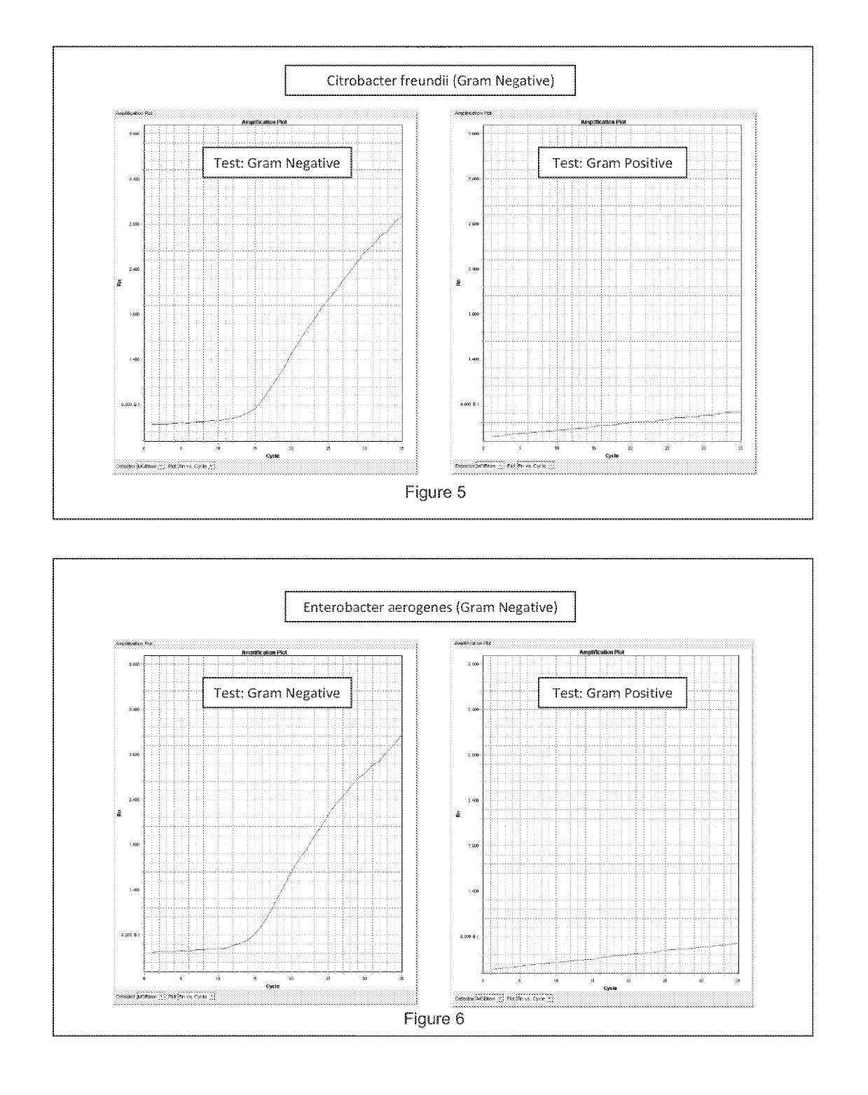

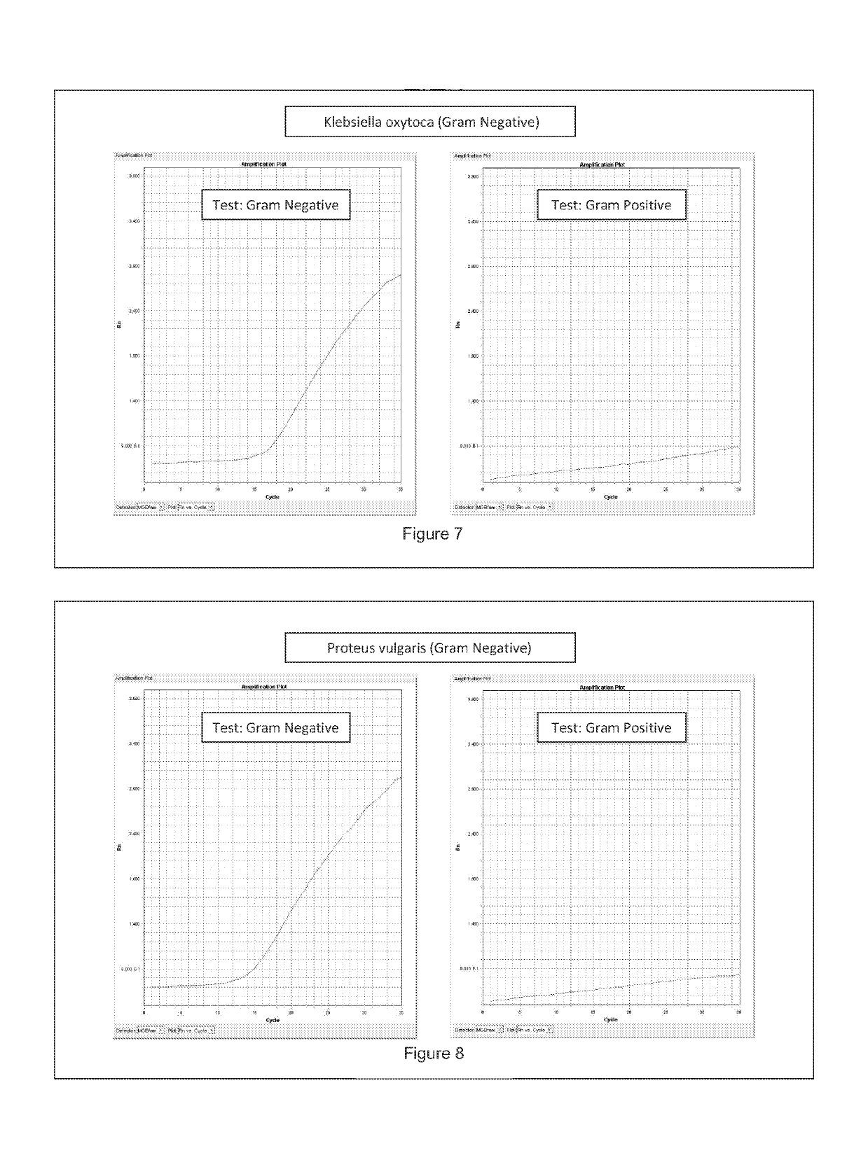

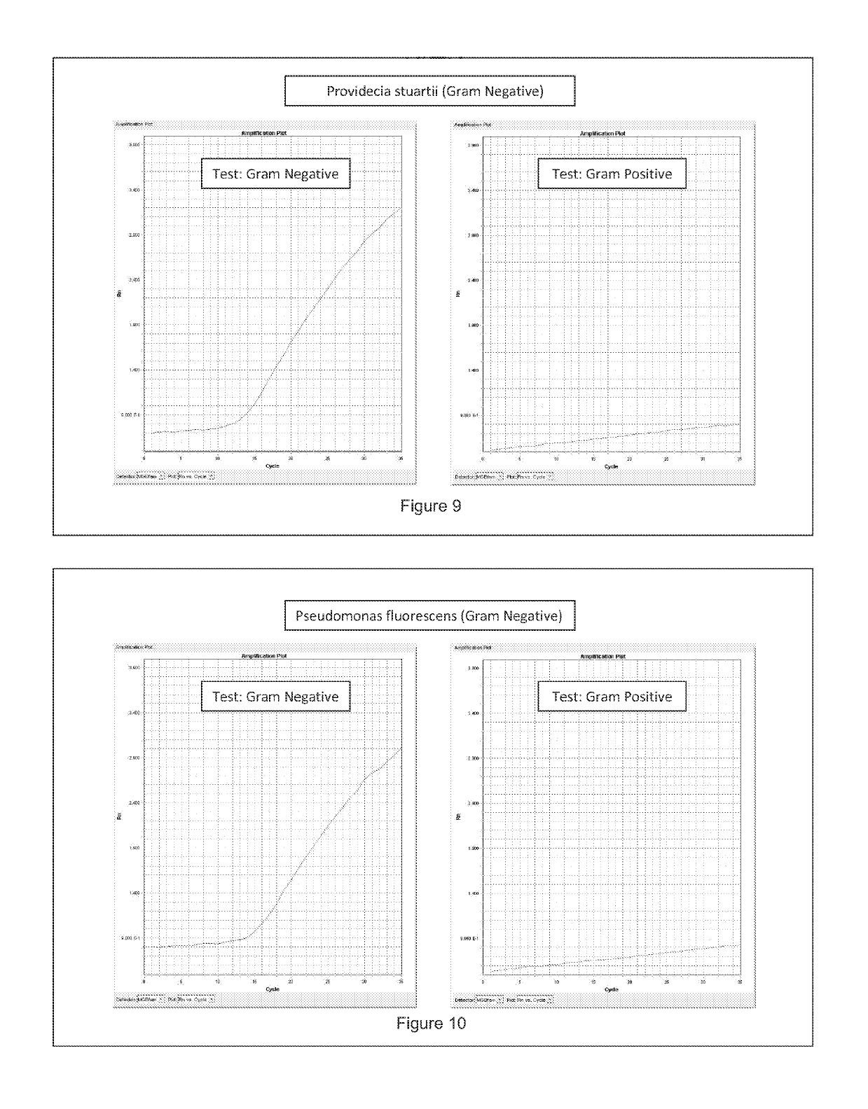

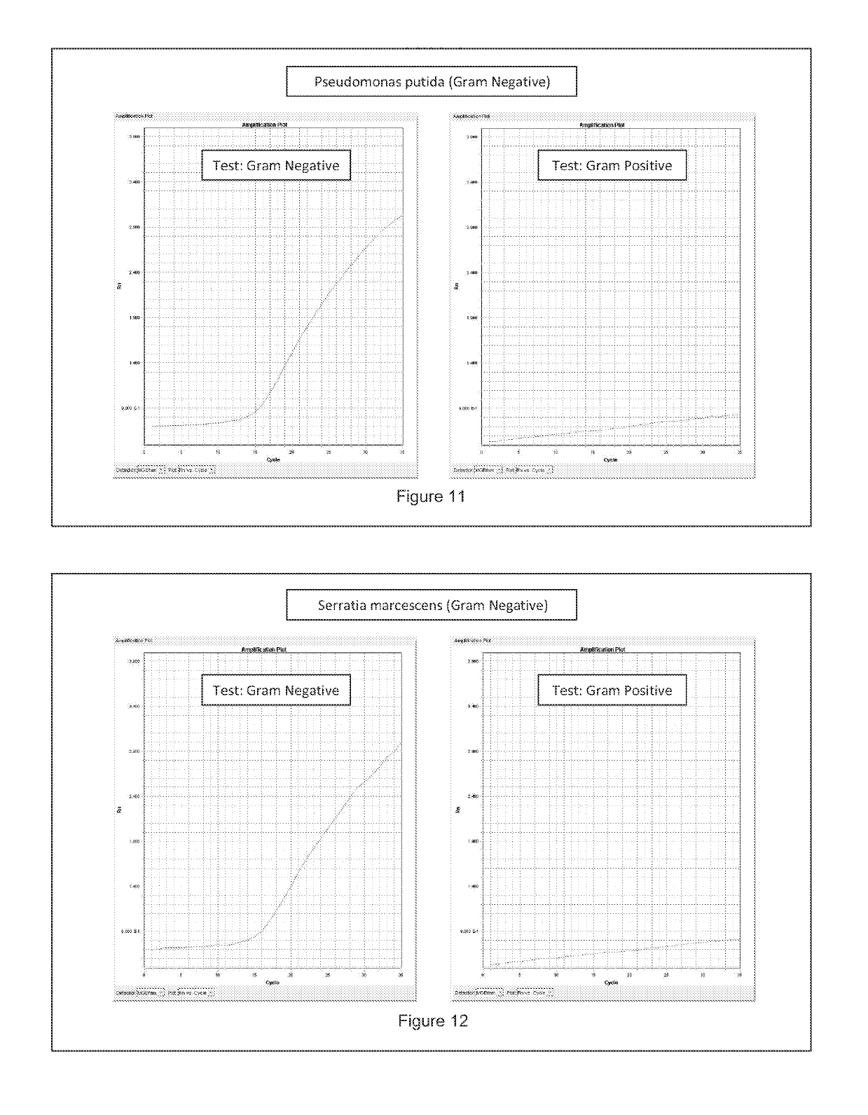

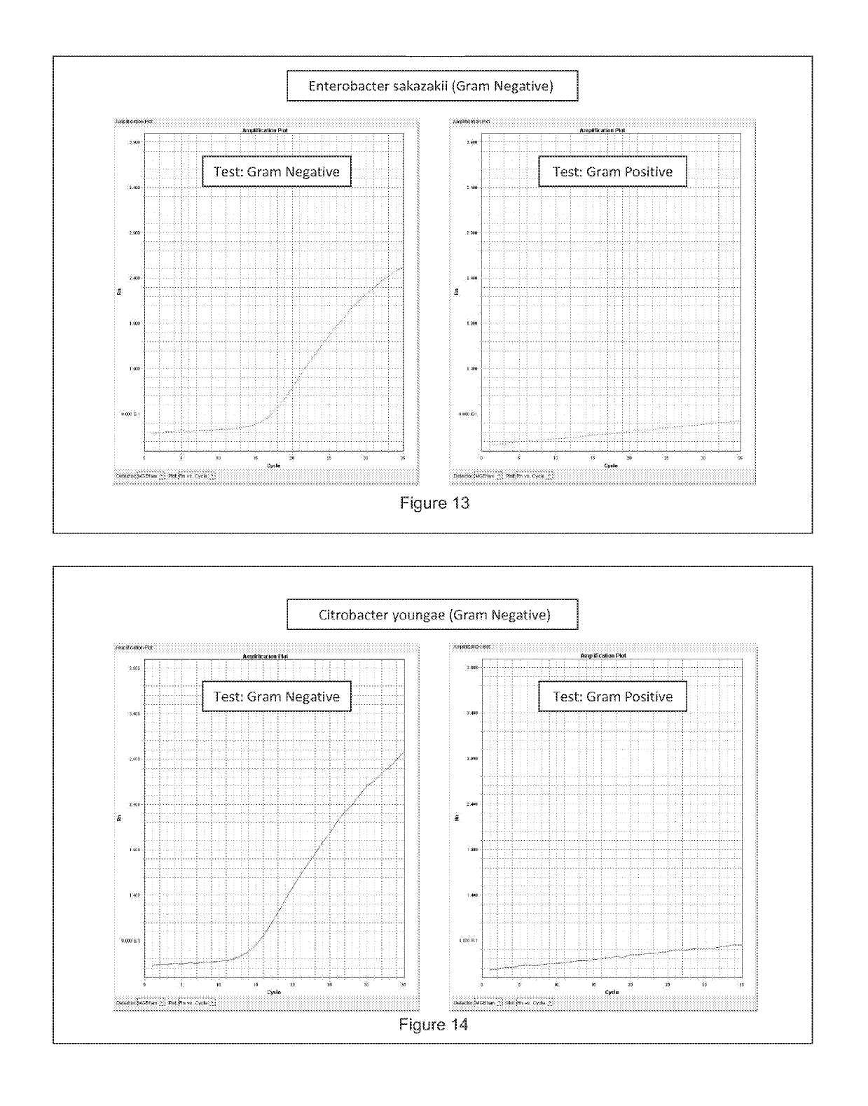

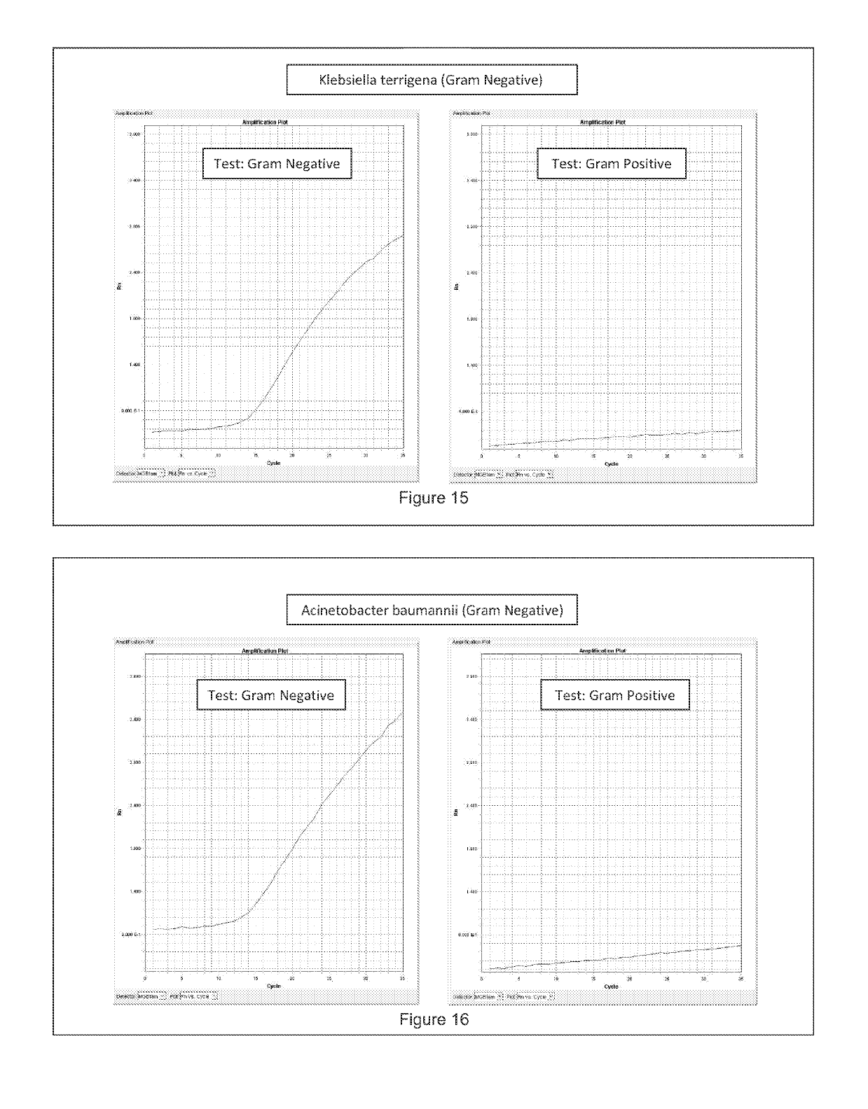

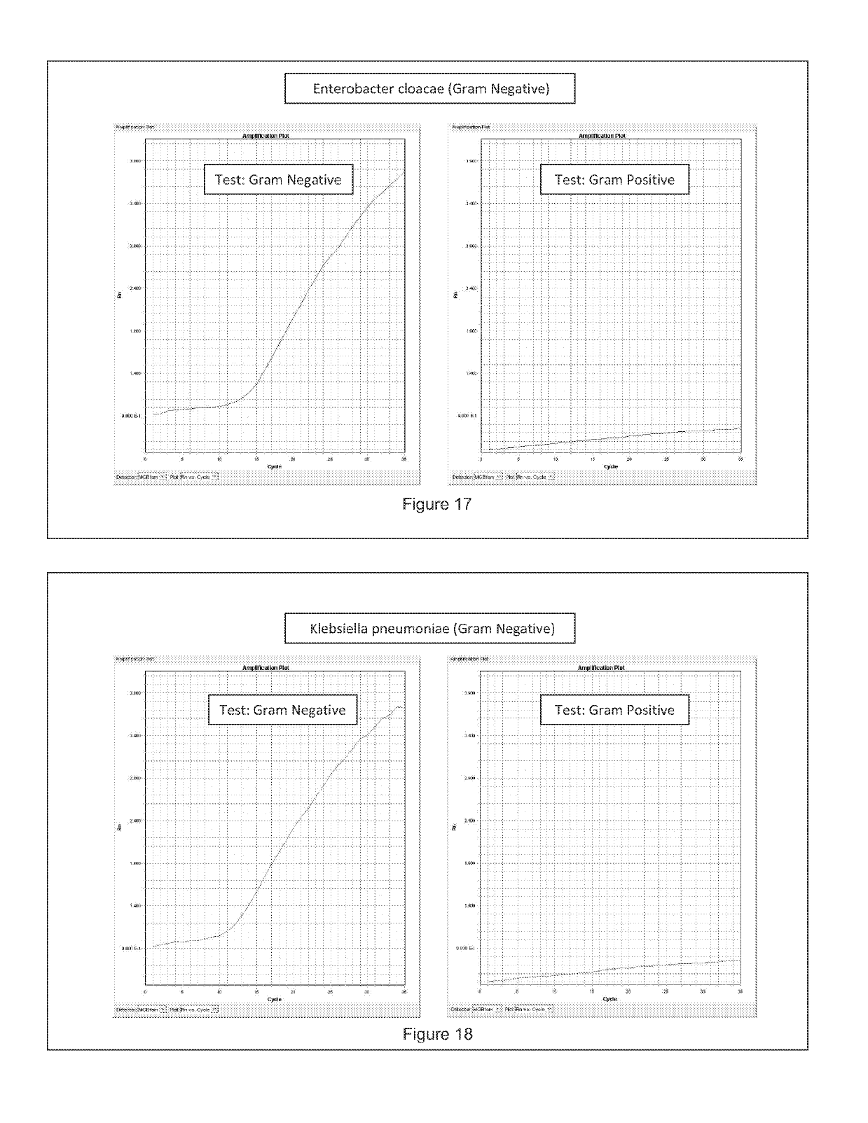

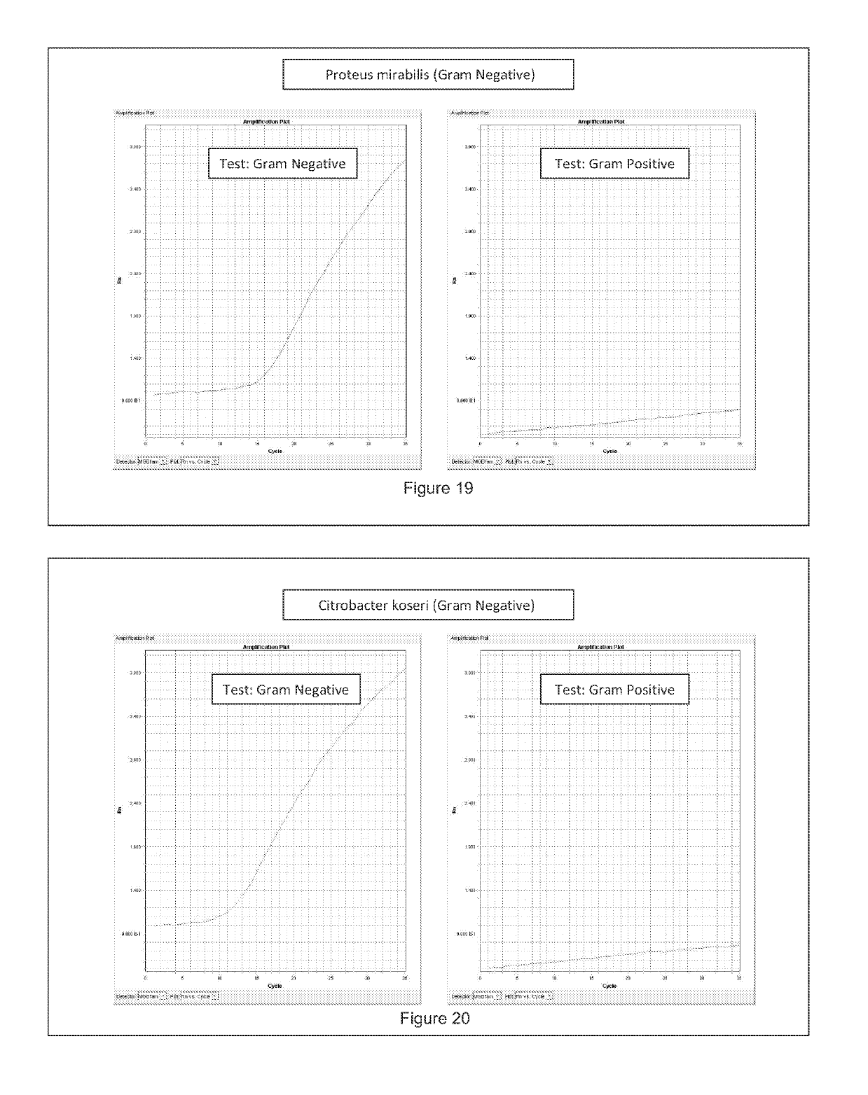

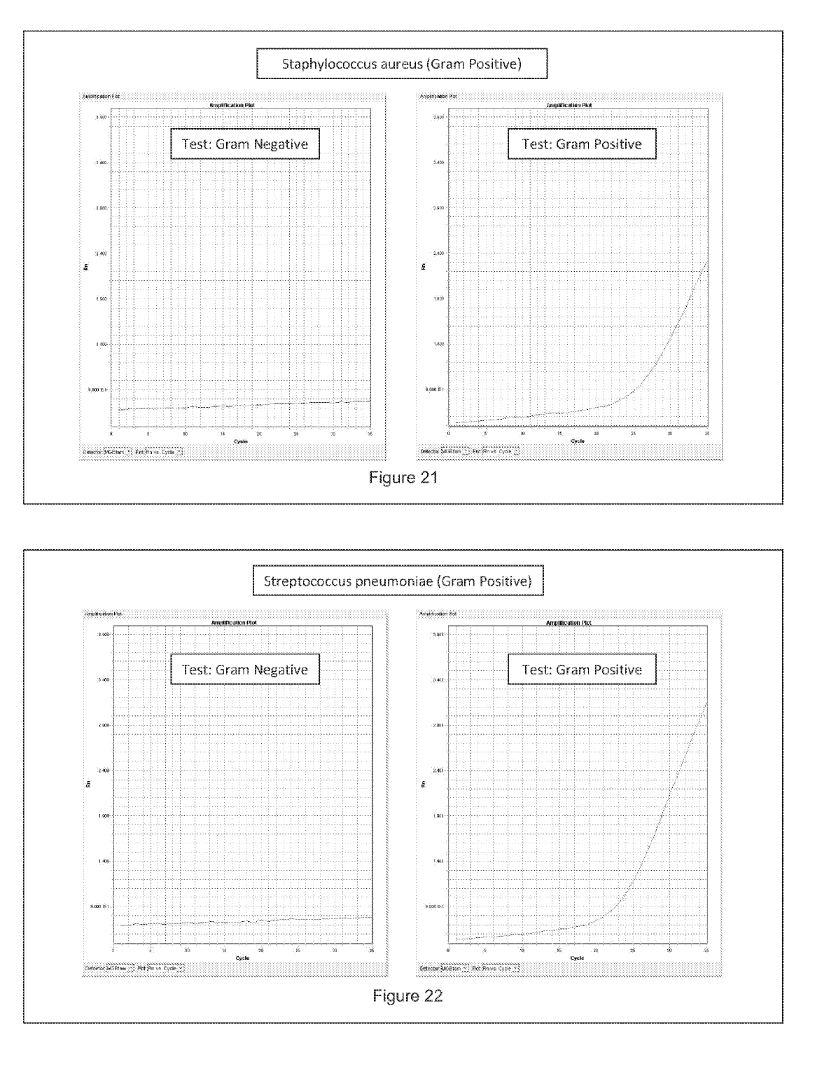

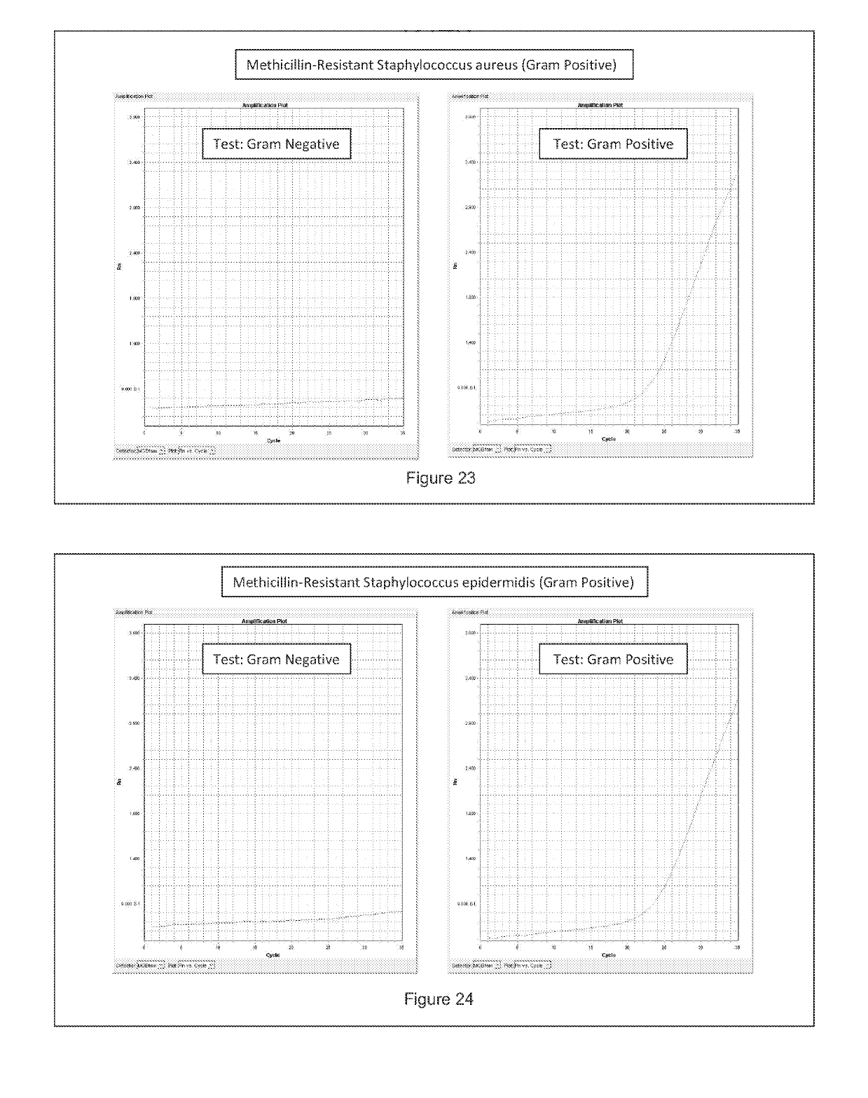

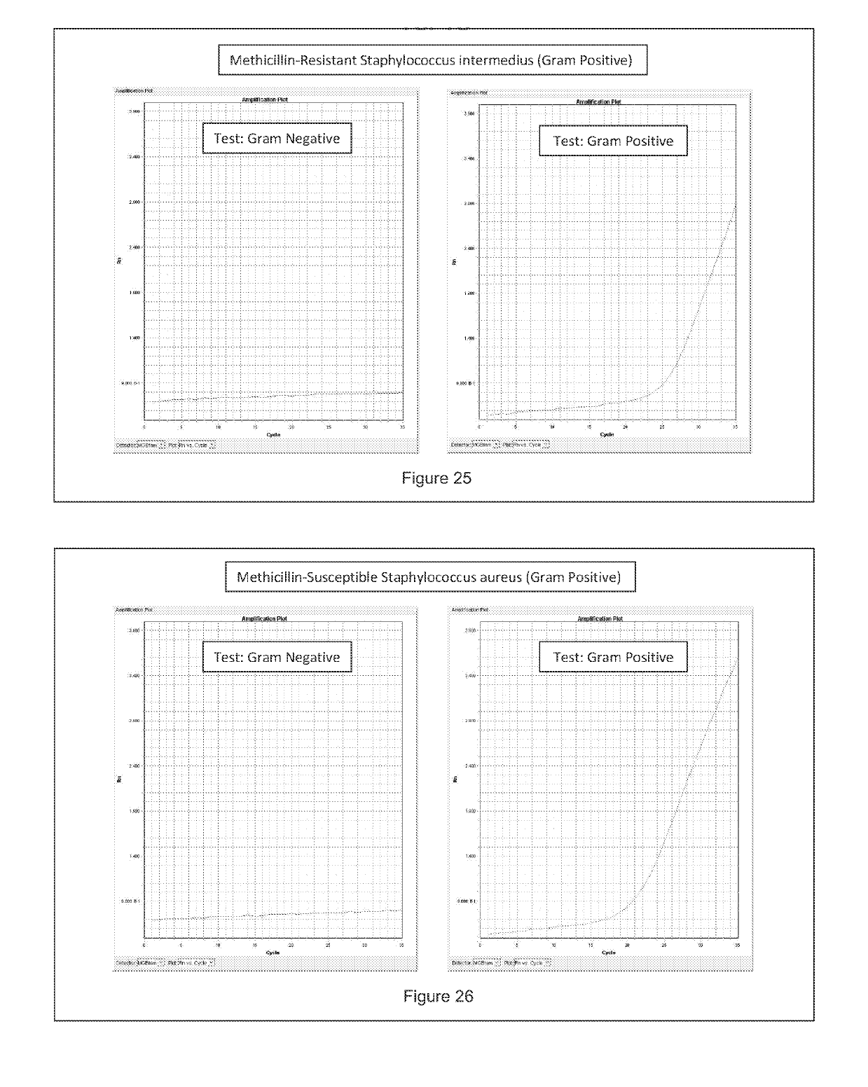

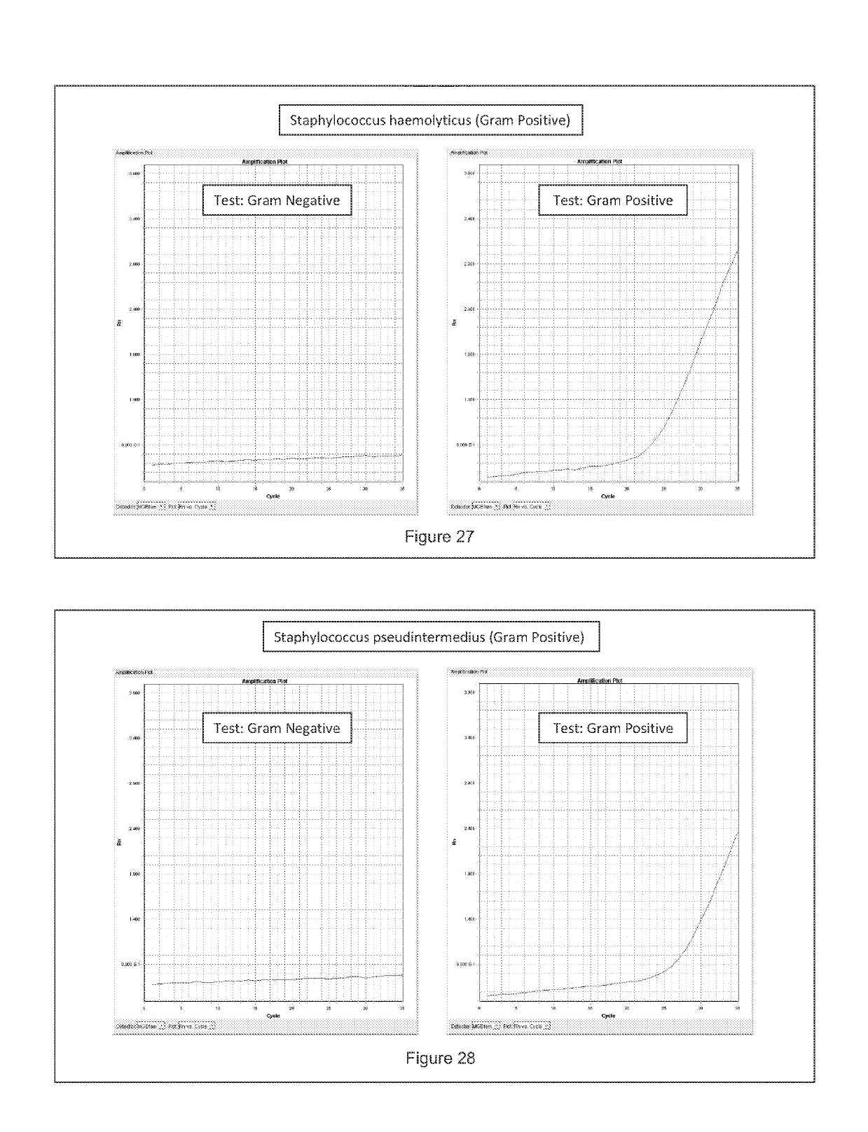

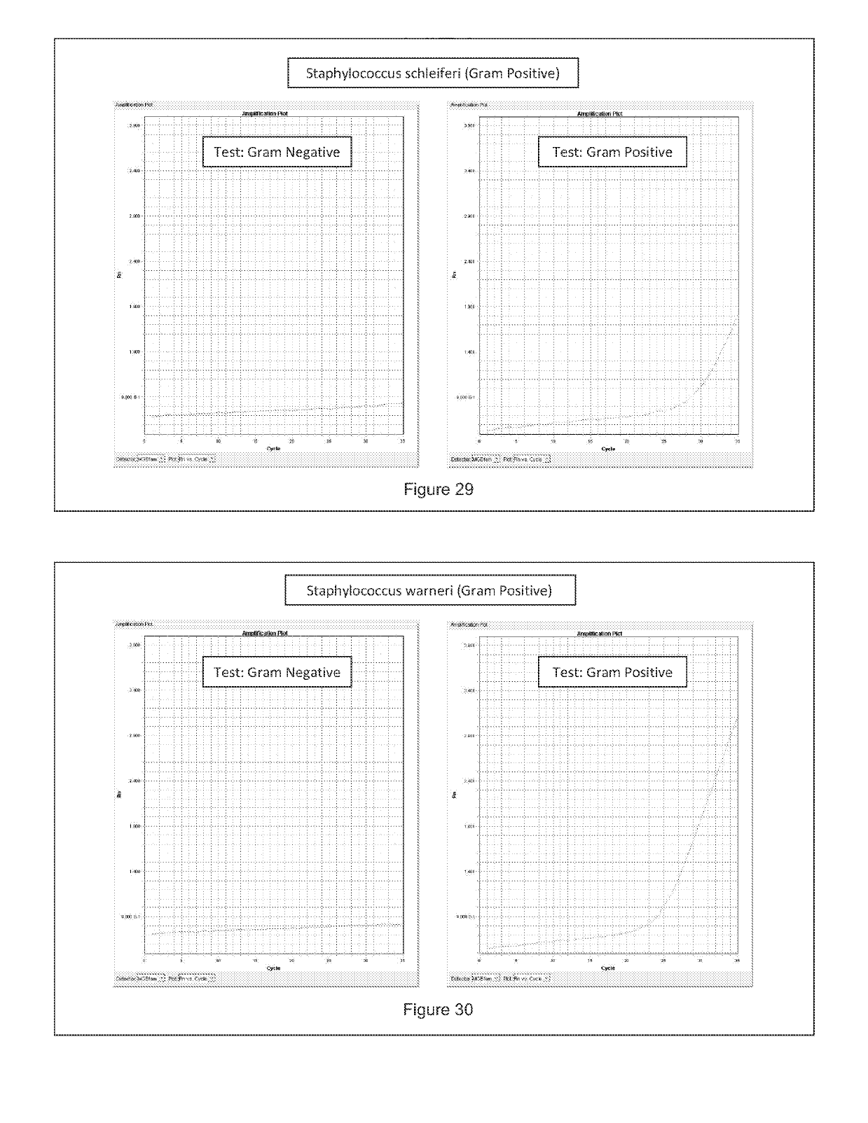

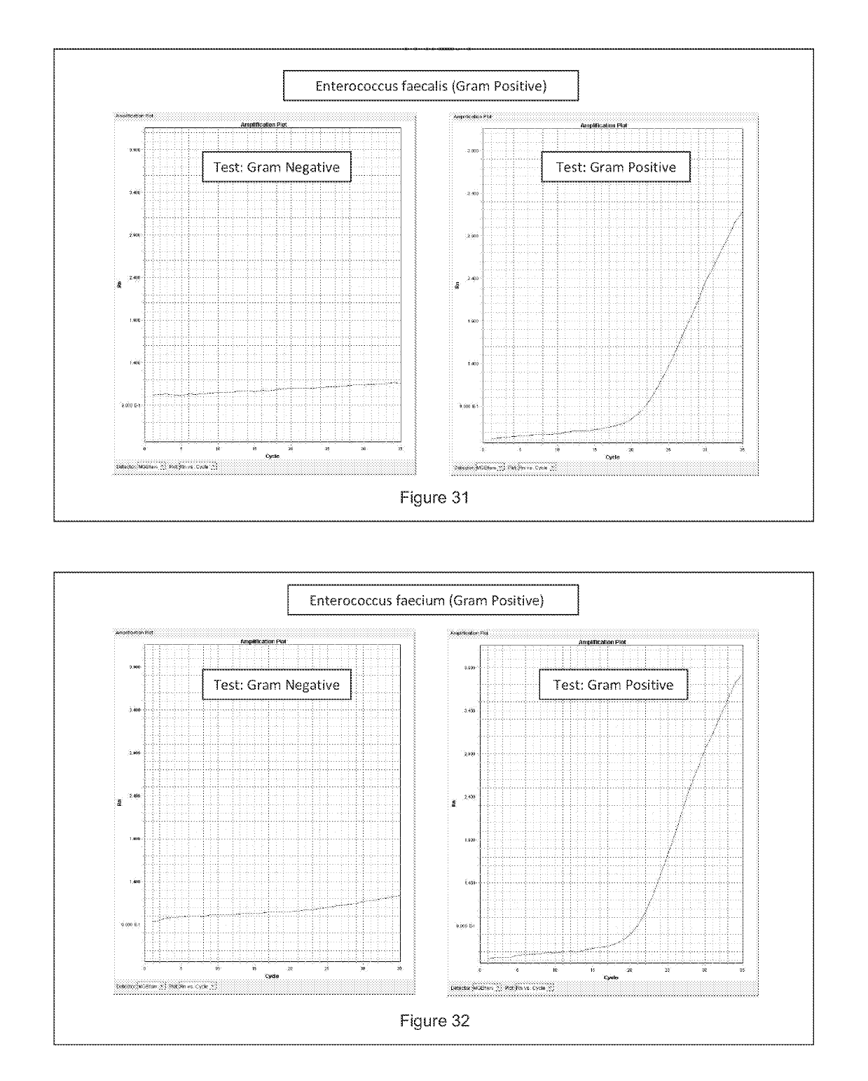

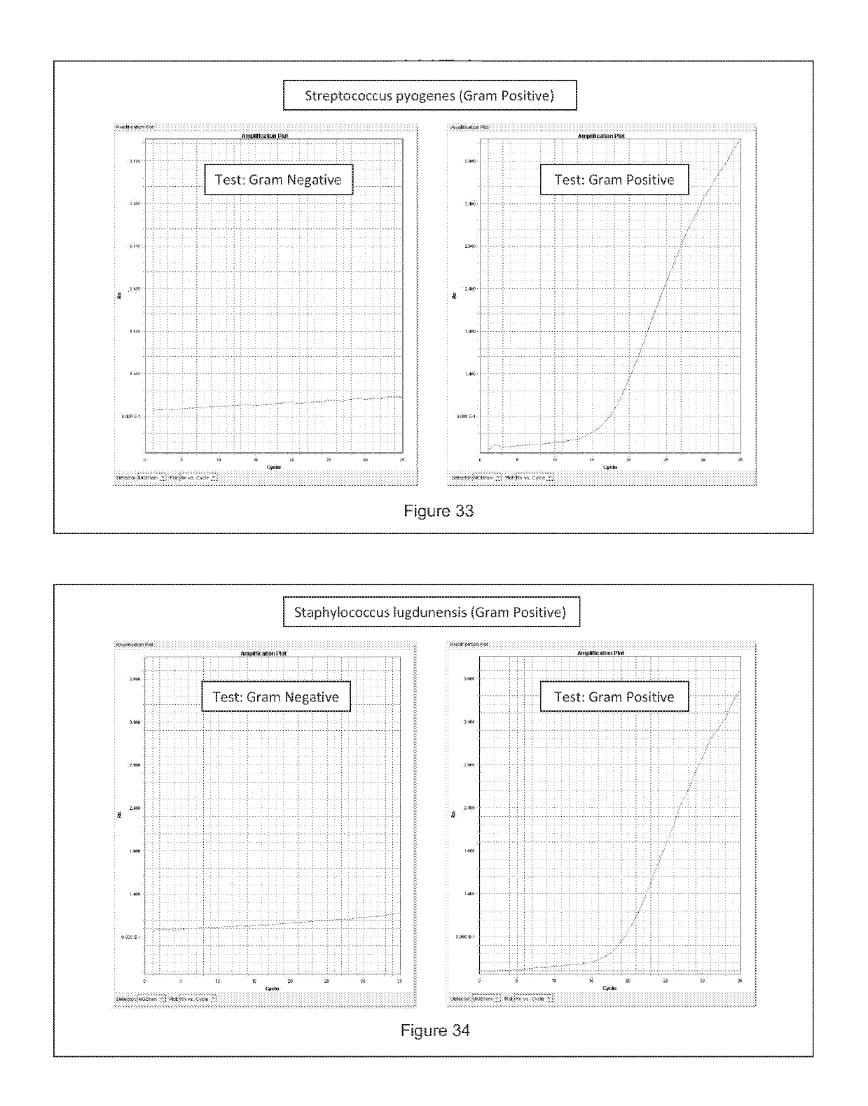

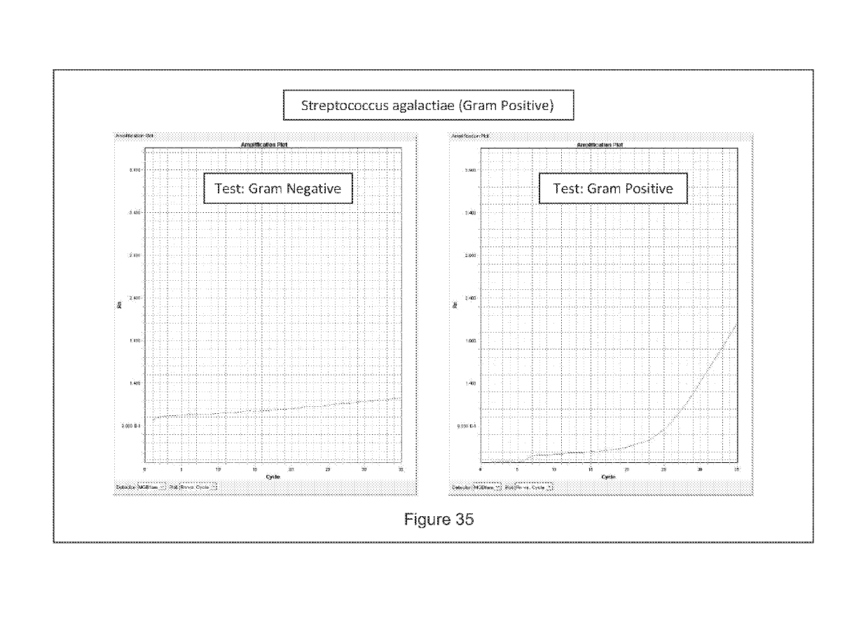

Bacterial DNA from various different species was subjected to real time PCR, using SEQ ID NO: 40 and SEQ ID NO: 41 as the primer pair and either SEQ ID NO: 42 or SEQ ID NO: 43 as the probe. In all cases of known gram negative bacteria, detection of amplified products was observed when the probe in the reaction mixture was SEQ ID NO: 43, but was not observed when the probe in the reaction mixture was SEQ ID NO: 42 (FIGS. 1-20). Conversely, in all cases of known gram positive bacteria, detection of amplified products was observed when the probe in the reaction mixture was SEQ ID NO: 42, but was not observed when the probe in the reaction mixture was SEQ ID NO: 43 (FIGS. 21-35).

This data shows SEQ ID NO: 40 and SEQ ID NO: 41 can be used as primers to produce amplification products from nucleic acid isolated from a large variety of bacteria, all of which can be probed with SEQ ID NO: 42 to determine whether an unknown bacteria is gram positive or gram negative, with the detection of amplified products with SEQ ID NO: 42 indicating that the unknown bacteria is gram positive and the lack of detection of amplified products with SEQ ID NO: 42 indicating that the unknown bacteria is gram negative.

Additionally, this data shows that SEQ ID NO: 40 and SEQ ID NO: 41 can be used as primers to produce amplification products from nucleic acid isolated from a large variety of bacteria, all of which can be probed with SEQ ID NO: 43 to determine whether an unknown bacteria is gram positive or gram negative, with the detection of amplified products with SEQ ID NO: 43 indicating that the unknown bacteria is gram negative and the lack of detection of amplified products with SEQ ID NO: 43 indicating that the unknown bacteria is gram positive.

### Example 2

### Using Real-Time PCR to Determine Whether a Bacterium is Resistant to an Antibiotic

The materials and methods used in this example are now described.

The reaction mixture for real time PCR included 5 μl of H₂O, the appropriate primers and probe, and 5 μl Taqman 2× Universal Master Mix (ABI). In the reaction mixture, the final concentration of each primer was 800 nM and the final concentration of the probe was 250 nM. For these reactions, the primer set was SEQ ID NO: 44 and SEQ ID NO: 45. The probe used in the reaction mixture was SEQ ID NO: 48.

To make the reaction mixture for 10 reactions (100 μl final volume of reaction mixture), 0.8 μl of 100 μM stock solutions of each primer were added with 0.25 μl of 100 μM stock solution of minor groove binder probe and H₂O to give a final volume of 50 μl. To this solution, 50 μl of Taqman 2× Universal Master Mix was added. The final solution was vortexed, and 10 μl of the mixture was placed into one well of a 96 well plate. Before running the reaction, 1 μl of bacterial DNA (0.5 ng/μl) was added. DNA was extracted from bacteria by mixing 250 μl of bacterial culture with 250 μl of Bacterial Lysis Buffer (Roche: MagNA Pure Bacterial Lysis Buffer) and placing mixture into MagNA Lyser Green Beads tubes (Roche). Cells were disrupted using a MagNA Lyser Instrument (Roche) set at 6000 rpm for 30 seconds. After recovery, the bacterial lysate was processed using a MagNA Pure Compact instrument (Roche) and reagents supplied in the MagNA Pure Compact Nucleic Acid Isolation Kit I (Roche).

The 96 well plate containing the reaction mix were placed into an ABI 790 HT real time PCR machine. A standard real time PCR program was used, subjecting the samples to the following thermal cycle steps:

1. 50° C. for 2 minutes

2. 95° C. for 10 minutes

3. 95° C. for 15 seconds

4. 60° C. for 1 minute

Steps 3 and 4 were repeated to give a total of 35 cycles. Collected data was analyzed using software supplied with the machine.

The probe was labeled with a 5′ fluorophor (6-FAM) having a 3′ quencher (black hole) built into the end of the probe.

The results of this example are now described.

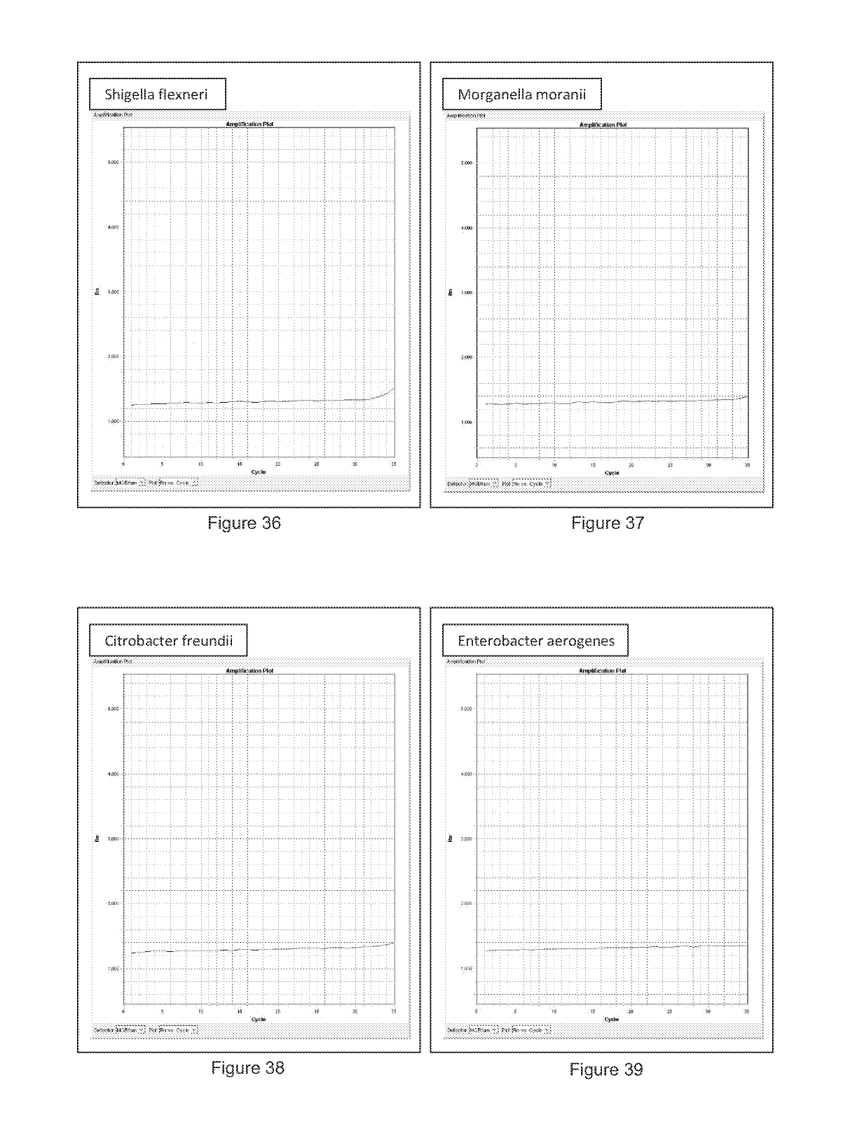

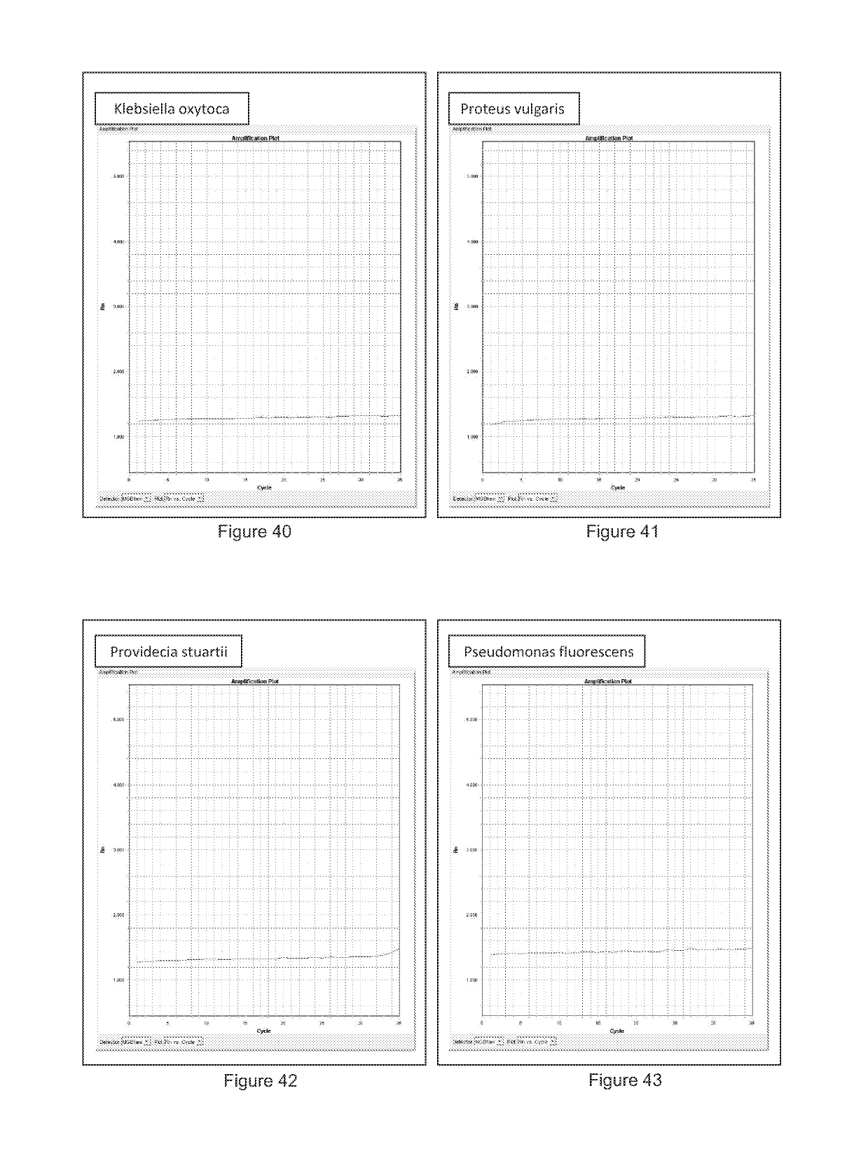

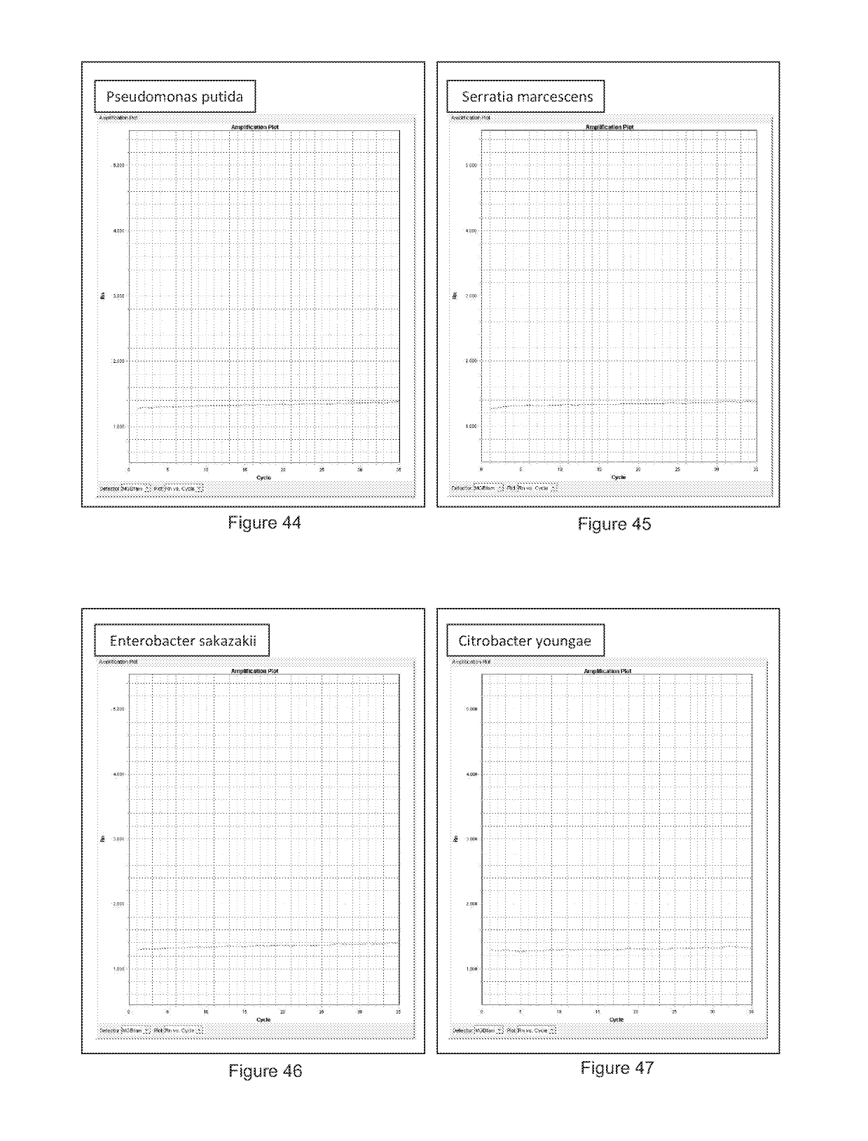

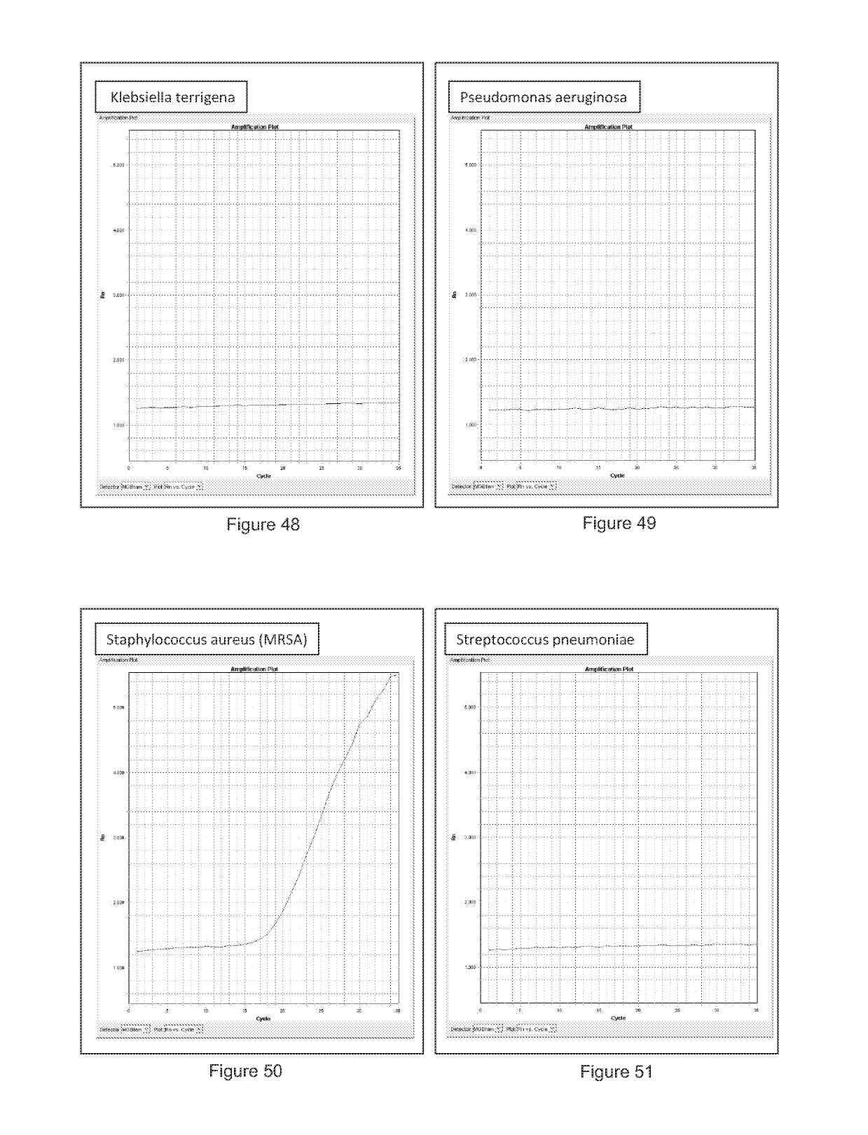

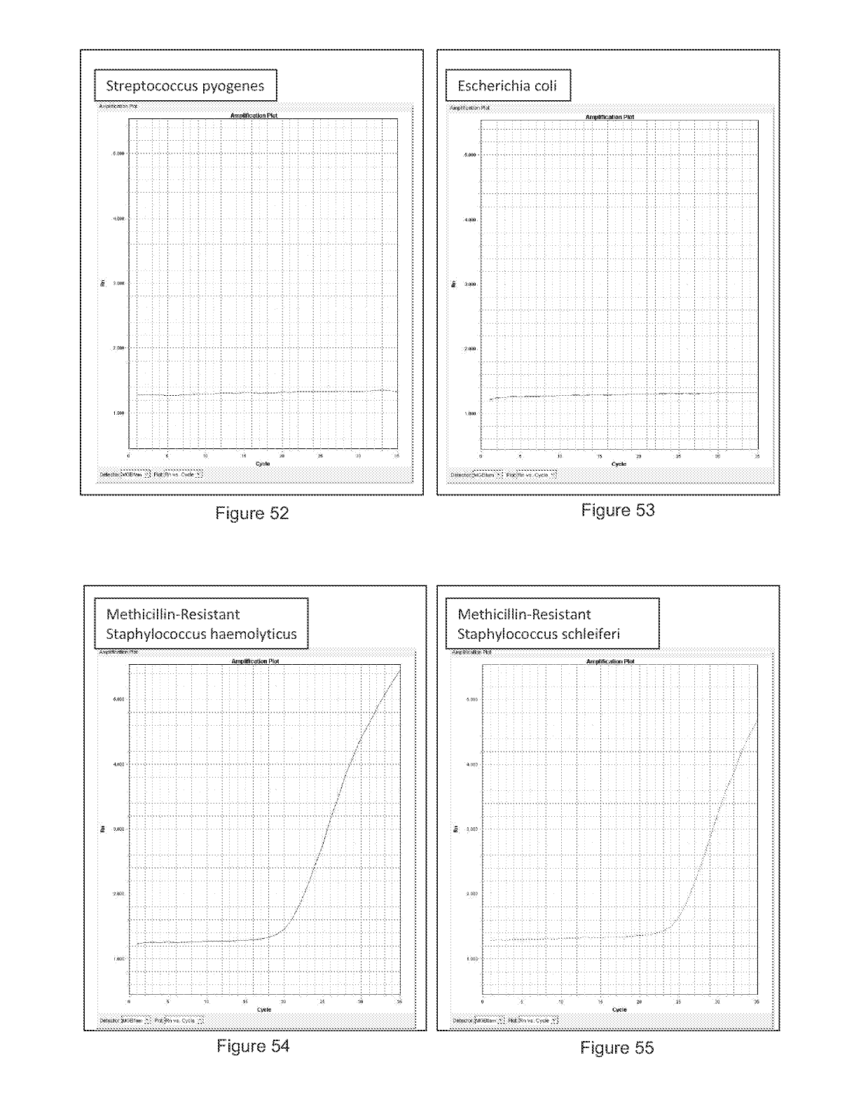

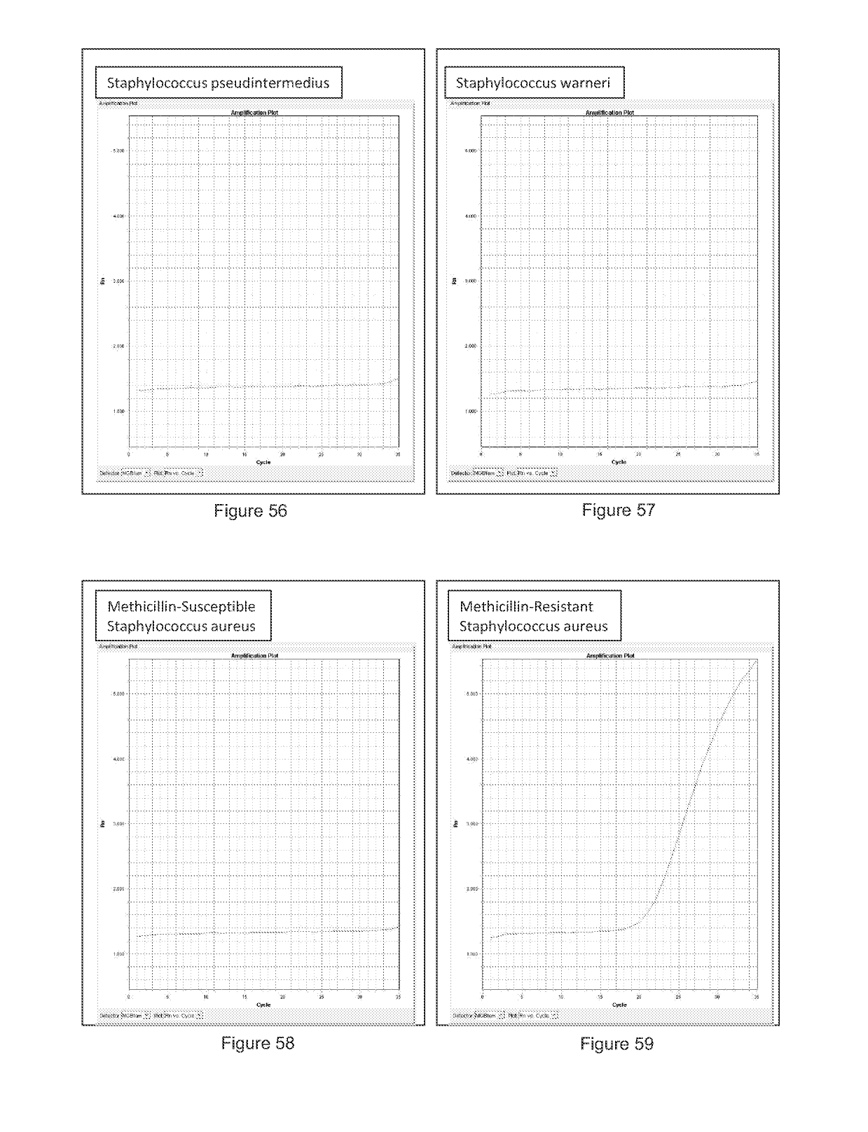

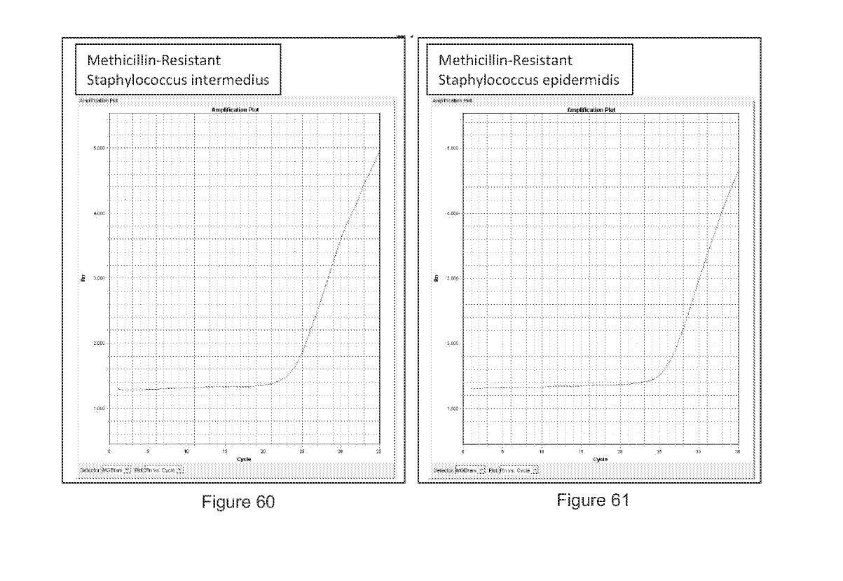

Bacterial DNA from various different species was subjected to real time PCR using SEQ ID NO: 44 and SEQ ID NO: 45 as the primer pair and SEQ ID NO: 48 as the probe. Detection of amplification products was observed when the probe in the reaction mixture was SEQ ID NO: 48 only with DNA samples isolated from bacteria known to be methicillin resistant. Conversely, amplification products were not observed when the probe in the reaction mixture was SEQ ID NO: 48 in all cases of DNA samples isolated from bacteria known not to be methicillin resistant (FIGS. 36-61).

This data shows SEQ ID NO: 44 and SEQ ID NO: 45 can be used as primers to produce amplification products from nucleic acid isolated from a large variety of bacteria, all of which can be probed with SEQ ID NO: 48 to determine whether an unknown bacteria is resistant to methicillin.

### Example 3

### Using Real-Time PCR to Determine Whether a Bacterium is Staphylococcus aureus

The materials and methods used in this example are now described.

The reaction mixture for real time PCR included 5 μl of H₂O, the appropriate primers and probe, and 5 μl Taqman 2× Universal Master Mix (ABI). In the reaction mixture, the final concentration of each primer was 800 nM and the final concentration of the probe was 250 nM. For these reactions, the primer set was SEQ ID NO: 51 and SEQ ID NO: 52. The probe used in the reaction mixture was SEQ ID NO: 53.

To make the reaction mixture for 10 reactions (100 μl final volume of reaction mixture), 0.8 μl of 100 μM stock solutions of each primer were added with 0.25 μl of 100 μM stock solution of minor groove binder probe and H₂O to give a final volume of 50 μl. To this solution, 50 μl of Taqman 2× Universal Master Mix was added. The final solution was vortexed, and 10 μl of the mixture was placed into one well of a 96 well plate. Before running the reaction, 1 μl of bacterial DNA (0.5 ng/μl) was added. DNA was extracted from bacteria by mixing 250 μl of bacterial culture with 250 μl of Bacterial Lysis Buffer (Roche: MagNA Pure Bacterial Lysis Buffer) and placing mixture into MagNA Lyser Green Beads tubes (Roche). Cells were disrupted using a MagNA Lyser Instrument (Roche) set at 6000 rpm for 30 seconds. After recovery, the bacterial lysate was processed using a MagNA Pure Compact instrument (Roche) and reagents supplied in the MagNA Pure Compact Nucleic Acid Isolation Kit I (Roche).

The 96 well plate containing the reaction mix were placed into an ABI 790 HT real time PCR machine. A standard real time PCR program was used, subjecting the samples to the following thermal cycle steps:

1. 50° C. for 2 minutes

2. 95° C. for 10 minutes

3. 95° C. for 15 seconds

4. 60° C. for 1 minute

Steps 3 and 4 were repeated to give a total of 35 cycles. Collected data was analyzed using software supplied with the machine.

The probe was labeled with a 5′ fluorophor (6-FAM) having a 3′ quencher (black hole) built into the end of the probe.

The results of this example are now described.

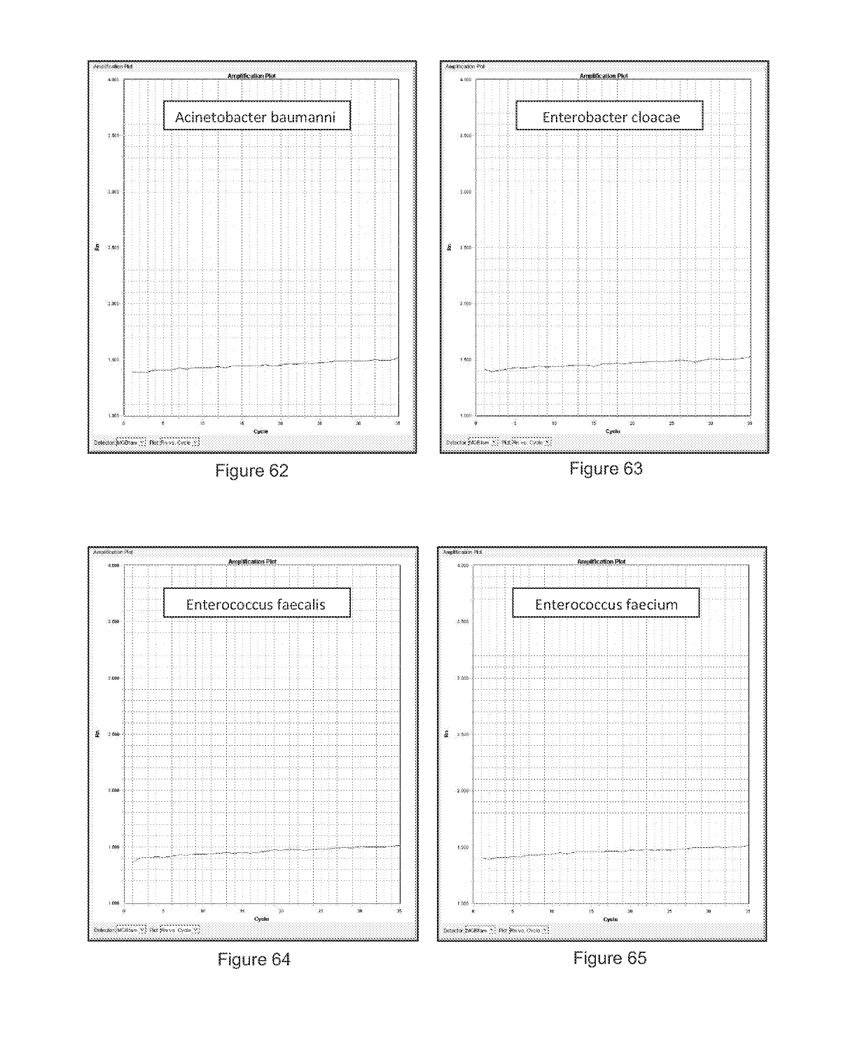

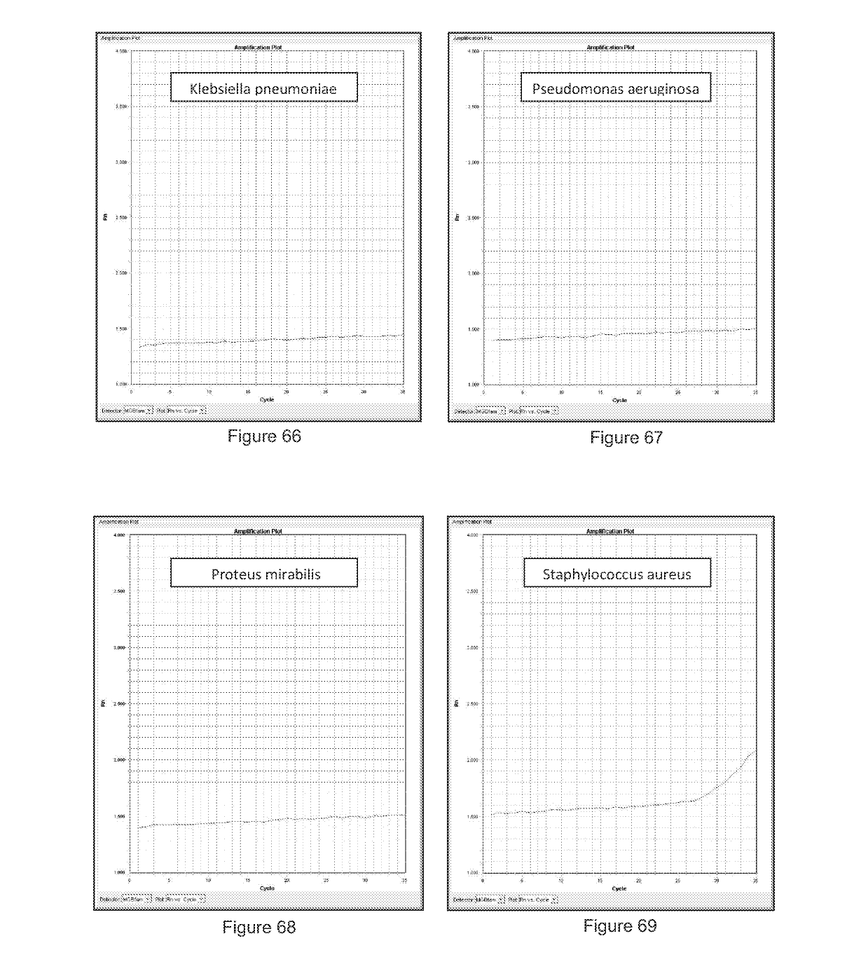

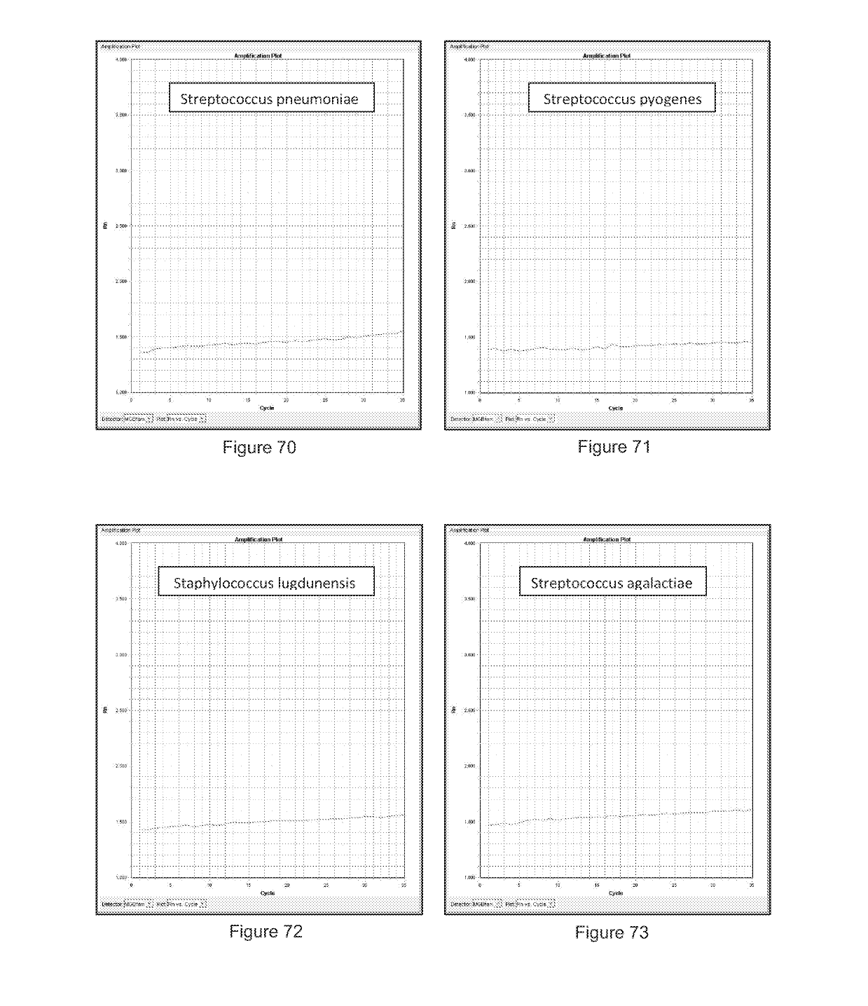

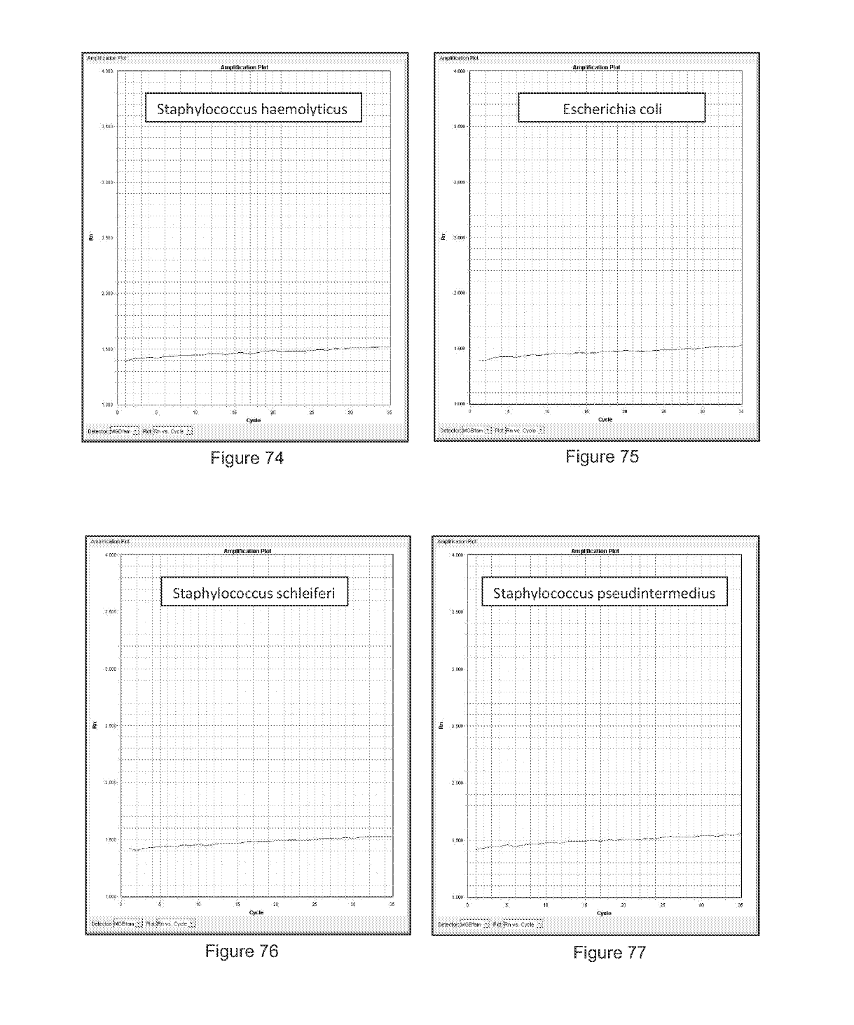

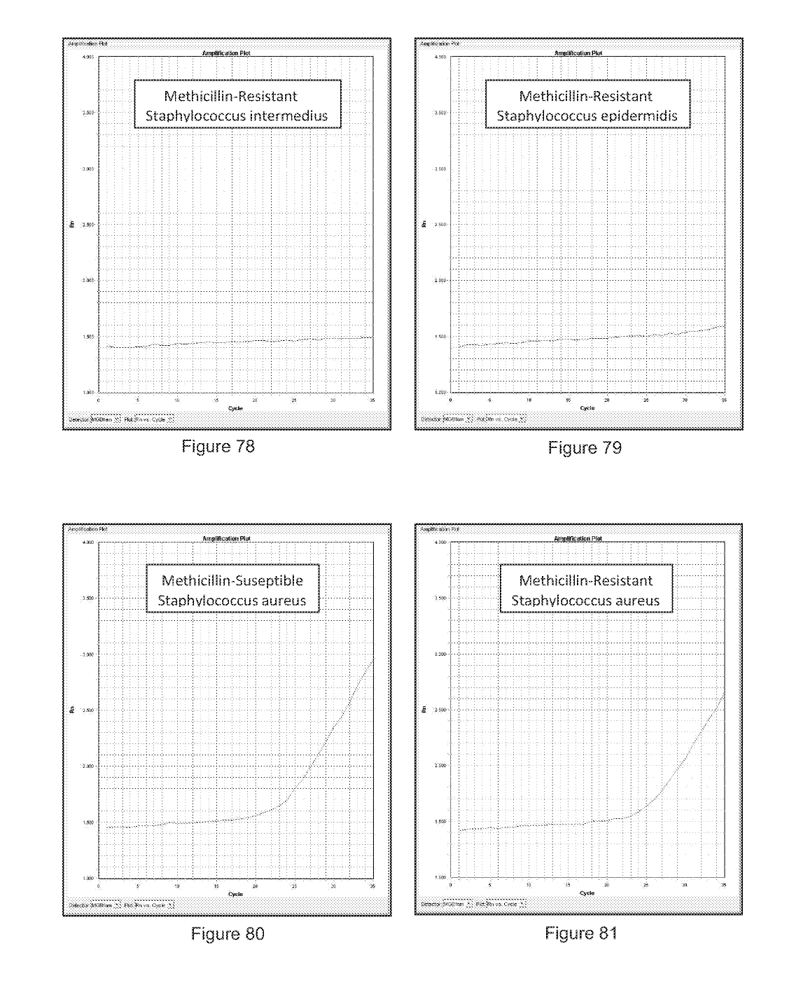

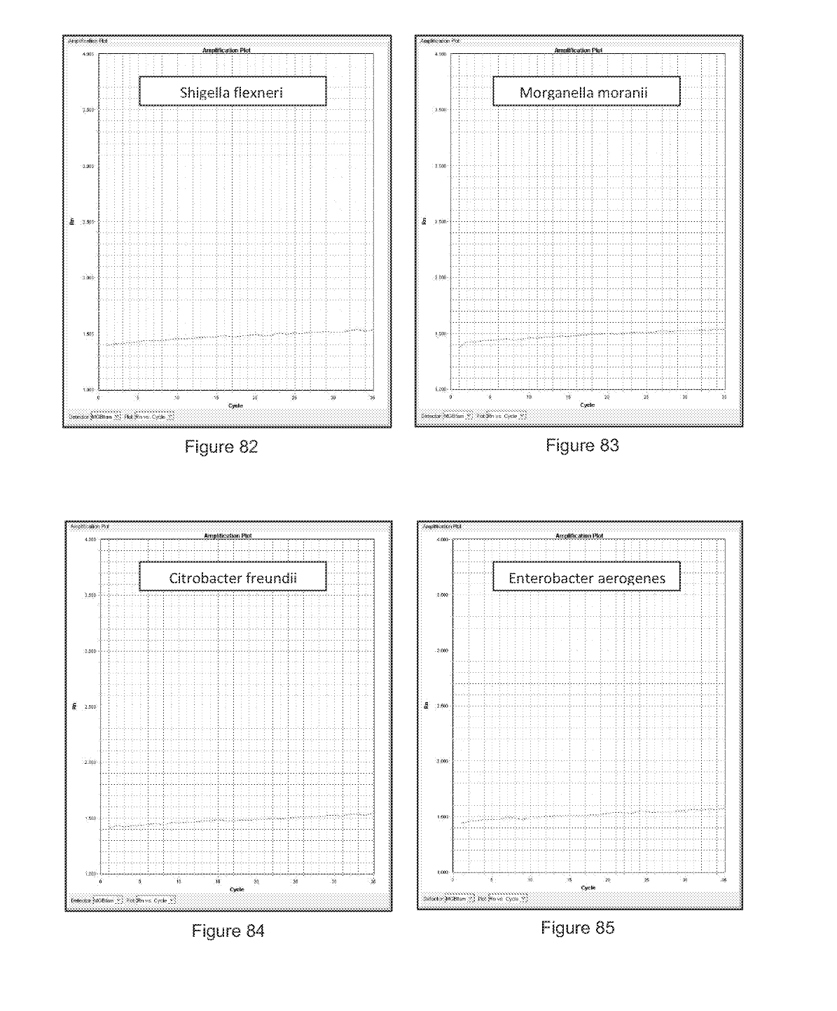

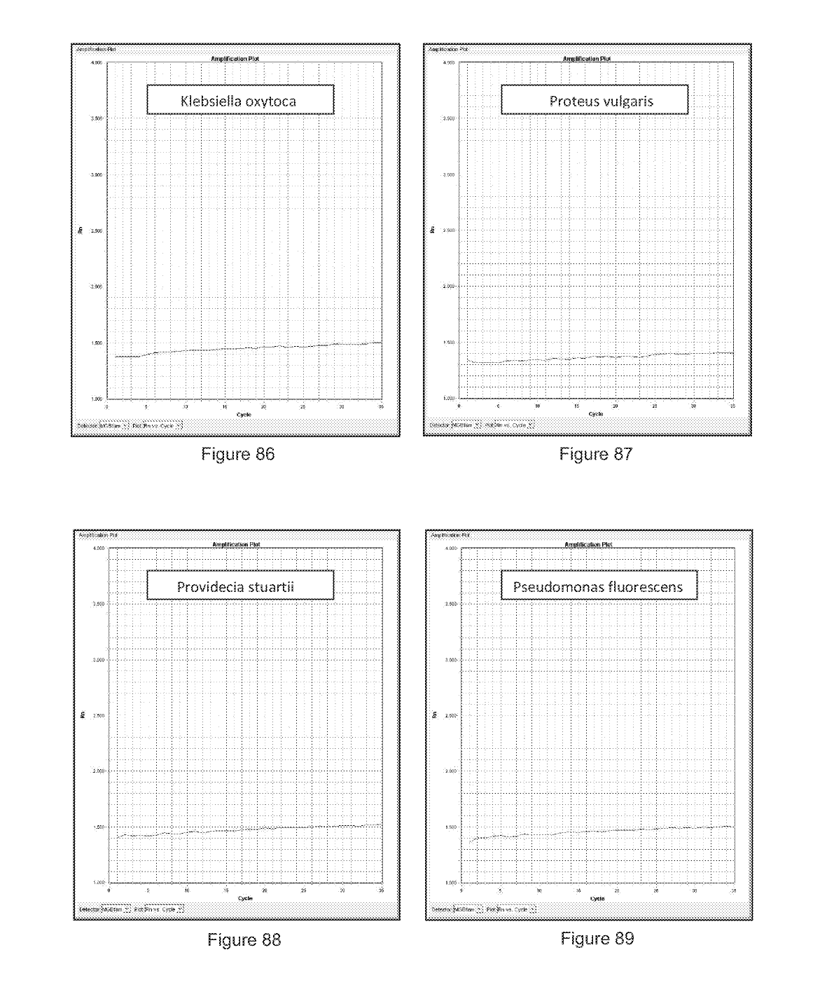

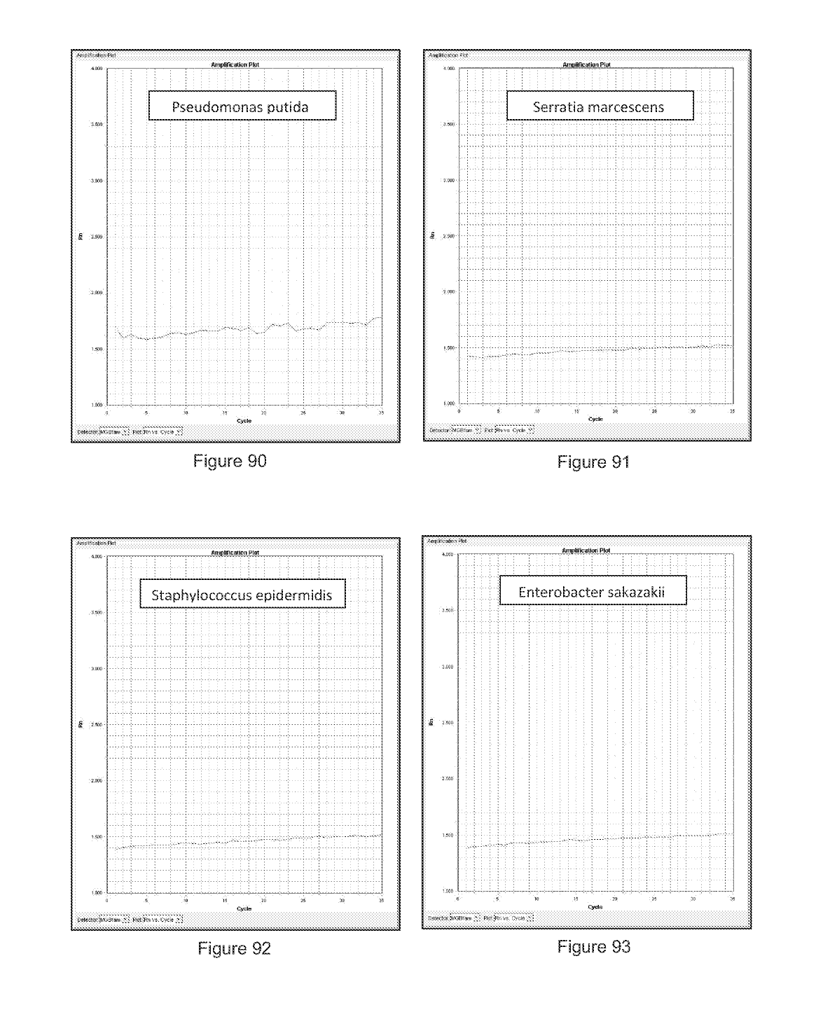

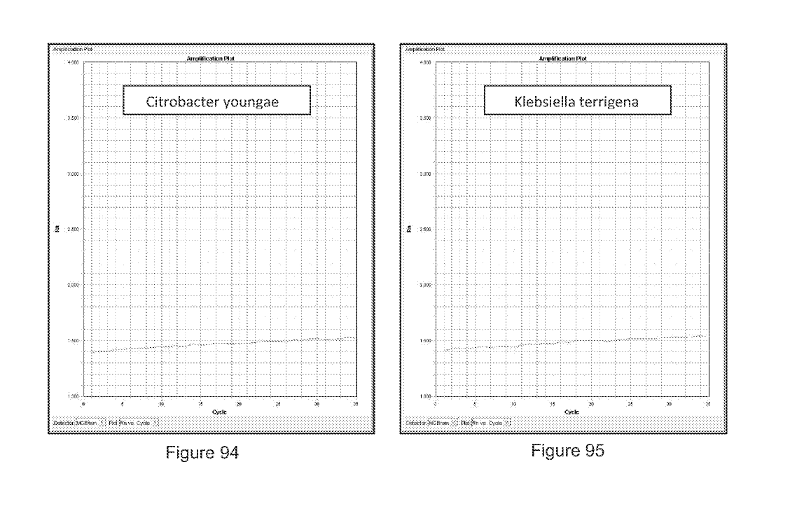

Bacterial DNA from various different species was subjected to real time PCR using SEQ ID NO: 51 and SEQ ID NO: 52 as the primer pair and SEQ ID NO: 53 as the probe. Detection of amplification products was observed when the probe in the reaction mixture was SEQ ID NO: 53 only with DNA samples isolated from bacteria known to be Staphylococcus aureus. Conversely, amplification products were not observed when the probe in the reaction mixture was SEQ ID NO: 53 in all cases of DNA samples isolated from bacteria known not to be Staphylococcus (FIGS. 62-95).

This data shows SEQ ID NO: 51 and SEQ ID NO 52 can be used as primers to produce amplification products from nucleic acid isolated from a large variety of bacteria, all of which can be probed with SEQ ID NO: 53 to determine whether an unknown bacteria is Staphylococcus aureus, where the detection of amplification products with SEQ ID NO: 53 indicates that the unknown bacteria is Staphylococcus aureus and the lack of detection of amplification products with SEQ ID NO: 53 signifies that the unknown bacteria is not Staphylococcus aureus.

### Example 4

### Using Real-Time PCR to Determine Whether a Bacterium is Staphylococcus epidermidis

The materials and methods used in this example are now described.

The reaction mixture for real time PCR included 5 μl of H₂O, the appropriate primers and probe, and 5 μl Taqman 2× Universal Master Mix (ABI). In the reaction mixture, the final concentration of each primer was 800 nM and the final concentration of the probe was 250 nM. For these reactions, the primer set was SEQ ID NO: 54 and SEQ ID NO: 55. The probe used in the reaction mixture was SEQ ID NO: 56.

To make the reaction mixture for 10 reactions (100 μl final volume of reaction mixture), 0.8 μl of 100 μM stock solutions of each primer were added with 0.25 μl of 100 μM stock solution of minor groove binder probe and H₂O to give a final volume of 50 μl. To this solution, 50 μl of Taqman 2× Universal Master Mix was added. The final solution was vortexed, and 10 μl of the mixture was placed into one well of a 96 well plate. Before running the reaction, 1 μl of bacterial DNA (0.5 ng/μl) was added. DNA was extracted from bacteria by mixing 250 μl of bacterial culture with 250 μl of Bacterial Lysis Buffer (Roche: MagNA Pure Bacterial Lysis Buffer) and placing mixture into MagNA Lyser Green Beads tubes (Roche). Cells were disrupted using a MagNA Lyser Instrument (Roche) set at 6000 rpm for 30 seconds. After recovery, the bacterial lysate was processed using a MagNA Pure Compact instrument (Roche) and reagents supplied in the MagNA Pure Compact Nucleic Acid Isolation Kit I (Roche).

The 96 well plate containing the reaction mix were placed into an ABI 790 HT real time PCR machine. A standard real time PCR program was used, subjecting the samples to the following thermal cycle steps:

1. 50° C. for 2 minutes

2. 95° C. for 10 minutes

3. 95° C. for 15 seconds

4. 60° C. for 1 minute

Steps 3 and 4 were repeated to give a total of 35 cycles. Collected data was analyzed using software supplied with the machine.

The probe was labeled with a 5′ fluorophor (6-FAM) having a 3′ quencher (black hole) built into the end of the probe.

The results of this example are now described.

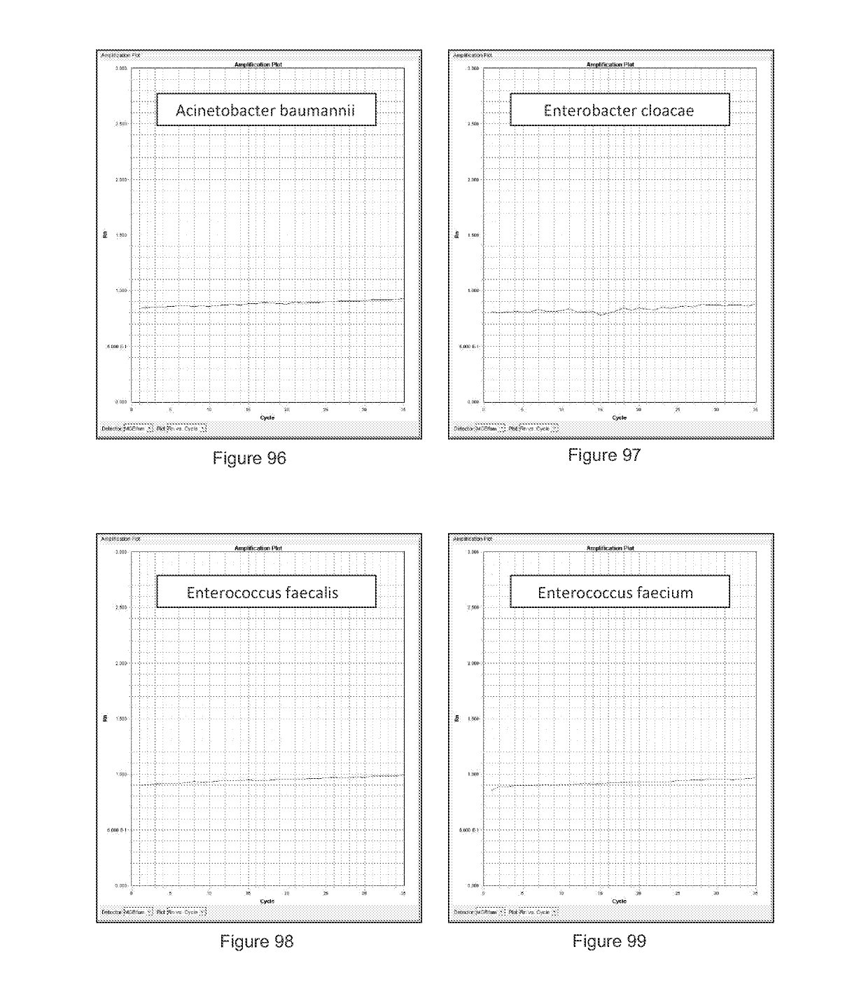

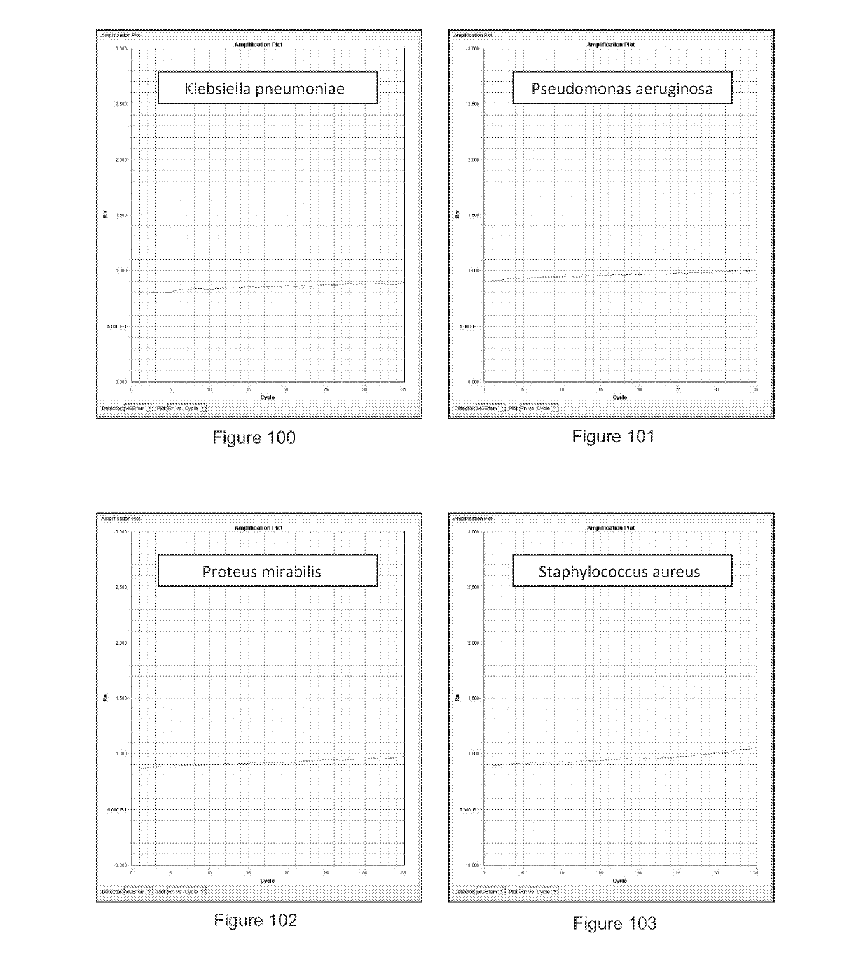

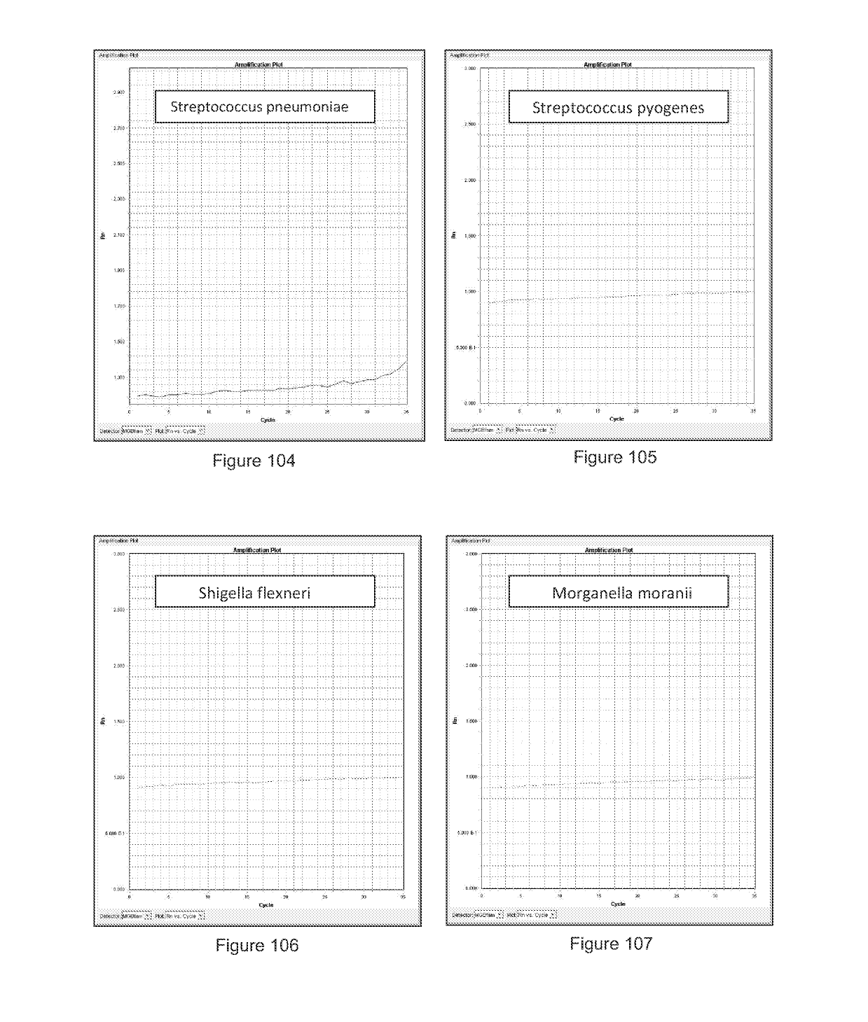

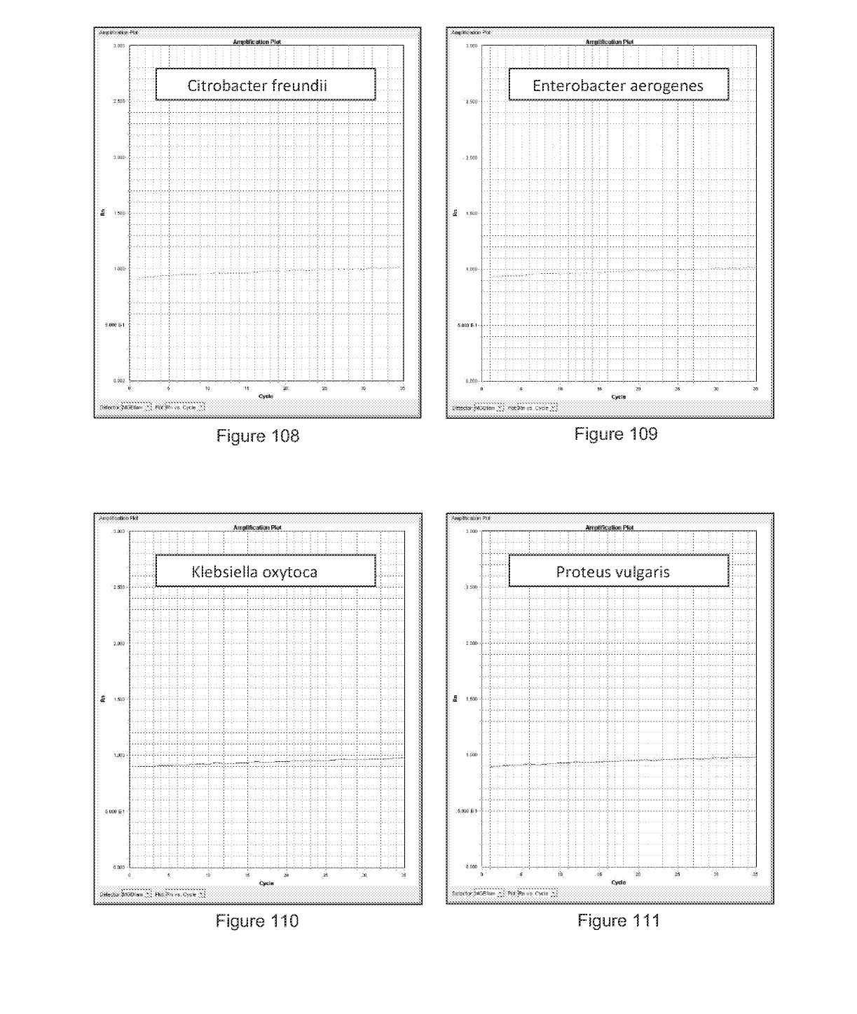

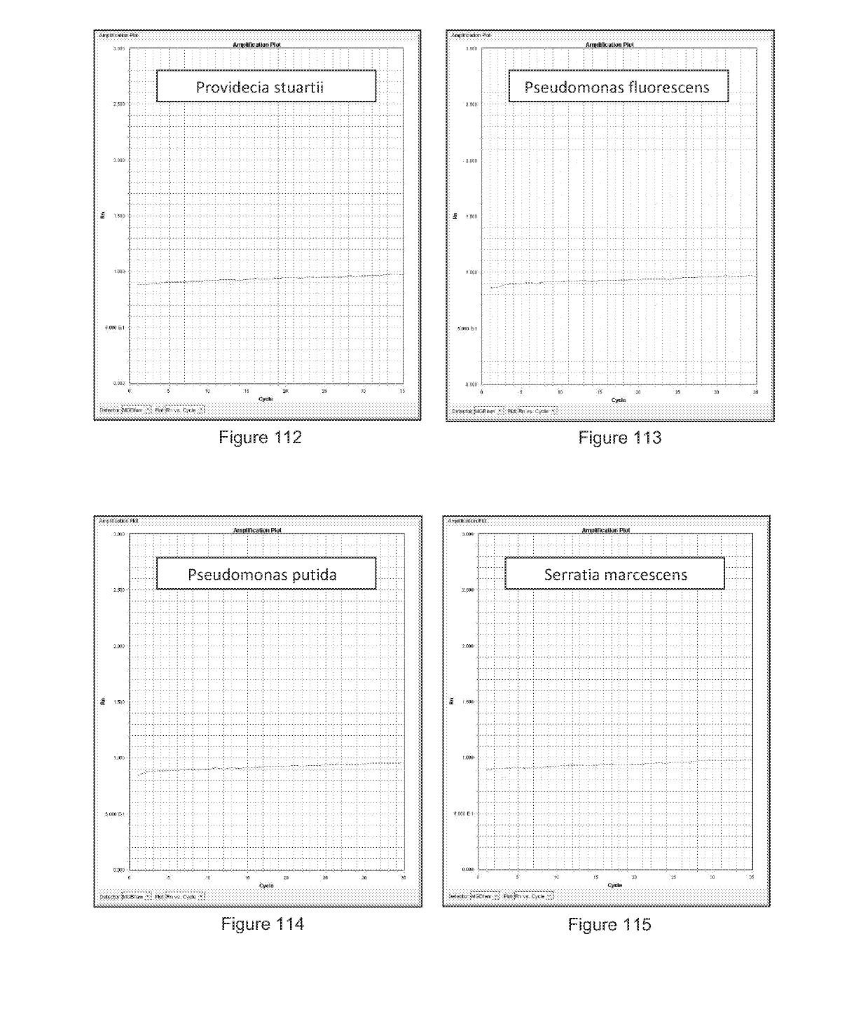

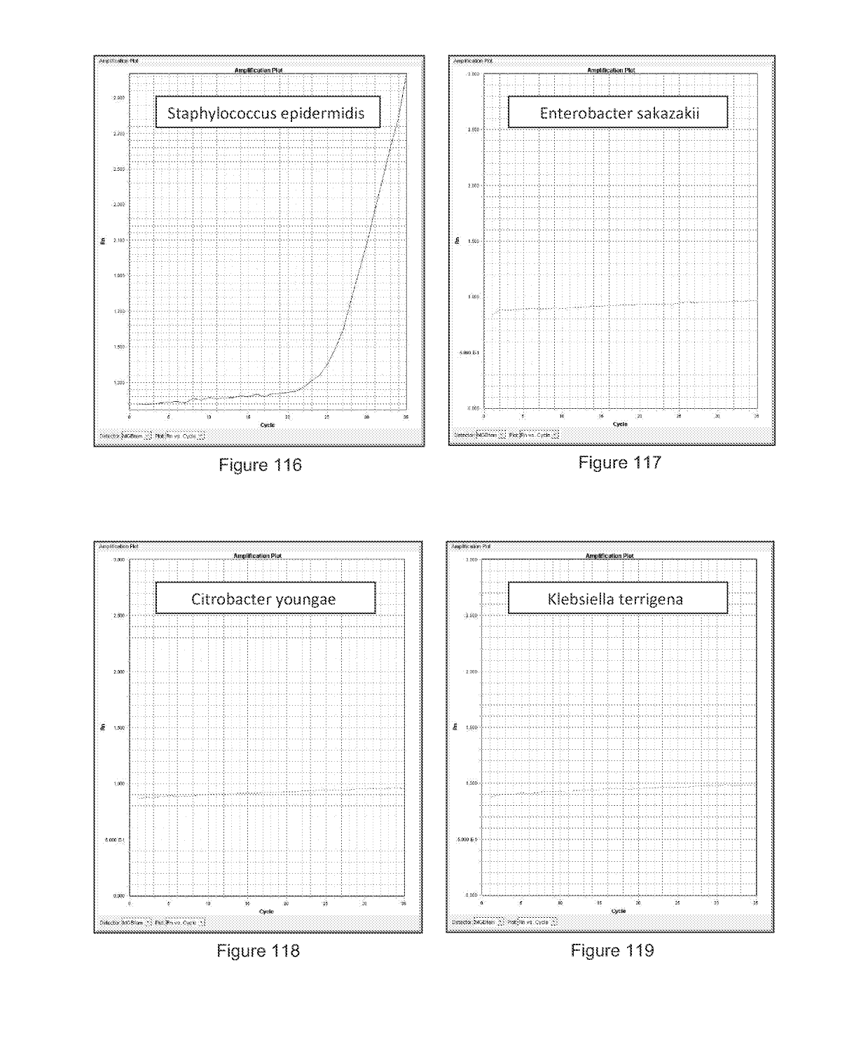

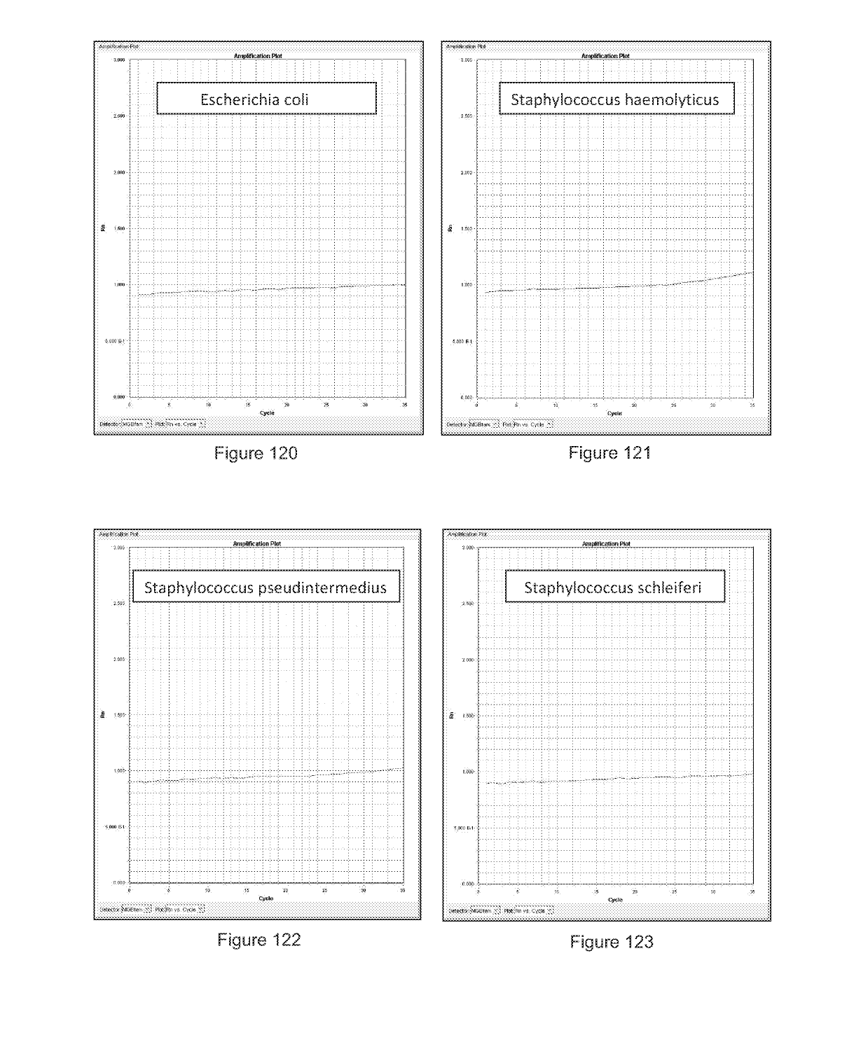

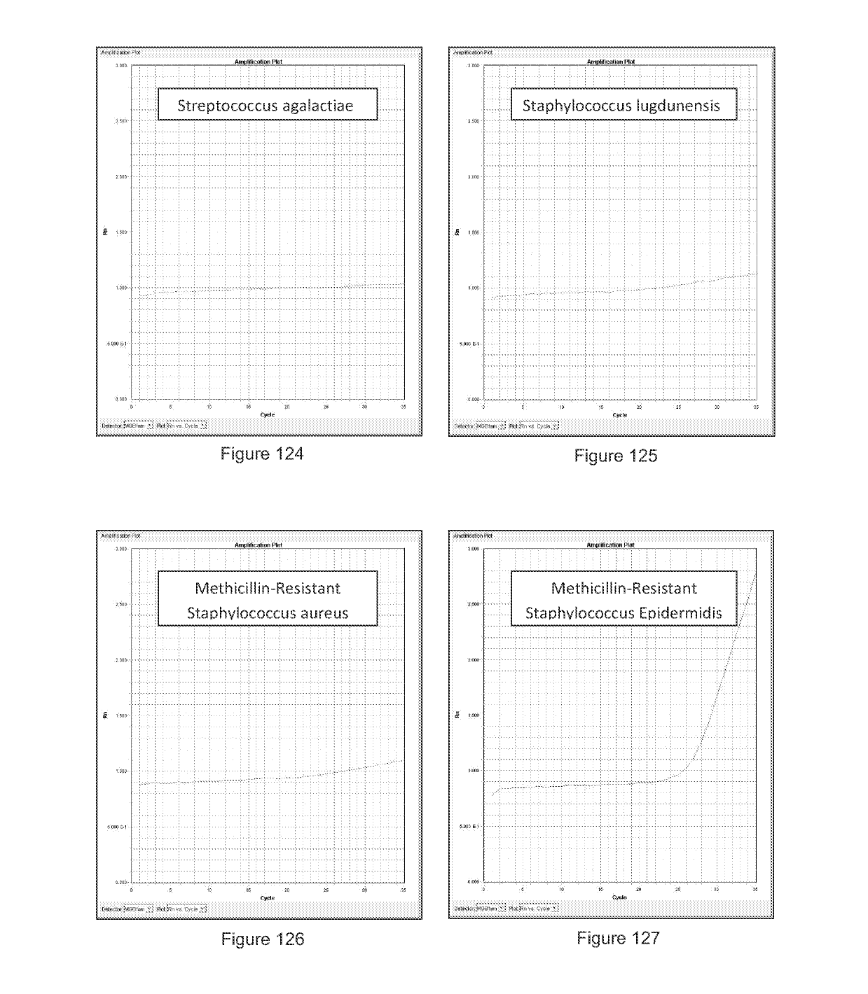

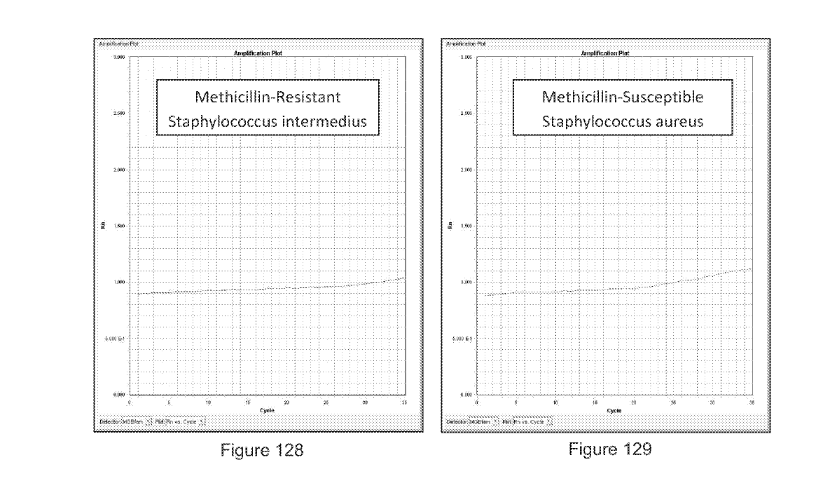

Bacterial DNA from various different species was subjected to real time PCR using SEQ ID NO: 54 and SEQ ID NO: 55 as the primer pair and SEQ ID NO: 56 as the probe. Detection of amplification products was observed when the probe in the reaction mixture was SEQ ID NO: 56 only with DNA samples isolated from bacteria known to be Staphylococcus epidermidis. Conversely, amplification products were not observed when the probe in the reaction mixture was SEQ ID NO: 56 in all cases of DNA samples isolated from bacteria known not to be Staphylococcus (FIGS. 96-129).

This data shows SEQ ID NO: 54 and SEQ ID NO: 55 can be used as primers to produce amplification products from nucleic acid isolated from a large variety of bacteria, all of which can be probed with SEQ ID NO: 56 to determine whether an unknown bacteria is Staphylococcus epidermidis, where the detection of amplification products with SEQ ID NO: 56 indicates that the unknown bacteria is Staphylococcus epidermidis and the lack of detection of amplification products with SEQ ID NO: 56 signifies that the unknown bacteria is not Staphylococcus epidermidis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of determining the Gram stain status of a bacterium present in a biological sample, the method comprising the steps of:
(a) generating a nucleic acid amplification product of a nucleic acid obtained from a bacterium in a biological sample using a pair of PCR primers, wherein the pair of PCR primers comprises TACGGGAGGCAGCAGT (SEQ ID NO: 40) and AGCAGCCGCGGTAATA (SEQ ID NO: 41); and
(b) detecting at least one of the group consisting of:
(i) the presence or absence of an amplification product that hybridizes to an oligonucleotide probe comprising the sequence: GAGCAACGCCGCGTGA (SEQ ID NO: 42), wherein the presence of the amplification product indicates that the bacteria is a Gram positive; and
(ii) the presence or absence of an amplification product that hybridizes to an oligonucleotide probe comprising the sequence: CAGCCATGCCGCGTGT (SEQ ID NO: 43), wherein the presence of the amplification product indicates that the bacteria is Gram negative.

2. The method of claim 1, wherein the detecting step comprises detecting both:
(a) the presence or absence of an amplification product that hybridizes to an oligonucleotide probe comprising the sequence: GAGCAACGCCGCGTGA (SEQ ID NO: 42), wherein the presence of the amplification product indicates that the bacteria is a Gram positive and the absence of the amplification product indicates that the bacteria is Gram negative; and
(b) the presence or absence of an amplification product that hybridizes to an oligonucleotide probe comprising the sequence: CAGCCATGCCGCGTGT (SEQ ID NO: 43), wherein the presence of the amplification product indicates that the bacteria is Gram negative and the absence of the amplification product indicates that the bacteria is Gram positive.

3. The method of claim 1, wherein the generating step and the detecting step are carried out concurrently or sequentially.

4. (canceled)

5. The method of claim 3, wherein the generating step and the detecting step are carried out concurrently in a real-time polymerase chain reaction.

6. The method of claim 1, wherein the probe selectively binds to the nucleic acid amplification product of a nucleic acid sample of a Gram positive bacteria.

7. The method of claim 1, wherein the probe selectively binds to the nucleic acid amplification product of at least one of a plurality of different Gram positive bacteria, and does not selectively bind to the nucleic acid amplification product of a nucleic acid sample from a plurality of different Gram negative bacteria under identical reactions conditions.

8. The method of claim 7, wherein the plurality of different Gram positive bacteria includes at least one species selected from the group consisting of: Bacillus species, Lactobacillus species, Micrococcus species, Mycobacterium species, Sporosarcina species, Staphylococcus species, Streptococcus species, Listeria species, Clostridium species, Corynebacterium species, and Enterococcus species.

9. The method of claim 7, wherein the plurality of different Gram positive bacteria includes at least one of the group consisting of: Enterococcus faecalis, Enterococcus faecium, Staphylococcus aureus, Staphylococcus aureus (MRSA), Streptococcus pneumoniae, Streptococcus pyogenes, Enterococcus faecalis, Staphylococcus epidermidis, Staphylococcus coagulase, and Streptococcus agalactiae.

10. The method of claim 1, wherein the probe selectively binds to the nucleic acid amplification product of a nucleic acid sample of a Gram negative bacteria.

11. The method of claim 1, wherein the probe selectively binds to the nucleic acid amplification product of at least one of a plurality of different Gram negative bacteria, and does not selectively bind to the nucleic acid amplification product of a nucleic acid sample from a plurality of different Gram positive bacteria under identical reactions conditions.

12. The method of claim 11, wherein the plurality of different Gram negative bacteria include at least one species selected from the group consisting of: Acinetobacter species, Alcaligenes species, Cytophaga species, Enterobacter species, Escherichia species, Liebsiella species, Morganella species, Proteus species, Pseudomonas species, Rhodospirillum species, Salmonella species, Serratia species, Shigella species, Citrobacter species, and Klebsiella species.

13. The method of claim 11, wherein the plurality of different Gram negative bacteria includes at least one of the group consisting of: Pseudomonas aeruginosa, Escherichia coli, Acinetobacter baumannii, Enterobacter cloacae, Klebsiella pneumoniae, Proteus mirabilis, Staphylococcus aureus, methicillin resistant Staphylococcus aureus (MRSA), Streptococcus pneumoniae, Streptococcus pyogenes, Enterococcus faecalis, Proteus mirabilis, Klebsiella pneumoniae, Citrobacter koseri, Shigella flexneri, Morganella moranii, Citrobacter freundii, Enterobacter aerogenes, Klebsiella oxytoca, Proteus vulgaris, Providecia stuartii, Pseudomonas flourescens, Pseudomonas putida, Serratia marcescens, Enterobacter sakazakii, Citrobacter youngae, and Klebsiella terrigena.

14. A method of determining the methicillin resistance status of a bacterium present in a biological sample, the method comprising the steps of:
(a) generating a nucleic acid amplification product of a nucleic acid obtained from a bacterium in a biological sample using at least one pair of PCR primers, and
(b) detecting the presence or absence of an amplification product that hybridizes to at least one oligonucleotide probe, wherein the presence of the amplification product identifies the bacterium as methicillin resistant.

15. The method of claim 14, wherein the generating step and the detecting step are carried out concurrently or sequentially.

16. (canceled)

17. The method of claim 15, wherein the generating step and the detecting step are carried out concurrently in a real-time polymerase chain reaction.

18. The method of claim 14, wherein the at least one pair of PCR primers comprises GATGGTATGTGGAAGTTAGATTG (SEQ ID NO: 44) and GACCGAAACAATGTGGAATTGG (SEQ ID NO: 45), and the at least one oligonucleotide probe comprises CATAGCGTCATTATTCCAG (SEQ ID NO: 48) or CATAGCGTCATTATTCCAGGAATGCAGAA (SEQ ID NO: 49).

19. (canceled)

20. The method of claim 14, wherein the at least one pair of PCR primers comprises GATGGCTATCGTGTCACAATCG (SEQ ID NO: 46) and TTCATATGACGTCTATCCAT (SEQ ID NO: 47), and the at least one oligonucleotide probe comprises GATTATGGCTCAGGTACTGCTATC (SEQ ID NO: 50).

21. The method of claim 14, wherein the at least one pair of PCR primers comprises GATAAGCATTGGAAATTAGATTG (SEQ ID NO: 80) and AGCTAATTCTATATTGTTTCGGTC (SEQ ID NO: 81), and the at least one oligonucleotide probe comprises CCAGACGTAATAGTACCTG (SEQ ID NO: 82).

22. A method of identifying a bacterium present in a biological sample, the method comprising the steps of:
(a) generating a nucleic acid amplification product of a nucleic acid obtained from a bacterium in a biological sample using at least one pair of PCR primers; and
(b) detecting the presence or absence of an amplification product that hybridizes to at least one oligonucleotide probe, wherein binding of the at least one oligonucleotide probe to the amplification product identifies the bacterium present in the sample.

23. The method of claim 22, wherein the generating step and the detecting step are carried out concurrently or sequentially.

24. (canceled)

25. The method of claim 23, wherein the generating step and the detecting step are carried out concurrently in a real-time polymerase chain reaction.

26. The method of claim 22, wherein the at least one pair of PCR primers comprises primers selected from the group consisting of:
(a) AGAGTTTGATCHTGGCTCAG (SEQ ID NO: 83) and CCYACTGCTGCCTCCCGTA (SEQ ID NO: 84);
(b) GGGAHGAMGWCAARTCATCAT (SEQ ID NO: 85) and CGATTACTAGCGATTCCRRCTTC (SEQ ID NO: 86); and
(c) GAAGYYGGAATCGCTAGTAATCG (SEQ ID NO: 87) and TACRGHTACCTTGTTACGACT (SEQ ID NO: 88).

27. (canceled)

28. (canceled)

29. The method of claim 22, wherein the at least one oligonucleotide probe is at least one selected from the group consisting of GAAAACAATGGCGCA (SEQ ID NO: 89), ACGGTTACCACGGAG (SEQ ID NO: 90), CTAGCCTAACTGCAAAGA (SEQ ID NO: 91), GATGACCGCCACACT (SEQ ID NO: 92), GCCTCTTGCCATCA (SEQ ID NO: 93), GCCTCATGCCATCA (SEQ ID NO: 94), GGCAGATACAAAGAG (SEQ ID NO: 95), GGCGTATACAAAGGG (SEQ ID NO: 96), GATCTGCCTGATGGC (SEQ ID NO: 97), AACGAGTCGCTAGAC (SEQ ID NO: 98), AACGAGTTGCGAAGT (SEQ ID NO: 99), ACCGTAAGGAGCCAG (SEQ ID NO: 100), CCTATTAGGAGCCAG (SEQ ID NO: 101), TTCTCTGATGTTAGC (SEQ ID NO: 102), TCCTCTGACGTTAGC (SEQ ID NO: 103), CAACGTTTCCAAAGGA (SEQ ID NO: 104), TAGCACAGAGGAGCTT (SEQ ID NO: 105), TAGCACAGAGGAGGCTT (SEQ ID NO: 106), CAGCTTGCTGCTTCGCT (SEQ ID NO: 107), TTGTAGGTGAGGTAAC (SEQ ID NO: 108), TAGCACAAGGGAGCTTG (SEQ ID NO: 109), GGACCCGCGCCGTATT (SEQ ID NO: 110), ATCTCTTAGGAGCAAAGC (SEQ ID NO: 111), and CTGAGGTTTGGTGTTTA (SEQ ID NO: 112).

30. A method of determining whether a bacterium present in a biological sample is Staphylococcus aureus, the method comprising the steps of:
(a) generating a nucleic acid amplification product of a nucleic acid obtained from a bacterium in a biological sample using a pair of PCR primers, wherein the pair of PCR primers comprises AGAGTTTGATCHTGGCTCAG (SEQ ID NO: 51) and CCTACTGCTGCCTCCCTGTA (SEQ ID NO: 52); and
(b) detecting the presence or absence of an amplification product that hybridizes to an oligonucleotide probe comprising the sequence: TTCTCTGATGTTAGC (SEQ ID NO: 53), the presence of the amplification product indicating the bacteria is Staphylococcus aureus.

31. The method of claim 30, wherein the generating step and the detecting step are carried out concurrently or sequentially.

32. (canceled)

33. The method of claim 31, wherein the generating step and the detecting step are carried out concurrently in a real-time polymerase chain reaction.

34. A method of determining whether a bacterium present in a biological sample is Staphylococcus epidermidis, the method comprising the steps of:
(a) generating a nucleic acid amplification product of a nucleic acid obtained from a bacterium in a biological sample using a pair of PCR primers, wherein the pair of PCR primers comprises AGAGTTTGATCHTGGCTCAG (SEQ ID NO: 54) and CCTACTGCTGCCTCCCTGTA (SEQ ID NO: 55); and
(b) detecting the presence or absence of an amplification product that hybridizes to an oligonucleotide probe comprising the sequence: TCCTCTGACGTTAGC (SEQ ID NO: 56), the presence of the amplification product indicating the bacteria is Staphylococcus epidermidis.

35. The method of claim 34, wherein the generating step and the detecting step are carried out concurrently or sequentially.

36. (canceled)

37. The method of claim 35, wherein the generating step and the detecting step are carried out concurrently in a real-time polymerase chain reaction.
